SEQUENCE LISTING (95 sequences)

SEQ ID NO: 1 - protein, 6 aa, synthetic construct
  REGION 1..6: note = HCDR1 for Dalotuzumab
GGYLWN

SEQ ID NO: 2 - protein, 16 aa, synthetic construct
  REGION 1..16: note = HCDR2 for Dalotuzumab
YISYDGTNNYKPSLKD

SEQ ID NO: 3 - protein, 8 aa, synthetic construct
  REGION 1..8: note = HCDR3 for Dalotuzumab
YGRVFFDY

SEQ ID NO: 4 - protein, 16 aa, synthetic construct
  REGION 1..16: note = LCDR1 for Dalotuzumab
RSSQSIVHSNGNTYLQ

SEQ ID NO: 5 - protein, 7 aa, synthetic construct
  REGION 1..7: note = LCDR2 for Dalotuzumab
KVSNRLY

SEQ ID NO: 6 - protein, 9 aa, synthetic construct
  REGION 1..9: note = LCDR3 for Dalotuzumab
FQGSHVPWT

SEQ ID NO: 7 - protein, 447 aa, synthetic construct
  REGION 1..447: note = Heavy Chain of Dalotuzumab
QVQLQESGPGLVKPSETLSLTCTVSGYSITGGYLWNWIRQPPGKGLEWIGYISYDGTNNYKPSLKDRVTISRDTSKNQFSLKLSSVTAADTAVYYCARYGRVFFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 8 - protein, 219 aa, synthetic construct
  REGION 1..219: note = Light Chain of Dalotuzumab
DIVMTQSPLSLPVTPGEPASISCRSSQSIVHSNGNTYLQWYLQKPGQSPQLLIYKVSNRLYGVPDRFSGSGSGTDFTLKISRVEAEDVGVYYCFQGSHVPWTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 9 - protein, 6 aa, synthetic construct
  REGION 1..6: note = HCDR1 of Ganitumab
SSNWWS

SEQ ID NO: 10 - protein, 16 aa, synthetic construct
  REGION 1..16: note = HCDR2 of Ganitumab
EIYHSGSTNYNPSLKS

SEQ ID NO: 11 - protein, 10 aa, synthetic construct
  REGION 1..10: note = HCDR3 of Ganitumab
WTGRTDAFDI

SEQ ID NO: 12 - protein, 19 aa, synthetic construct
  REGION 1..19: note = LCDR1 of Ganitumab
ISCRSSQSLLHSNGYNYLD

SEQ ID NO: 13 - protein, 7 aa, synthetic construct
  REGION 1..7: note = LCDR2 for Ganitumab
LGSNRAS

SEQ ID NO: 14 - protein, 9 aa, synthetic construct
  REGION 1..9: note = LCDR3 of Ganitumab
MQGTHWPLT

SEQ ID NO: 15 - protein, 448 aa, synthetic construct
  REGION 1..448: note = Heavy Chain of Ganitumab
QVQLQESGPGLVKPSGTLSLTCAVSGGSISSSNWWSWVRQPPGKGLEWIGEIYHSGSTNYNPSLKSRVTSVDKSKNQFSLKLSSVTAADTAVYYCARWTGRTDAFDIWGQGTMVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 16 - protein, 219 aa, synthetic construct
  REGION 1..219: note = Light Chain of Ganitumab
DVVMTQSPLSLPVTPGEPASISCRSSQSLLHSNGYNYLDWYLQKPGQSPQLLIYLGSNRASGVPDRFSGSGSGTDFTLKISRVEAEDVGVYYCMQGTHWPLTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 17 - protein, 5 aa, synthetic construct
  REGION 1..5: note = HCDR1 of Xentuzumab
SYWMS

SEQ ID NO: 18 - protein, 15 aa, synthetic construct
  REGION 1..15: note = HCDR2 of Xentuzumab
SITSYGSFTYADSVK

SEQ ID NO: 19 - protein, 8 aa, synthetic construct
  REGION 1..8: note = HCDR3 of Xentuzumab
NMYTHFDS

SEQ ID NO: 20 - protein, 13 aa, synthetic construct
  REGION 1..13: note = LCDR1 of Xentuzumab
SGSSSNIGSNSVS

SEQ ID NO: 21 - protein, 7 aa, synthetic construct
  REGION 1..7: note = LCDR2 of Xentuzumab
DNSKRPS

SEQ ID NO: 22 - protein, 11 aa, synthetic construct
  REGION 1..11: note = LCDR3 of Xentuzumab
QSRDTYGYYWV

SEQ ID NO: 23 - protein, 400 aa, synthetic construct
  REGION 1..400: note = Heavy Chain of Xentuzumab
QVELVESGGGLVQPGGSLRLSCAASGFTFTSYWMSWVRQAPGKGLELVSSITSYGSFTYYADSVKGRFTISRDNSKNTLYLQMNSLRAEDTAVYYCARNMYTHFDSWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDS

SEQ ID NO: 24 - protein, 216 aa, synthetic construct
  REGION 1..216: note = Light Chain of Xentuzumab
DIVLTQPPSVSGAPGQRVTISCSGSSSNIGSNSVSWYQQLPGTAPKLLIYDNSKRPSGVPDRFSGSKSGTSASLAITGLQSEDEADYYCQSRDTYGYYWVFGGGTKLTVLGQPKAAPSVTLFPPSSEELQANKATLVCLISDFYPGAVTVAWKGDSSPVKAGVETTTPSKQSNNKYAASSYLSLTPEQWKSHRSYSCQVTHEGSTVEKTVAPTECS

SEQ ID NO: 25 - protein, 5 aa, synthetic construct
  REGION 1..5: note = HCDR1 of AVE1642
SYWMH

SEQ ID NO: 26 - protein, 17 aa, synthetic construct
  REGION 1..17: note = HCDR2 of AVE1642
EINPSNGRTNYNEKFKR

SEQ ID NO: 27 - protein, 15 aa, synthetic construct
  REGION 1..15: note = HCDR3 of AVE1642
GRPDYYGSSKWYFDV

SEQ ID NO: 28 - protein, 16 aa, synthetic construct
  REGION 1..16: note = LCDR1 of AVE1642
RSSQSIVHSNVNTYLE

SEQ ID NO: 29 - protein, 7 aa, synthetic construct
  REGION 1..7: note = LCDR2 of AVE1642
KVSNRFS

SEQ ID NO: 30 - protein, 9 aa, synthetic construct
  REGION 1..9: note = LCDR3 of AVE1642
FQGSHVPPT

SEQ ID NO: 31 - protein, 124 aa, synthetic construct
  REGION 1..124: note = Heavy Chain ofAVE1642
QVQLQQSGAELVKPGASVKLSCKASGYTFTSYWMHWVKQRPGQGLEWIGEINPSNGRTNYNEKFKRKATLTVDKSSSTAYMQLSSLTSEDSAVYYFARGRPDYYGSSKWYFDVWGAGTTVTVSS

SEQ ID NO: 32 - protein, 113 aa, synthetic construct
  REGION 1..113: note = Light Chain of AVE1642
DVLMTQTPLSLPVSLGDQASISCRSSQSIVHSNVNTYLEWYLQKPGQSPKLLIYKVSNRFSGVPDRFSGSGSGTDFTLRISRVEAEDLGIYYCFQGSHVPPTFGGGTKLEIKR

SEQ ID NO: 33 - protein, 10 aa, synthetic construct
  REGION 1..10: note = HCDR1 of Figitumumab
GFTFSSYAMN

SEQ ID NO: 34 - protein, 17 aa, synthetic construct
  REGION 1..17: note = HCDR2 of Figitumumab
AISGSGGTTFYADSVKG

SEQ ID NO: 35 - protein, 16 aa, synthetic construct
  REGION 1..16: note = HCDR3 of Figitumumab
DLGWSDSYYYYYGMDV

SEQ ID NO: 36 - protein, 11 aa, synthetic construct
  REGION 1..11: note = LCDR1 of Figitumumab
RASQGIRNDLG

SEQ ID NO: 37 - protein, 7 aa, synthetic construct
  REGION 1..7: note = LCDR2 of Figitumumab
AASRLHR

SEQ ID NO: 38 - protein, 9 aa, synthetic construct
  REGION 1..9: note = LCDR3 of Figitumumab
LQHNSYPCS

SEQ ID NO: 39 - protein, 451 aa, synthetic construct
  REGION 1..451: note = Heavy Chain of Figitumumab
EVQLLESGGGLVQPGGSLRLSCTASGFTFSSYAMNWVRQAPGKGLEWVSAISGSGGTTFYADSVKGRFTISRDNSRTTLYLQMNSLRAEDTAVYYCAKDLGWSDSYYYYYGMDVWGQGTTVTVSSASTKGPSVFPLAPCSRSTSESTAALGCLVKDYFPEPVTVSWNSGALTSGVHTFPAVLQSSGLYSLSSVVTVPSSNFGTQTYTCNVDHKPSNTKVDKTVERKCCVECPPCPAPPVAGPSVFLFPPKPKDTLMISRTPEVTCVVVDVSHEDPEVQFNWYVDGVEVHNAKTKPREEQFNSTFRVVSVLTVVHQDWLNGKEYKCKVSNKGLPAPIEKTISKTKGQPREPQVYTLPPSREEMTKNQVSLTCLVKGFYPSDIAVEWESNGQPENNYKTTPPMLDSDGSFFLYSKLTVDKSRWQQGNVFSCSVMHEALHNHYTQKSLSLSPGK

SEQ ID NO: 40 - protein, 214 aa, synthetic construct
  REGION 1..214: note = Light Chain of Figitumumab
DIQMTQFPSSLSASVGDRVTITCRASQGIRNDLGWYQQKPGKAPKRLIYAASRLHRGVPSRFSGSGSGTEFTLTISSLQPEDFATYYCLQHNSYPCSFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 41 - protein, 5 aa, synthetic construct
  REGION 1..5: note = HCDR1 of Dusigitumab
SYDIN

SEQ ID NO: 42 - protein, 17 aa, synthetic construct
  REGION 1..17: note = HCDR2 of Dusigitumab
WMNPNSGNTGYAQKFQG

SEQ ID NO: 43 - protein, 11 aa, synthetic construct
  REGION 1..11: note = HCDR3 of Dusigitumab
DPYYYYYGMDV

SEQ ID NO: 44 - protein, 13 aa, synthetic construct
  REGION 1..13: note = LCDR1 of Dusigitumab
SGSSSNIENNHVS

SEQ ID NO: 45 - protein, 7 aa, synthetic construct
  REGION 1..7: note = LCDR2 of Dusigitumab
DNNKRPS

SEQ ID NO: 46 - protein, 12 aa, synthetic construct
  REGION 1..12: note = LCDR3 of Dusigitumab
ETWDTSLSAGRV

SEQ ID NO: 47 - protein, 446 aa, synthetic construct
  REGION 1..446: note = Heavy Chain of Dusigitumab
QVQLVQSGAEVKKPGASVKVSCKASGYTFTSYDINWVRQATGQGLEWMGWMNPNSGNTGYAQKFQGRVTMTRNTSISTAYMELSSLRSEDTAVYYCARDPYYYYYGMDVWGQGTTVTVSSASTKGPSVFPLAPCSRSTSESTAALGCLVKDYFPEPVTVSWNSGALTSGVHTFPAVLQSSGLYSLSSVVTVPSSNFGTQTYTCNVDHKPSNTKVDKTVERKCCVECPPCPAPPVAGPSVFLFPPKPKDTLMISRTPEVTCVVVDVSHEDPEVQFNWYVDGVEVHNAKTKPREEQFNSTFRVVSVLTVVHQDWLNGKEYKCKVSNKGLPAPIEKTISKTKGQPREPQVYTLPPSREEMTKNQVSLTCLVKGFYPSDIAVEWESNGQPENNYKTTPPMLDSDGSFFLYSKLTVDKSRWQQGNVFSCSVMHEALHNHYTQKSLSLSPGK

SEQ ID NO: 48 - protein, 217 aa, synthetic construct
  REGION 1..217: note = Light Chain of Dusigitumab
QSVLTQPPSVSAAPGQKVTISCSGSSSNIENNHVSWYQQLPGTAPKLLIYDNNKRPSGIPDRFSGSKSGTSATLGITGLQTGDEADYYCETWDTSLSAGRVFGGGTKLTVLGQPKAAPSVTLFPPSSEELQANKATLVCLISDFYPGAVTVAWKADSSPVKAGVETTTPSKQSNNKYAASSYLSLTPEQWKSHRSYSCQVTHEGSTVEKTVAPTECS

SEQ ID NO: 49 - protein, 5 aa, synthetic construct
  REGION 1..5: note = HCDR1 of Cixutumumab
SYAIS

SEQ ID NO: 50 - protein, 16 aa, synthetic construct
  REGION 1..16: note = HCDR2 of Cixutumumab
GIIPIFGTANYAQKFQ

SEQ ID NO: 51 - protein, 21 aa, synthetic construct
  REGION 1..21: note = HCDR3 of Cixutumumab
APLRFLEWSTQDHYYYYYMDV

SEQ ID NO: 52 - protein, 11 aa, synthetic construct
  REGION 1..11: note = LCDR1 of Cixutumumab
QGDSLRSYYAT

SEQ ID NO: 53 - protein, 7 aa, synthetic construct
  REGION 1..7: note = LCDR2 of Cixutumumab
GENKRPS

SEQ ID NO: 54 - protein, 11 aa, synthetic construct
  REGION 1..11: note = LCDR3 of Cixutumumab
KSRDGSGQHLV

SEQ ID NO: 55 - protein, 460 aa, synthetic construct
  REGION 1..460: note = Heavy Chain of Cixutumumab
EVQLVQSGAEVKKPGSSVKVSCKASGGTFSSYAISWVRQAPGQGLEWMGGIIPIFGTANYAQKFQGRVTITADKSTSTAYMELSSLRSEDTAVYYCARAPLRFLEWSTQDHYYYYYMDVWGKGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 56 - protein, 214 aa, synthetic construct
  REGION 1..214: note = Light Chain of Cixutumumab
SSELTQDPAVSVALGQTVRITCQGDSLRSYYATWYQQKPGQAPILVIYGENKRPSGIPDRFSGSSSGNTASLTITGAQAEDEADYYCKSRDGSGQHLVFGGGTKLTVLGQPKAAPSVTLFPPSSEELQANKATLVCLISDFYPGAVTVAWKADSSPVKAGVETTTPSKQSNNKYAASSYLSLTPEQWKSHRSYSCQVTHEGSTVEKTVAPAECS

SEQ ID NO: 57 - protein, 5 aa, synthetic construct
  REGION 1..5: note = HCDR1 of BIIB022
IYRMQ

SEQ ID NO: 58 - protein, 17 aa, synthetic construct
  REGION 1..17: note = HCDR2 of BIIB022
GISPSGGTTWYADSVKG

SEQ ID NO: 59 - protein, 11 aa, synthetic construct
  REGION 1..11: note = HCDR3 of BIIB022
WSGGSGYAFDI

SEQ ID NO: 60 - protein, 10 aa, synthetic construct
  REGION 1..10: note = LCDR1 of BIIB022
QASRDIRNYN

SEQ ID NO: 61 - protein, 7 aa, synthetic construct
  REGION 1..7: note = LCDR2 of BIIB022
DASSLQT

SEQ ID NO: 62 - protein, 9 aa, synthetic construct
  REGION 1..9: note = LCDR3 of BIIB022
QQFDSLPHT

SEQ ID NO: 63 - protein, 120 aa, synthetic construct
  REGION 1..120: note = Heavy Chain of BIIB022
EVQLLESGGGLVQPGGSLRLSCAASGFTFSIYRMQWVRQAPGKGLEWVSGISPSGGTTWYADSVKGRFTISRDNSKNTLYLQMNSLRAEDTAVYYCARWSGGSGYAFDIWGQGTMVTVSS

SEQ ID NO: 64 - protein, 107 aa, synthetic construct
  REGION 1..107: note = Light Chain of BIIB022
DIQMTQSPLSLSASVGDRVTITCQASRDIRNYLNWYQQKPGKAPKLLIYDASSLQTGVPSRFGGSGSGTDFSFTIGSLQPEDIATYYCQQFDSLPHTFGQGTKLEIK

SEQ ID NO: 65 - protein, 448 aa, synthetic construct
  REGION 1..448: note = HCDR1 of Robatumumab
EVQLVQSGGGLVKPGGSLRLSCAASGFTFSSFAMHWVRQAPGKGLEWISVIDTRGATYYADSVKGRFTISRDNAKNSLYLQMNSLRAEDTAVYYCARLGNFYYGMDVWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 66 - protein, 214 aa, synthetic construct
  REGION 1..214: note = HCDR2 of Robatumumab
EIVLTQSPGTLSVSPGERATLSCRASQSIGSSLHWYQQKPGQAPRLLIKYASQSLSGIPDRFSGSGSGTDFTLTISRLEPEDFAVYYCHQSSRLPHTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 67 - protein, 10 aa, synthetic construct
  REGION 1..10: note = HCDR1of Istiratumab
GFMFSRYPMH

SEQ ID NO: 68 - protein, 16 aa, synthetic construct
  REGION 1..16: note = HCDR2 of Istiratumab
ISGSGGATPYADSVKG

SEQ ID NO: 69 - protein, 13 aa, synthetic construct
  REGION 1..13: note = HCDR3 of Istiratumab
DFYQILTGNAFDY

SEQ ID NO: 70 - protein, 11 aa, synthetic construct
  REGION 1..11: note = LCDR1 of Istiratumab
RASQGISSYLA

SEQ ID NO: 71 - protein, 7 aa, synthetic construct
  REGION 1..7: note = LCDR2 of Istiratumab
AKSTLQS

SEQ ID NO: 72 - protein, 9 aa, synthetic construct
  REGION 1..9: note = LCDR3 of Istiratumab
QQYWTFPLT

SEQ ID NO: 73 - protein, 720 aa, synthetic construct
  REGION 1..720: note = Heavy Chain of Istiratumab
EVQLLQSGGGLVQPGGSLRLSCAASGFMFSRYPMHWVRQAPGKGLEWVGSISGSGGATPYADSVKGRFTISRDNSKNTLYLQMNSLRAEDTAVYYCAKDFYQILTGNAFDYWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGGGGGSGGGGSGGGGSQVQLVQSGGGLVQPGGSLRLSCAASGFTFDDYAMHWVRQAPGKGLEWVAGISWDSGSTGYADSVKGRFTISRDNAKNSLYLQMNSLRAEDTALYYCARDLGAYQWVEGFDYWGQGTLVTVSSASTGGGGSGGGGSGGGGSGGGGSSYELTQDPAVSVALGQTVRITCQGDSLRSYYASWYQQKPGQAPVLVIYGKNNRPSGIPDRFSGSTSGNSASLTITGAQAEDEADYYCNSRDSPGNQWVFGGGTKVTVLG

SEQ ID NO: 74 - protein, 214 aa, synthetic construct
  REGION 1..214: note = Light Chain of Istiratumab
DIQMTQSPSSLSASLGDRVTITCRASQGISSYLAWYQQKPGKAPKLLIYAKSTLQSGVPSRFSGSGSGTDFTLTISSLQPEDSATYYCQQYWTFPLTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 75 - protein, 10 aa, synthetic construct
  REGION 1..10: note = HCDR1 of AVE1642 (Humanized form of murine antibody EM164)
GYTFTSYWMH

SEQ ID NO: 76 - protein, 10 aa, synthetic construct
  REGION 1..10: note = HCDR2 of AVE1642 (Humanized form of murine antibody EM164)
EINPSNGRTN

SEQ ID NO: 77 - protein, 17 aa, synthetic construct
  REGION 1..17: note = HCDR2 of AVE1642 (Humanized form of murine antibody EM164)
EINPSNGRTNYNQKFQG

SEQ ID NO: 78 - protein, 124 aa, synthetic construct
  REGION 1..124: note = VH2 Domain of AVE1642
QVQLVQSGAEVVKPGASVKLSCKASGYTFTSYWMHWVKQRPGQGLEWIGEINPSNGRTNYNQKFQGKATLTVDKSSSTAYMQLSSLTSEDSAVYYFARGRPDYYGSSKWYFDVWGQGTTVTVSS

SEQ ID NO: 79 - protein, 123 aa, synthetic construct
  REGION 1..123: note = VH3 domain of AVE1642
QVQLVQSGAEVVKPGASVKLSCKASGYTFTSYWMHWVKQRPGQGLEWIGEINPSNGRTNYNQKFQGKATLTVDKSSSTAYMQLSSLTSEDSAVYYFARGRPDYYGSSKWYFDVWGQGTTVTVS

SEQ ID NO: 80 - protein, 113 aa, synthetic construct
  REGION 1..113: note = VL2 domain of AVE1642
DVVMTQTPLSLPVSLGDPASISCRSSQSIVHSNVNTYLEWYLQKPGQSPRLLIYKVSNRFSGVPDRFSGSGAGTDFTLRISRVEAEDLGIYYCFQGSHVPPTFGGGTKLEIKR

SEQ ID NO: 81 - protein, 113 aa, synthetic construct
  REGION 1..113: note = VL3 domain of AVE1642
DVLMTQTPLSLPVSLGDPASISCRSSQSIVHSNVNTYLEWYLQKPGQSPKLLIYKVSNRFSGVPDRFSGSGAGTDFTLRISRVEAEDLGIYYCFQGSHVPPTFGGGTKLEIKR

SEQ ID NO: 82 - protein, 113 aa, synthetic construct
  REGION 1..113: note = VL4 of AVE1642
DVLMTQTPLSLPVSLGDPASISCRSSQSIVHSNVNTYLEWYLQKPGQSPRLLIYKVSNRFSGVPDRFSGSGAGTDFTLRISRVEAEDLGIYYCFQGSHVPPTFGGGTKLEIKR

SEQ ID NO: 83 - protein, 113 aa, synthetic construct
  REGION 1..113: note = VL5 domain of AVE1642
DVVMTQTPLSLPVSLGDPASISCRSSQSIVHSNVNTYLEWYLQKPGQSPKLLIYKVSNRFSGVPDRFSGSGAGTDFTLRISRVEAEDLGIYYCFQGSHVPPTFGGGTKLEIKR

SEQ ID NO: 84 - protein, 5 aa, synthetic construct
  REGION 1..5: note = CDRH1 of Antibody 1 (Teprotumumab)
SYGMH

SEQ ID NO: 85 - protein, 17 aa, synthetic construct
  REGION 1..17: note = CDRH2 of Antibody 1 (Teprotumumab)
IIWFDGSSTYYADSVRG

SEQ ID NO: 86 - protein, 9 aa, synthetic construct
  REGION 1..9: note = CDRH3 of Antibody 1 (Teprotumumab)
ELGRRYFDL

SEQ ID NO: 87 - protein, 11 aa, synthetic construct
  REGION 1..11: note = CDRL1 of Antibody 1 (Teprotumumab)
RASQSVSSYLA

SEQ ID NO: 88 - protein, 7 aa, synthetic construct
  REGION 1..7: note = CDRL2 of Antibody 1 (Teprotumumab)
DASKRAT

SEQ ID NO: 89 - protein, 10 aa, synthetic construct
  REGION 1..10: note = CDRL3 of Antibody 1 (Teprotumumab)
QQRSKWPPWT

SEQ ID NO: 90 - protein, 118 aa, synthetic construct
  REGION 1..118: note = VH of Antibody 1 (Teprotumumab)
QVELVESGGGVVQPGRSQRLSCAASGFTFSSYGMHWVRQAPGKGLEWVAIIWFDGSSTYYADSVRGRFTISRDNSKNTLYLQMNSLRAEDTAVYFCARELGRRYFDLWGRGTLVSVSS

SEQ ID NO: 91 - protein, 108 aa, synthetic construct
  REGION 1..108: note = VL of Antibody 1 (Teprotumumab)
EIVLTQSPATLSLSPGERATLSCRASQSVSSYLAWYQQKPGQAPRLLIYDASKRATGIPARFSGSGSGTDFTLTISSLEPEDFAVYYCQQRSKWPPWTFGQGTKVESK

SEQ ID NO: 92 - protein, 17 aa, synthetic construct
  REGION 1..17: note = CDRH2 of antibody 2
IIWFDGSSKYYGDSVKG

SEQ ID NO: 93 - protein, 7 aa, synthetic construct
  REGION 1..7: note = CDRL2 of Antibody 2
DASNRAT

SEQ ID NO: 94 - protein, 118 aa, synthetic construct
  REGION 1..118: note = VH of Antibody 2
QVQLVESGGGVVQPGRSLRLSCAASGFTFSSYGMHWVRQAPGKGLEWMAIIWFDGSSKYYGDSVKGRFTISRDNSKNTLYLQMNSLRADDTAVYYCARELGRRYFDLWGRGTLVTVSS

SEQ ID NO: 95 - protein, 108 aa, synthetic construct
  REGION 1..108: note = VL of Antibody 2
EIVLTQSPATLSLSPGERATLSCRASQSVSSYLAWYQQKPGQAPRLLIYDASNRATGIPARFSGSGSGTDFTLTISSLEPEDFAVYYCQQRSKWPPWTFGQGTKVEIK

(19) United States
(12) Patent Application Publication
(10) Pub. No.: US 2023/0270851 A1
(43) Pub. Date: Aug. 31, 2023

(54) METHODS FOR THE TREATMENT OF IDIOPATHIC ORBITAL INFLAMMATION AND RELATED CONDITIONS

(71) Applicant: HORIZON THERAPEUTICS IRELAND DAC, Dublin 2 (IE)

(72) Inventors: Bobby KORN, San Diego, CA (US); Jeffrey SHERMAN, Lincolnshire, IL (US)

(21) Appl. No.: 18/057,637

(22) Filed: Nov. 21, 2022

Related U.S. Application Data
(63) Continuation of application No. PCT/US2021/034506, filed on May 27, 2021.
(60) Provisional application No. 63/031,439, filed on May 28, 2020.

(51) Int. Cl. A61K 39/395; A61K 39/00; A61P 37/02

(52) U.S. Cl. CPC A61K 39/395; A61K 39/00; A61P 37/02; A61K 2039/505

(57) ABSTRACT

Provided herein are methods of treating or reducing idiopathic orbital inflammation (IOI), also known as idiopathic orbital inflammatory syndrome, nonspecific orbital inflammation (NSOI), orbital inflammatory pseudotumor, and orbital inflammatory syndrome (OIS), or one or more symptoms thereof, in a subject with IOI, comprising administering to the subject an effective amount of an insulin-like growth factor 1 receptor (IGF-1R) inhibitor, and pharmaceutical composition comprising an amount of an insulin-like growth factor 1 receptor (IGF-1R) inhibitor effective for treating or reducing one or more effects of idiopathic orbital inflammation (IOI), or one or more symptoms thereof, in a subject with IOI.

This application is a continuation of International Application No. PCT/US2021/034506, filed on May 27, 2027, which claims the benefit of priority of U.S. Provisional Application No. 63/031,439 filed May 28, 2020, the disclosure of which is hereby incorporated by reference as if written herein in its entirety.

### SEQUENCE LISTING

The instant application contains a Sequence Listing which has been submitted electronically in XML format and is hereby incorporated by reference in its entirety. Said XML copy, created on Nov. 21, 2022, is named 58651_815_301_SL.xml and is 126,649 bytes in size.

Idiopathic orbital inflammation (IOI), also known as idiopathic orbital inflammatory syndrome, nonspecific orbital inflammation (NSOI), orbital inflammatory pseudotumor, and orbital inflammatory syndrome (OIS) is the most common cause of painful orbital mass in adults. IOI can be localized or diffuse. When localized, inflammation can affect the extraocular muscles (orbital myositis), lacrimal gland (dacryoadenitis), sclera (scleritis), uvea (uveitis), and the superior orbital fissure and cavernous sinus (Tolosa-Hunt syndrome). Others include periscleritis, perineuritis, and mass. When diffuse, IOI may diffusely involve the orbital fatty tissues. Multiple IOI classification systems have previously been proposed in the literature. See, e.g., Bijlsma et al., “Evaluation of classification systems for nonspecific idiopathic orbital inflammation,” Orbit, 2012 Aug.; 31(4):238-45, evaluating past classification systems and proposing a new best practice classification system based on a combination of histopathology and localization which provides a repeatable, easy to use, plausible, and complete classification system of IOIs. Based on histopathology, IOI is classified into four categories, namely, classic, granulomatous, sclerosing, and not otherwise specified (NOS). Based on localization, IOI is classified into six categories, namely, diffuse, extraocular muscle, lacrimal gland, optic nerve, sclera and not otherwise specified (NOS).

IOI can both radiologically and clinically mimic a malignant process. Therefore, it has traditionally been a diagnosis of exclusion, only after all other causes of inflammation have been eliminated. See, e.g., Chundury R et al., “Nonspecific Orbital Inflammation (Idiopathic orbital inflammation, Orbital inflammatory syndrome, Orbital pseudotumor)” American Academy of Ophthalmology (AAO) EyeWiki, last modified Apr. 8, 2020, at 14:25, accessed May 28, 2020 at 11:09. To overcome the diagnosis by exclusion methodology for IOI, Mombaerts et al. performed a consensus-establishing exercise combining scientific literature with expert opinion of 35 clinicians by using a modified Delphi consensus process. A set of clinical and radiologic criteria for the diagnosis of JOT were developed. Twenty-three consensus diagnostic criteria of JOT (CDCIOI) proposed by Mombaerts will assist clinicians to safely diagnose JOT and avoid misdiagnosis of other orbital entities. CDCIOI described in Mombaerts et al., “Consensus on Diagnostic Criteria of Idiopathic Orbital Inflammation Using a Modified Delphi Approach,”JAMAOphthalmol. 2017; 135(7):769-776, are incorporated herein in their entirety.

Treatment options for IOI are varied and can include surgery, steroids, chemotherapeutic agents, and irradiation. No therapy has been specifically approved for IOI.

The etiology and pathogenesis of IOI is currently unknown. Both infectious and immune-mediate etiologies have been implicated. IOI is a benign noninfectious, inflammatory process of the orbit; characterized by a polymorphous lymphoid infiltrate with varying degrees of fibrosis, without a known local or systemic cause. See “Orbits, Eyelids, and Lacrimal System,” AAO, BCSC Section 7, 2011-2012 pg. 59. An immune-mediated pathophysiology is supported by increased cytokines observed in patients, and favorable, rapid responses of inflammation to systemic corticosteroids and other immunosuppressive agents.

IOI is the third most common orbital disease after thyroid eye disease and orbital lymphoma. IO has been shown to account for up to 8-10% of orbital disorders. See Yuen S J & Rubin P, “Idiopathic orbital inflammation: ocular mechanisms and clinicopathology,” Ophthalmol Clin N Am 2002; 15:121-126.

There are no drugs approved in the United States specifically for the treatment of IOI. Typically, mild cases are treated with NSID drugs, and severe cases with syst corticosteroids. When IOI is found to be resistant to or intolerant to corticosteroid therapy, radiation therapy or various surgeries may also be used. There are scattered reports of successful use of cytotoxic chemotherapeutics, calcineurin inhibitors, and biologic drugs targeting TNF-α (e.g., adalimumab, infliximab), CD20 (rituximab), and IL-6 (tocilizumab) in individual or very small numbers of patients.

There remains a need for new therapies to treat idiopathic orbital inflammation and related conditions. Additional therapies to treat the symptoms of idiopathic orbital inflammation would be useful, but a therapy which can reduce and repair the damage in idiopathic orbital inflammation would be even more beneficial for patients.

Insulin-like growth factor proteins are essential in regulating cell growth and death. There are three types of proteins involved in the IGF system, including two insulin-like growth factor ligands (IGF1 and IGF2), two insulin-like growth factor receptors (IGF-1R and IGF-2R), and 6 insulin-like growth factor binding proteins (IGFBP1-6). There are indications that IGF system plays a role in many forms of cancer and many immune-mediated and inflammatory diseases. In these diseases there is a higher level of IGF present in the tissue and/or blood of the patients. It is believed the higher level of IGF plays a role in preventing the normal apoptosis process of the cells involved. Therefore, stopping the overstimulation IGF pathway is expected to be beneficial to IOI patients.

### DETAILED DESCRIPTION

Provided herein are inhibitors of insulin-like growth factor 1 receptor (IGF-1R) for the use in the treatment of idiopathic orbital inflammation and symptoms thereof.

### EMBODIMENTS

Provided as Embodiment 1 is a method of treating or reducing one or more effects of idiopathic orbital inflammation (IOI), or one or more symptoms thereof, in a subject with IOI, comprising administering to the subject an effective amount of an insulin-like growth factor 1 receptor (IGF-1R) inhibitor.

Also provided are the following embodiments.

Embodiment 2: the method of Embodiment 1, wherein the IOI is classified histopathologically as one of classic, granulomatous, sclerosing, and not otherwise specified (NOS), and/or is classified based on localization as one or more of diffuse, extraocular muscle, lacrimal gland, optic nerve, sclera and not otherwise specified (NOS).

Embodiment 3: the method of Embodiment 2, wherein the IOI is classic IOI.

Embodiment 4: the method of Embodiment 2, wherein the IOI is granulomatous IOI.

Embodiment 5: the method of Embodiment 2, wherein the IOI is sclerosing IOI.

Embodiment 6: the method of any of Embodiments 2-5, wherein the IOI is diffuse IOI.

Embodiment 7: the method of any of Embodiments 2-5, wherein the IOI is localized to the extraocular muscle.

Embodiment 8: the method of any of Embodiments 2-5, wherein the IOI is localized to the lacrimal gland.

Embodiment 9: the method of any of Embodiments 2-5, wherein the IOI is localized to the optic nerve.

Embodiment 10: the method of any of Embodiments 2-5, wherein the IOI is localized to the sclera.

Embodiment 11: the method of any of Embodiments 2-5, wherein the IOI is NOS.

Embodiment 12: the method of any of Embodiments 1-11, wherein the one or more effects of IOI that is/are reduced is/are chosen from:
eyelid edema;
ptosis;
proptosis;
globe displacement;
impairment of eye motility;
disease progression, e.g., to involve one more of the lacrimal gland, the superior rectus muscle, the lateral rectus muscle, and the inferomedial orbit;
nongranulomatous, chronic inflammation;
sclerosing inflammation; and
nonspecific granulomatous inflammation.

Embodiment 13: The method of any of Embodiments 1-12, wherein the IGF-1R inhibitor is an antibody, or an antigen binding fragment thereof, or a small molecule or a salt or polymorph thereof.

Embodiment 14: The method of Embodiment 13 wherein said IGF-1R inhibitor is chosen from teprotumumab, ganitumab, figitumumab, MEDI-573, cixutumumab, dalotuzumab, robatumumab, AVE1642, BIIB022, xentuzumab, istiratumab, linsitinib, picropodophyllin, BMS-754807, BMS-536924, BMS-554417, GSK1838705A, GSK1904529A, NVP-AEW541, NVP-ADW742, GTx-134, AG1024, KW-2450, PL-2258, INSM-18, AZD3463, AZD9362, BI885578, BI893923, TT-100, XL-228, and A-928605.

Embodiment 15: The method of Embodiment 13 wherein said IGF-1R inhibitor is an antibody, or an antigen binding fragment thereof.

Embodiment 16: The method of Embodiment 15 wherein said IGF-1R inhibitor is a human, chimeric human, or humanized monoclonal antibody, or an antigen binding fragment thereof, suitable for human therapy.

Embodiment 17: The method of Embodiment 15, wherein the antibody or antigen binding fragment thereof is administered intradermally, subcutaneously (SC), intravenously (IV), or by inhalation.

Embodiment 18: The method of Embodiment 17 wherein the antibody or antigen binding fragment thereof is administered intravenously (IV) or subcutaneously (SC).

Embodiment 19: The method of Embodiment 18 wherein the antibody or antigen binding fragment thereof is administered IV.

Embodiment 20: The method of Embodiment 19, wherein the IGF-1R inhibitor is administered by IV infusion to the subject.

Embodiment 21: The method of any of Embodiments 15-20 wherein said antibody is chosen from teprotumumab, ganitumab, figitumumab, MEDI-573, cixutumumab, dalotuzumab, robatumumab, AVE1642, BIIB022, xentuzumab, and istiratumab, or an antigen binding fragment of any of the foregoing.

Embodiment 22: The method of any of Embodiments 1-21, wherein the IGF-1R inhibitor is an antibody, or antigen binding fragment thereof, that has a binding affinity (K_{D}) of 10⁻⁸ M or less for the IGF-1R.

Embodiment 23: The method of Embodiment 22, wherein the antibody, or antigen binding fragment thereof, has a binding affinity (K_{D}) of 10⁻¹³ to 10⁻⁹ M for the IGF-1R.

Embodiment 24: The method of any of Embodiments 1-23, wherein the antibody, or antigen binding fragment thereof, has an IC50 values for the IGF1 and IGF2 to IGF-1R of no more than 2 nM.

Embodiment 25: The method of any of Embodiments 1-24, wherein the IGF-1R inhibitor is an antibody, or an antigen binding fragment thereof, and comprises a heavy chain comprising CDR1, CDR2, and CDR3 and a light chain comprising CDR1, CDR2 and CDR3, wherein the heavy chain CDR1, CDR2, and CDR3 amino acid sequences and light chain CDR1, CDR2, and CDR3 amino acid sequences are at least 90% identical to (i) the amino acid sequences of SEQ ID NOs:83-89, respectively; or (ii) the amino acid sequences of SEQ ID NOs:84, 92, 86, 87, 93, and 89, respectively.

Embodiment 26: The method of Embodiment 25 wherein the antibody, or an antigen binding fragment thereof, comprises: (i) heavy chain CDR1, CDR2, and CDR3 amino acid sequences and light chain CDR1, CDR2, and CDR3 amino acid sequences as set forth in SEQ ID NOs: 83-89, respectively; or (ii) heavy chain CDR1, CDR2, and CDR3 amino acid sequences and light chain CDR1, CDR2, and CDR3 amino acid sequences as set forth in SEQ ID NOs: 83, 92, 86, 87, 93, and 89, respectively.

Embodiment 27: The method of any of Embodiments 1-26, wherein the IGF-1R inhibitor is an antibody, or an antigen binding fragment thereof, and comprises: (i) a heavy chain variable region having at least 80% sequence identity to the amino acid sequence of SEQ ID NO: 90 and a light chain variable region having at least 80% sequence identity to the amino acid sequence of SEQ ID NO: 91; or (ii) a heavy chain variable region having at least 80% sequence identity to the amino acid sequence of SEQ ID NO: 94 and a light chain variable region having at least 80% sequence identity to the amino acid sequence of SEQ ID NO: 95.

Embodiment 28: The method of Embodiment 27 wherein the antibody is teprotumumab, or an antigen binding fragment thereof.

Embodiment 29. The method of Embodiment 28 wherein the teprotumumab is dosed at:
a) 1-60 mg/kg or 75-4500 mg IV every 3 weeks; or
b) 0.6-40 mg/kg or 45-3000 mg IV every 2 weeks; or
c) 0.3-20 mg/kg; or 22-1500 mg IV weekly.

Embodiment 30: The method of Embodiment 28, wherein the IGF-1R inhibitor is an antibody, or antigen binding fragment thereof, and is administered at a dosage about 1 mg/kg to about 5 mg/kg antibody as a first dose.

Embodiment 31: The method Embodiment 28, wherein the IGF-1R inhibitor is an antibody, or antigen binding fragment thereof, and is administered at a dosage about 5 mg/kg to about 10 mg/kg antibody as a first dose.

Embodiment 32: The method of any of Embodiments 30-31, wherein the antibody, or antigen binding fragment thereof, is administered at a dosage about 5 mg/kg to about 20 mg/kg antibody in subsequent doses.

Embodiment 33: The method of Embodiment 32, wherein the said antibody, or antigen binding fragment thereof, is administered at a dosage about 10 mg/kg as a first dose; and about 20 mg/kg antibody in subsequent doses.

Embodiment 34: The method of any of Embodiments 30-33, wherein subsequent doses are administered every three weeks for at least 21 weeks.

Embodiment 35: The method of Embodiment 21 wherein the antibody is ganitumab, or an antigen binding fragment thereof.

Embodiment 36: The method of Embodiment 35 wherein the ganitumab is dosed at:
a) 1-60 mg/kg or 75-4500 mg IV every 3 weeks; or
b) 0.6-40 mg/kg or 45-3000 mg IV every 2 weeks; or
c) 0.3-20 mg/kg; or 22-1500 mg IV weekly.

Embodiment 37: The method of Embodiment 21 wherein the antibody is figitumumab, or an antigen binding fragment thereof.

Embodiment 38: The method of Embodiment 37 wherein the figitumumab is dosed at:
a) 1-60 mg/kg or 75-4500 mg IV every 3 weeks; or
b) 0.6-40 mg/kg or 45-3000 mg IV every 2 weeks; or
c) 0.3-20 mg/kg or 22-1500 mg IV weekly.

Embodiment 39: The method of Embodiment 21 wherein the antibody is cixutumumab, or an antigen binding fragment thereof.

Embodiment 40: The method of Embodiment 39 wherein the cixutumumab is dosed at:
a) 1-45 mg/kg or 75-3400 mg IV every 3 weeks; or
b) 0.6-30 mg/kg or 45-2300 mg IV every 2 weeks; or
c) 0.3-15 mg/kg Or 22-1200 mg IV weekly.

Embodiment 41: The method of Embodiment 21 wherein the antibody is dalotuzumab, or an antigen binding fragment thereof.

Embodiment 42: The method of Embodiment 41 wherein the dalotuzumab is dosed at:
a) 1-90 mg/kg or 75-6800 mg IV every 3 weeks; or
b) 0.6-60 mg/kg or 45-4500 mg IV every 2 weeks; or
c) 0.3-30 mg/kg or 22-2300 mg IV weekly.

Embodiment 43: The method of Embodiment 21 wherein the antibody is robatumumab, or an antigen binding fragment thereof.

Embodiment 44: The method of Embodiment 43 wherein the robatumumab is dosed at:
a) 1-75 mg/kg or 75-5700 mg IV every 3 weeks; or
b) 0.6-50 mg/kg or 45-3800 mg IV every 2 weeks; or
c) 0.3-25 mg/kg or 22-1900 mg IV weekly.

Embodiment 45: The method of Embodiment 21 wherein the antibody is xentuzumab, or an antigen binding fragment thereof.

Embodiment 46: The method of Embodiment 45 wherein the xentuzumab is dosed at:
a) 1-112 mg/kg or 75-8400 mg IV every 3 weeks; or
b) 0.6-75 mg/kg or 45-5700 mg IV every 2 weeks; or
c) 0.3-38 mg/kg or 22-2900 mg IV weekly.

Embodiment 47: The method of Embodiment 21 wherein the antibody is istiratumab, or an antigen binding fragment thereof.

Embodiment 48: The method of Embodiment 47 wherein the istiratumab is dosed at:
a) 1-112 mg/kg or 75-8400 mg IV every 3 weeks; or
b) 0.6-75 mg/kg or 45-5700 mg IV every 2 weeks; or
c) 0.3-38 mg/kg or 22-2900 mg IV weekly.

Embodiment 49: The method of Embodiment 21 wherein the antibody is AVE1642, or an antigen binding fragment thereof.

Embodiment 50: The method of Embodiment 49, wherein the AVE1642 antibody comprises a HCDR1 comprising the amino acid sequence of SEQ ID NO:25, a HCDR2 comprising the amino acid sequence of SEQ ID NO:76, a HCDR3 comprising the amino acid sequence of SEQ ID NO:27, a LCDR1 comprising the amino acid sequence of SEQ ID NO:28, a LCDR2 comprising the amino acid sequence of SEQ ID NO:29, and a LCDR3 comprising the amino acid sequence of SEQ ID NO:30.

Embodiment 51: The method of Embodiment 50, wherein the antibody comprises a heavy chain variable domain comprising SEQ ID NO:31 or 78 or 79, and a light chain variable domain comprising SEQ ID NO:32 or 80 or 81 or 82 or 83.

Embodiment 52: The method of Embodiment 51, wherein the antibody comprises a heavy chain variable domain comprising SEQ ID NO:78 and a light chain variable domain comprising SEQ ID NO:80 or 81 or 82 or 83.

Embodiment 53: The method of Embodiment 51, wherein the antibody comprises the light chain variable domain comprising SEQ ID NO:80.

Embodiment 54: The method of Embodiment 51, wherein the antibody comprises the light chain variable domain comprising SEQ ID NO:81.

Embodiment 55: The method of Embodiment 51, wherein the antibody comprises the light chain variable domain comprising SEQ ID NO:82.

Embodiment 56: The method of Embodiment 51, wherein the antibody comprises the light chain variable domain comprising SEQ ID NO:83.

Embodiment 57: The method of any of Embodiments 49-56 wherein the AVE1642 is dosed at:
a) 1-60 mg/kg or 75-4500 mg IV every 3 weeks; or
b) 0.6-40 mg/kg or 45-3000 mg IV every 2 weeks; or
c) 0.3-20 mg/kg or 22-1500 mg IV weekly.

Embodiment 58: The method of any of Embodiments 49-57, wherein the dose of the AVE1642 antibody is 1-10 mg/kg.

Embodiment 59: The method of Embodiment 58, wherein the wherein the dose of the AVE1642 antibody is 1-5 mg/kg.

Embodiment 60: The method of Embodiment 59, wherein the dose of the AVE1642 antibody is about 1 mg/kg, or about 2 mg/kg, or about 3 mg/kg, or about 4 mg/kg, or about 5 mg/kg.

Embodiment 61: The method of any of Embodiments 49-60, wherein the dose of the AVE1642 antibody is administered every 1, 2, 3, 4, or 5 weeks (i.e., QW, Q2W, Q3W, Q4W, or Q5W).

Embodiment 62: The method of Embodiment 61, wherein the AVE1642 is dosed at:
a) 1-5 mg/kg or 75-375 mg IV Q3W; or
b) 0.6-4 mg/kg or 45-300 mg IV Q2W; or
c) 0.3-3 mg/kg or 22-225 mg IV QW.

Embodiment 63: The method of any of Embodiments 49-62, wherein the AVE1642 antibody further comprises a variant Fc region comprising mutations that substitute a methionine at position 428 with a leucine (Met428Leu) and substitute an asparagine at position 434 with a serine (Asn434Ser), wherein the amino acid substitution numbering is EU as in Kabat.

Embodiment 64: The method of Embodiment 62, wherein the therapeutically effective amount of the AVE1642 antibody is administered every 1, 2, 3, 4, 5, 6, 7, 8, 9, 10, 11, or 12 weeks (i.e., QW, Q2W, Q3W, Q4W, Q5W, Q6W, Q7W, Q8W, Q9W, Q10W, Q11W, or Q12W).

Embodiment 65: The method of Embodiment 21 wherein the antibody is BIIB022, or an antigen binding fragment thereof.

Embodiment 66: The method of Embodiment 65 wherein the BIIB022 is dosed at:
a) 1-75 mg/kg or 75-5700 mg IV every 3 weeks; or
b) 0.6-50 mg/kg; or 45-3800 mg IV every 2 weeks; or
c) 0.3-25 mg/kg or 22-1900 mg IV weekly.

Embodiment 67: The method of Embodiment 21 wherein said IGF-1R inhibitor antibody, or an antigen binding fragment thereof, comprises a pair of (i) a heavy chain and (ii) a light chain, chosen from:
a heavy chain comprising the amino acid sequence of SEQ ID NO:7 and a light chain comprising the amino acid sequence SEQ ID NO:8;
a heavy chain comprising the amino acid sequence of SEQ ID NO:15 and a light chain comprising the amino acid sequence SEQ ID NO:16;
a heavy chain comprising the amino acid sequence of SEQ ID NO:23 and a light chain comprising the amino acid sequence SEQ ID NO:24;
a heavy chain comprising the amino acid sequence of SEQ ID NO:31, 78 or 79 and a light chain comprising the amino acid sequence SEQ ID NO:32, 80, 81, 82 or 83;
a heavy chain comprising the amino acid sequence of SEQ ID NO:31 and a light chain comprising the amino acid sequence SEQ ID NO:32;
a heavy chain comprising the amino acid sequence of SEQ ID NO:31 and a light chain comprising the amino acid sequence SEQ ID NO:80;
a heavy chain comprising the amino acid sequence of SEQ ID NO:31 and a light chain comprising the amino acid sequence SEQ ID NO:81;
a heavy chain comprising the amino acid sequence of SEQ ID NO:31 and a light chain comprising the amino acid sequence SEQ ID NO:82;
a heavy chain comprising the amino acid sequence of SEQ ID NO:31 and a light chain comprising the amino acid sequence SEQ ID NO:83;
a heavy chain comprising the amino acid sequence of SEQ ID NO:78 and a light chain comprising the amino acid sequence SEQ ID NO:32;
a heavy chain comprising the amino acid sequence of SEQ ID NO:78 and a light chain comprising the amino acid sequence SEQ ID NO:80;
a heavy chain comprising the amino acid sequence of SEQ ID NO:78 and a light chain comprising the amino acid sequence SEQ ID NO:81;
a heavy chain comprising the amino acid sequence of SEQ ID NO:78 and a light chain comprising the amino acid sequence SEQ ID NO:82;
a heavy chain comprising the amino acid sequence of SEQ ID NO:78 and a light chain comprising the amino acid sequence SEQ ID NO:83;
a heavy chain comprising the amino acid sequence of SEQ ID NO:79 and a light chain comprising the amino acid sequence SEQ ID NO:32;
a heavy chain comprising the amino acid sequence of SEQ ID NO:79 and a light chain comprising the amino acid sequence SEQ ID NO:80;
a heavy chain comprising the amino acid sequence of SEQ ID NO:79 and a light chain comprising the amino acid sequence SEQ ID NO:81;
a heavy chain comprising the amino acid sequence of SEQ ID NO:79 and a light chain comprising the amino acid sequence SEQ ID NO:82;
a heavy chain comprising the amino acid sequence of SEQ ID NO:79 and a light chain comprising the amino acid sequence SEQ ID NO:83;
a heavy chain comprising the amino acid sequence of SEQ ID NO:39 and a light chain comprising the amino acid sequence SEQ ID NO:40;
a heavy chain comprising the amino acid sequence of SEQ ID NO:47 and a light chain comprising the amino acid sequence SEQ ID NO:48;
a heavy chain comprising the amino acid sequence of SEQ ID NO:55 and a light chain comprising the amino acid sequence SEQ ID NO:56;
a heavy chain comprising the amino acid sequence of SEQ ID NO:63 and a light chain comprising the amino acid sequence SEQ ID NO:64;
a heavy chain comprising the amino acid sequence of SEQ ID NO:65 and a light chain comprising the amino acid sequence SEQ ID NO:66; and
a heavy chain comprising the amino acid sequence of SEQ ID NO:73 and a light chain comprising the amino acid sequence SEQ ID NO:74.

Embodiment 68: The method of any of Embodiments 1-13 wherein said IGF-1R inhibitor is a small molecule, or a salt or polymorph thereof.

Embodiment 69: The method of Embodiment 68 wherein said IGF-1R inhibitor, or salt or polymorph thereof, is dosed orally or intravenously.

Embodiment 70: The method of either of Embodiments 68 and 69 wherein said IGF-1R inhibitor, or salt or polymorph thereof, is chosen from linsitinib, picropodophyllin, BMS-754807, BMS-536924, BMS-554417, GSK1838705A, GSK1904529A, NVP-AEW541, NVP-ADW742, GTx-134, AG1024, KW-2450, PL-2258, NVP-AEW541, NSM-18, AZD3463, AZD9362, BI885578, BI893923, TT-100, XL-228, and A-928605, or a salt or polymorph of any of the foregoing.

Embodiment 71: The method of Embodiment 70 wherein the IGF-1R inhibitor is linsitinib, or a salt or polymorph thereof.

Embodiment 72: The method of Embodiment 71 wherein the linsitinib is dosed at:
a) 10-750 mg orally once daily continuous dosing or 10-1500 mg/day for once daily intermittent dosing (for up to 7 days of every 14 days); or
b) 6-500 mg orally twice daily continuous dosing or 6-1000 mg for twice daily intermittent dosing (for up to 7 days of every 14 days); or
c) 3-250 mg orally three-times daily continuous dosing or 3-500 mg for three-times daily intermittent dosing (for up to 7 days of every 14 days).

Embodiment 73: The method of Embodiment 70 wherein the IGF-1R inhibitor is picropodophyllin, or a salt or polymorph thereof.

Embodiment 74: The method of Embodiment 73 wherein the picropodophyllin is dosed:
a) orally once daily at 20-2000 mg; or
b) orally twice daily at 13-1400 mg; or
c) orally three times daily at 6-700 mg.

Embodiment 75: The method of Embodiment 70 wherein the IGF-1R inhibitor is BMS-754807, or a salt or polymorph thereof.

Embodiment 76: The method of Embodiment 75 wherein the BMS-754807 is dosed:
a) once daily at 5-600 mg orally; or
b) twice daily at 3-400 mg orally; or
c) three times daily at 1-200 mg.

Embodiment 77: The method of Embodiment 70 wherein the IGF-1R inhibitor is BMS-536924, or a salt or polymorph thereof.

Embodiment 78: The method of Embodiment 70 wherein the IGF-1R inhibitor is BMS-554417, or a salt or polymorph thereof.

Embodiment 79: The method of Embodiment 70 wherein the IGF-1R inhibitor is GSK1838705A, or a salt or polymorph thereof.

Embodiment 80: The method of Embodiment 70 wherein the IGF-1R inhibitor is GSK1904529A, or a salt or polymorph thereof.

Embodiment 81: The method of Embodiment 70 wherein the IGF-1R inhibitor is NVP-AEW541, or a salt or polymorph thereof.

Embodiment 82: The method of Embodiment 70 wherein the IGF-1R inhibitor is NVP-ADW742, or a salt or polymorph thereof.

Embodiment 83: The method of Embodiment 70 wherein the IGF-1R inhibitor is GTx-134, or a salt or polymorph thereof.

Embodiment 84: The method of Embodiment 70 wherein the IGF-1R inhibitor is AG1024, or a salt or polymorph thereof.

Embodiment 85: The method of Embodiment 70 wherein the IGF-1R inhibitor is PL-2258, or a salt or polymorph thereof.

Embodiment 86: The method of Embodiment 70 wherein the IGF-1R inhibitor is NVP-AEW541, or a salt or polymorph thereof.

Embodiment 87: The method of Embodiment 70 wherein the IGF-1R inhibitor is NSM-18, or a salt or polymorph thereof.

Embodiment 88: The method of Embodiment 70 wherein the IGF-1R inhibitor is AZD3463, or a salt or polymorph thereof.

Embodiment 89: The method of Embodiment 70 wherein the IGF-1R inhibitor is AZD9362, or a salt or polymorph thereof.

Embodiment 90: The method of Embodiment 70 wherein the IGF-1R inhibitor is BI885578, or a salt or polymorph thereof.

Embodiment 91: The method of Embodiment 70 wherein the IGF-1R inhibitor is BI893923, or a salt or polymorph thereof.

Embodiment 92: The method of Embodiment 70 wherein the IGF-1R inhibitor is TT-100, or a salt or polymorph thereof.

Embodiment 93: The method of Embodiment 70 wherein the IGF-1R inhibitor is XL-228, or a salt or polymorph thereof.

Embodiment 94: The method of Embodiment 70 wherein the IGF-1R inhibitor is A-928605, or a salt or polymorph thereof.

Embodiment 95: The method of any of Embodiments 77-94 wherein the IGF-1R inhibitor is dosed:
a) once daily at 1-2000 mg orally; or
b) twice daily at 0.6-1400 mg orally; or
c) three times daily at 0.3-700 mg orally.

Embodiment 96: The method of Embodiment 70 wherein the IGF-1R inhibitor is KW-2450, or a salt or polymorph thereof.

Embodiment 97: The method of Embodiment 96 wherein the KW-2450 is dosed:
a) once daily at 1-100 mg orally; or
b) twice daily at 0.6-70 mg orally; or
c) three times daily at 0.3-30 mg orally.

Embodiment 98: The method of any of Embodiments 1-97, comprising the concurrent or proximal administration of an additional therapeutic agent for the treatment of idiopathic orbital inflammation (IOI).

Embodiment 99: The method of Embodiment 98, wherein the additional therapeutic agent is a compound chosen from: a corticosteroid; rituximab or another anti-CD20 antibody; tocilizumab of another anti-IL-6 antibody; or methotrexate.

Embodiment 100: The method of any of Embodiments 1-99, wherein the treatment is efficacious for a least 4 weeks beyond the last administered dose.

Embodiment 101: The method of Embodiment 100, wherein the treatment is efficacious for a least 6 weeks beyond the last administered dose.

Embodiment 102: The method of Embodiment 100, wherein the treatment is efficacious for a least 8 weeks beyond the last administered dose.

Embodiment 103: The method of Embodiment 100, wherein the treatment is efficacious for a least 20 weeks beyond the last administered dose.

Also provided herein are pharmaceutical compositions for the treatment of IOI comprising an IGF-1R inhibitor.

Embodiment 104: A pharmaceutical composition comprising an amount of an insulin-like growth factor 1 receptor (IGF-1R) inhibitor effective for treating or reducing one or more effects of idiopathic orbital inflammation (IOI), or one or more symptoms thereof, in a subject with IOI.

Embodiment 105: The pharmaceutical composition of Embodiment 104, wherein the IOI is classified histopathologically as one of classic, granulomatous, sclerosing, and not otherwise specified (NOS), and/or is classified based on localization as one or more of diffuse, extraocular muscle, lacrimal gland, optic nerve, sclera and not otherwise specified (NOS).

Embodiment 106: The pharmaceutical composition of Embodiment 105, wherein the IOI is classic IOI.

Embodiment 107: the pharmaceutical composition of Embodiment 105, wherein the IOI is granulomatous IOI.

Embodiment 108: the pharmaceutical composition of Embodiment 105, wherein the IOI is sclerosing IOI.

Embodiment 109: the pharmaceutical composition of any of Embodiments 104-108, wherein the IOI is diffuse IOI.

Embodiment 110: the pharmaceutical composition of any of Embodiments 104-108, wherein the IOI is localized to the extraocular muscle.

Embodiment 111: the pharmaceutical composition of any of Embodiments 104-108, wherein the IOI is localized to the lacrimal gland.

Embodiment 112: the pharmaceutical composition of any of Embodiments 104-108, wherein the IOI is localized to the optic nerve.

Embodiment 113: the pharmaceutical composition of any of Embodiments 104-108, wherein the IOI is localized to the sclera.

Embodiment 114: the pharmaceutical composition of any of Embodiments 104-108, wherein the IOI is NOS.

Embodiment 115: the pharmaceutical composition of any of Embodiments 104-114, wherein the pharmaceutical composition comprising an amount of an insulin like growth factor-I receptor (IGF-1R) inhibitor is therapeutically effective:
for treating or reducing the severity of IOI or a symptom thereof;
for reducing eyelid edema;
for reducing ptosis;
for reducing proptosis;
for reducing globe displacement;
for reducing impairment of eye motility;
for reducing disease progression, e.g., involving one more of the lacrimal gland, the superior rectus muscle, the lateral rectus muscle, and the inferomedial orbit;
for reducing nongranulomatous, chronic inflammation;
for reducing sclerosing inflammation; and
for reducing nonspecific granulomatous inflammation

Embodiment 116: The pharmaceutical composition of Embodiment 104-115, wherein the IGF-1R inhibitor is an antibody, or an antigen binding fragment thereof, or a small molecule or a salt or polymorph thereof.

Embodiment 117: The pharmaceutical composition of Embodiment 116, wherein said IGF-1R inhibitor is chosen from teprotumumab, ganitumab, figitumumab, MEDI-573, cixutumumab, dalotuzumab, robatumumab, AVE1642, BIIB022, xentuzumab, istiratumab, linsitinib, picropodophyllin, BMS-754807, BMS-536924, BMS-554417, GSK1838705A, GSK1904529A, NVP-AEW541, NVP-ADW742, GTx-134, AG1024, KW-2450, PL-2258, INSM-18, AZD3463, AZD9362, BI885578, BI893923, TT-100, XL-228, and A-928605.

Embodiment 118: The pharmaceutical composition of Embodiment 117, wherein said IGF-1R inhibitor is an antibody, or an antigen binding fragment thereof.

Embodiment 119: The pharmaceutical composition of Embodiment 118, wherein said IGF-1R inhibitor is a human, chimeric human, or humanized monoclonal antibody, or an antigen binding fragment thereof, suitable for human therapy.

Embodiment 120: The pharmaceutical composition of Embodiment 119, comprising an antibody or antigen binding fragment thereof, is formulated to be administered intradermally, subcutaneously (SC), intravenously (IV), or by inhalation.

Embodiment 121: The pharmaceutical composition of Embodiment 120, comprising the antibody or antigen binding fragment thereof, is formulated to be administered intravenously (IV) or subcutaneously (SC).

Embodiment 122: The pharmaceutical composition of Embodiment 121, comprising the antibody or antigen binding fragment thereof, formulated to be administered IV.

Embodiment 123: The pharmaceutical composition of Embodiment 122, comprising the IGF-1R inhibitor, is formulated to be administered by IV infusion to the subject.

Embodiment 124: The pharmaceutical composition of any of Embodiments 118-121 wherein said antibody is chosen from teprotumumab, ganitumab, figitumumab, MEDI-573, cixutumumab, dalotuzumab, robatumumab, AVE1642, BIIB022, xentuzumab, and istiratumab, or an antigen binding fragment of any of the foregoing.

Embodiment 125: The pharmaceutical composition of any of Embodiments 104-124, wherein the IGF-1R inhibitor is an antibody, or antigen binding fragment thereof, that has a binding affinity (K_{D}) of 10⁻⁸ M or less for the IGF-1R.

Embodiment 126: The pharmaceutical composition of Embodiment 125, wherein the antibody, or antigen binding fragment thereof, has a binding affinity (K_{D}) of 10⁻³ to 10⁻⁹ M for the IGF-1R.

Embodiment 127: The pharmaceutical composition of any of Embodiments 104-126, wherein the antibody, or antigen binding fragment thereof, has an IC50 values for the IGF1 and IGF2 to IGF-1R of no more than 2 nM.

Embodiment 128: The pharmaceutical composition of any of Embodiments 104-127, wherein the IGF-1R inhibitor is an antibody, or an antigen binding fragment thereof, and comprises a heavy chain comprising CDR1, CDR2, and CDR3 and a light chain comprising CDR1, CDR2 and CDR3, wherein the heavy chain CDR1, CDR2, and CDR3 amino acid sequences and light chain CDR1, CDR2, and CDR3 amino acid sequences are at least 90% identical to (i) the amino acid sequences of SEQ ID NOs:83-89, respectively; or (ii) the amino acid sequences of SEQ ID NOs:84, 92, 86, 87, 93, and 89, respectively.

Embodiment 129: The pharmaceutical composition of Embodiment 128 wherein the antibody, or an antigen binding fragment thereof, comprises: (i) heavy chain CDR1, CDR2, and CDR3 amino acid sequences and light chain CDR1, CDR2, and CDR3 amino acid sequences as set forth in SEQ ID NOs: 83-89, respectively; or (ii) heavy chain CDR1, CDR2, and CDR3 amino acid sequences and light chain CDR1, CDR2, and CDR3 amino acid sequences as set forth in SEQ ID NOs: 83, 92, 86, 87, 93, and 89, respectively.

Embodiment 130: The pharmaceutical composition of any of Embodiments 104-129, wherein the IGF-1R inhibitor is an antibody, or an antigen binding fragment thereof, and comprises: (i) a heavy chain variable region having at least 80% sequence identity to the amino acid sequence of SEQ ID NO: 90 and a light chain variable region having at least 80% sequence identity to the amino acid sequence of SEQ ID NO: 91; or (ii) a heavy chain variable region having at least 80% sequence identity to the amino acid sequence of SEQ ID NO: 94 and a light chain variable region having at least 80% sequence identity to the amino acid sequence of SEQ ID NO: 95.

Embodiment 131: The pharmaceutical composition of Embodiment 130 wherein the antibody is teprotumumab, or an antigen binding fragment thereof.

Embodiment 132: The pharmaceutical composition of Embodiment 131, wherein the IGF-1R inhibitor is teprotumumab, formulated for administration:
a) 1-60 mg/kg or 75-4500 mg IV every 3 weeks; or
b) 0.6-40 mg/kg or 45-3000 mg IV every 2 weeks; or
c) 0.3-20 mg/kg; or 22-1500 mg IV weekly.

Embodiment 133: The pharmaceutical composition of Embodiment 131, wherein the IGF-1R inhibitor is an antibody, or antigen binding fragment thereof, and is formulated to be administered at a dosage about 1 mg/kg to about 5 mg/kg antibody as a first dose.

Embodiment 134: The pharmaceutical composition of Embodiment 131, wherein the IGF-1R inhibitor is an antibody, or antigen binding fragment thereof, and is formulated to be administered at a dosage about 5 mg/kg to about 10 mg/kg antibody as a first dose.

Embodiment 135: The pharmaceutical composition of any of Embodiments 133-134, wherein the IGF-1R inhibitor is an antibody, or antigen binding fragment thereof, formulated to be administered at a dosage about 5 mg/kg to about 20 mg/kg antibody in subsequent doses.

Embodiment 136: The pharmaceutical composition of Embodiment 135, wherein the IGF-1R inhibitor is an antibody, or antigen binding fragment thereof, formulated to be administered at a dosage about 10 mg/kg as a first dose; and about 20 mg/kg antibody in subsequent doses.

Embodiment 137: The pharmaceutical composition of any of Embodiments 133-136, wherein subsequent doses are administered every three weeks for at least 21 weeks.

Embodiment 138: The pharmaceutical composition of Embodiment 124, wherein the antibody is ganitumab, or an antigen binding fragment thereof.

Embodiment 139: The pharmaceutical composition of Embodiment 138, wherein the IGF-1R inhibitor is ganitumab, formulated for administration:
a) 1-60 mg/kg or 75-4500 mg IV every 3 weeks; or
b) 0.6-40 mg/kg or 45-3000 mg IV every 2 weeks; or
c) 0.3-20 mg/kg; or 22-1500 mg IV weekly.

Embodiment 140: The pharmaceutical composition of Embodiment 124 wherein the antibody is figitumumab, or an antigen binding fragment thereof.

Embodiment 141: The pharmaceutical composition of Embodiment 140, wherein the IGF-1R inhibitor is figitumumab, formulated for administration:
a) 1-60 mg/kg or 75-4500 mg IV every 3 weeks; or
b) 0.6-40 mg/kg or 45-3000 mg IV every 2 weeks; or
c) 0.3-20 mg/kg or 22-1500 mg IV weekly.

Embodiment 142: The pharmaceutical composition of Embodiment 124 wherein the antibody is cixutumumab, or an antigen binding fragment thereof.

Embodiment 143: The pharmaceutical composition of Embodiment 142, wherein the IGF-1R inhibitor is cixutumumab, formulated for administration:
a) 1-45 mg/kg or 75-3400 mg IV every 3 weeks; or
b) 0.6-30 mg/kg or 45-2300 mg IV every 2 weeks; or
c) 0.3-15 mg/kg or 22-1200 mg IV weekly.

Embodiment 144: The pharmaceutical composition of Embodiment 124 wherein the antibody is dalotuzumab, or an antigen binding fragment thereof.

Embodiment 145: The pharmaceutical composition of Embodiment 144, wherein the IGF-1R inhibitor is dalotuzumab, formulated for administration:
a) 1-90 mg/kg or 75-6800 mg IV every 3 weeks; or
b) 0.6-60 mg/kg or 45-4500 mg IV every 2 weeks; or
c) 0.3-30 mg/kg or 22-2300 mg IV weekly.

Embodiment 146: The pharmaceutical composition of Embodiment 124 wherein the antibody is robatumumab, or an antigen binding fragment thereof.

Embodiment 147: The pharmaceutical composition of Embodiment 146, wherein the IGF-1R inhibitor is robatumumab, formulated for administration:
a) 1-75 mg/kg or 75-5700 mg IV every 3 weeks; or
b) 0.6-50 mg/kg or 45-3800 mg IV every 2 weeks; or
c) 0.3-25 mg/kg or 22-1900 mg IV weekly.

Embodiment 148: The pharmaceutical composition of Embodiment 124 wherein the antibody is xentuzumab, or an antigen binding fragment thereof.

Embodiment 149: The pharmaceutical composition of Embodiment 148, wherein the IGF-1R inhibitor is xentuzumab, formulated for administration:
a) 1-112 mg/kg or 75-8400 mg IV every 3 weeks; or
b) 0.6-75 mg/kg or 45-5700 mg IV every 2 weeks; or
c) 0.3-38 mg/kg or 22-2900 mg IV weekly.

Embodiment 150: The pharmaceutical composition of Embodiment 124 wherein the antibody is istiratumab, or an antigen binding fragment thereof.

Embodiment 151: The pharmaceutical composition of Embodiment 150, wherein the IGF-1R inhibitor is istiratumab, formulated for administration:
a) 1-112 mg/kg or 75-8400 mg IV every 3 weeks; or
b) 0.6-75 mg/kg or 45-5700 mg IV every 2 weeks; or
c) 0.3-38 mg/kg or 22-2900 mg IV weekly.

Embodiment 152: The pharmaceutical composition of Embodiment 124 wherein the antibody is AVE1642, or an antigen binding fragment thereof.

Embodiment 153: The pharmaceutical composition of Embodiment 152, wherein the AVE1642 antibody comprises a HCDR1 comprising the amino acid sequence of SEQ ID NO:25, a HCDR2 comprising the amino acid sequence of SEQ ID NO:76, a HCDR3 comprising the amino acid sequence of SEQ ID NO:27, a LCDR1 comprising the amino acid sequence of SEQ ID NO:28, a LCDR2 comprising the amino acid sequence of SEQ ID NO:29, and a LCDR3 comprising the amino acid sequence of SEQ ID NO:30.

Embodiment 154: The pharmaceutical composition of Embodiment 153, wherein the antibody comprises a heavy chain variable domain comprising SEQ ID NO:31 or 78 or 79, and a light chain variable domain comprising SEQ ID NO:32 or 80 or 81 or 82 or 83.

Embodiment 155: The pharmaceutical composition of Embodiment 154, wherein the antibody comprises a heavy chain variable domain comprising SEQ ID NO:78 and a light chain variable domain comprising SEQ ID NO:80 or 81 or 82 or 83.

Embodiment 156: The pharmaceutical composition of Embodiment 154, wherein the antibody comprises the light chain variable domain comprising SEQ ID NO:80.

Embodiment 157: The pharmaceutical composition of Embodiment 154, wherein the antibody comprises the light chain variable domain comprising SEQ ID NO:81.

Embodiment 158: The pharmaceutical composition of Embodiment 154, wherein the antibody comprises the light chain variable domain comprising SEQ ID NO:82.

Embodiment 159: The pharmaceutical composition of Embodiment 154, wherein the antibody comprises the light chain variable domain comprising SEQ ID NO:83.

Embodiment 160: The pharmaceutical composition of any of Embodiments 152-159, wherein the IGF-1R inhibitor is AVE1642, formulated for administration:
a) 1-60 mg/kg or 75-4500 mg IV every 3 weeks; or
b) 0.6-40 mg/kg or 45-3000 mg IV every 2 weeks; or
c) 0.3-20 mg/kg or 22-1500 mg IV weekly.

Embodiment 161: The pharmaceutical composition of any of Embodiments 152-160, formulated to contain a dose of the AVE1642 antibody to be 1-10 mg/kg.

Embodiment 162: The pharmaceutical composition of Embodiment 161, formulated to contain a dose of the AVE1642 antibody to be 1-5 mg/kg.

Embodiment 163: The pharmaceutical composition of Embodiment 162, formulated to contain a dose of the AVE1642 antibody to be about 1 mg/kg, or about 2 mg/kg, or about 3 mg/kg, or about 4 mg/kg, or about 5 mg/kg.

Embodiment 164: The pharmaceutical composition of any of Embodiments 152-163, wherein the IGF-1R inhibitor is AVE1642 antibody, formulated to be administered every 1, 2, 3, 4, or 5 weeks (i.e., QW, Q2W, Q3W, Q4W, or Q5W).

Embodiment 165: The pharmaceutical composition of Embodiment 164, wherein the IGF-1R inhibitor is AVE1642, formulated to be dosed at:
a) 1-5 mg/kg or 75-375 mg IV Q3W; or
b) 0.6-4 mg/kg or 45-300 mg IV Q2W; or
c) 0.3-3 mg/kg or 22-225 mg IV QW.

Embodiment 166: The pharmaceutical composition of any of Embodiments 152-165, wherein the AVE1642 antibody further comprises a variant Fc region comprising mutations that substitute a methionine at position 428 with a leucine (Met428Leu) and substitute an asparagine at position 434 with a serine (Asn434Ser), wherein the amino acid substitution numbering is EU as in Kabat.

Embodiment 167: The pharmaceutical composition of Embodiment 165, formulated to contain the therapeutically effective amount of the AVE1642 antibody, is administered every 1, 2, 3, 4, 5, 6, 7, 8, 9, 10, 11, or 12 weeks (i.e., QW, Q2W, Q3W, Q4W, Q5W, Q6W, Q7W, Q8W, Q9W, Q10W, Q11W, or Q12W).

Embodiment 168: The pharmaceutical composition of Embodiment 124 wherein the antibody is BIIB022, or an antigen binding fragment thereof.

Embodiment 169: The pharmaceutical composition of Embodiment 168, wherein the IGF-1R inhibitor is BIIB022, formulated for administration at:
a) 1-75 mg/kg or 75-5700 mg IV every 3 weeks; or
b) 0.6-50 mg/kg; or 45-3800 mg IV every 2 weeks; or
c) 0.3-25 mg/kg or 22-1900 mg IV weekly.

Embodiment 170: The pharmaceutical composition of Embodiment 124 wherein said IGF-1R inhibitor antibody, or an antigen binding fragment thereof, comprises a pair of (i) a heavy chain and (ii) a light chain, chosen from:
a heavy chain comprising the amino acid sequence of SEQ ID NO:7 and a light chain comprising the amino acid sequence SEQ ID NO:8;
a heavy chain comprising the amino acid sequence of SEQ ID NO:15 and a light chain comprising the amino acid sequence SEQ ID NO:16;
a heavy chain comprising the amino acid sequence of SEQ ID NO:23 and a light chain comprising the amino acid sequence SEQ ID NO:24;
a heavy chain comprising the amino acid sequence of SEQ ID NO:31, 78 or 79 and a light chain comprising the amino acid sequence SEQ ID NO:32, 80, 81, 82 or 83;
a heavy chain comprising the amino acid sequence of SEQ ID NO:31 and a light chain comprising the amino acid sequence SEQ ID NO:32;
a heavy chain comprising the amino acid sequence of SEQ ID NO:31 and a light chain comprising the amino acid sequence SEQ ID NO:80;
a heavy chain comprising the amino acid sequence of SEQ ID NO:31 and a light chain comprising the amino acid sequence SEQ ID NO:81;
a heavy chain comprising the amino acid sequence of SEQ ID NO:31 and a light chain comprising the amino acid sequence SEQ ID NO:82;
a heavy chain comprising the amino acid sequence of SEQ ID NO:31 and a light chain comprising the amino acid sequence SEQ ID NO:83;
a heavy chain comprising the amino acid sequence of SEQ ID NO:78 and a light chain comprising the amino acid sequence SEQ ID NO:32;
a heavy chain comprising the amino acid sequence of SEQ ID NO:78 and a light chain comprising the amino acid sequence SEQ ID NO:80;
a heavy chain comprising the amino acid sequence of SEQ ID NO:78 and a light chain comprising the amino acid sequence SEQ ID NO:81;
a heavy chain comprising the amino acid sequence of SEQ ID NO:78 and a light chain comprising the amino acid sequence SEQ ID NO:82;
a heavy chain comprising the amino acid sequence of SEQ ID NO:78 and a light chain comprising the amino acid sequence SEQ ID NO:83;
a heavy chain comprising the amino acid sequence of SEQ ID NO:79 and a light chain comprising the amino acid sequence SEQ ID NO:32;
a heavy chain comprising the amino acid sequence of SEQ ID NO:79 and a light chain comprising the amino acid sequence SEQ ID NO:80;
a heavy chain comprising the amino acid sequence of SEQ ID NO:79 and a light chain comprising the amino acid sequence SEQ ID NO:81;
a heavy chain comprising the amino acid sequence of SEQ ID NO:79 and a light chain comprising the amino acid sequence SEQ ID NO:82;
a heavy chain comprising the amino acid sequence of SEQ ID NO:79 and a light chain comprising the amino acid sequence SEQ ID NO:83;
a heavy chain comprising the amino acid sequence of SEQ ID NO:39 and a light chain comprising the amino acid sequence SEQ ID NO:40;
a heavy chain comprising the amino acid sequence of SEQ ID NO:47 and a light chain comprising the amino acid sequence SEQ ID NO:48;
a heavy chain comprising the amino acid sequence of SEQ ID NO:55 and a light chain comprising the amino acid sequence SEQ ID NO:56;
a heavy chain comprising the amino acid sequence of SEQ ID NO:63 and a light chain comprising the amino acid sequence SEQ ID NO:64;
a heavy chain comprising the amino acid sequence of SEQ ID NO:65 and a light chain comprising the amino acid sequence SEQ ID NO:66; and
a heavy chain comprising the amino acid sequence of SEQ ID NO:73 and a light chain comprising the amino acid sequence SEQ ID NO:74.

Embodiment 171: The pharmaceutical composition of Embodiment 116 wherein said IGF-1R inhibitor is a small molecule, or a salt or polymorph thereof.

Embodiment 172: The pharmaceutical composition of Embodiment 171, comprising said IGF-1R inhibitor, or salt or polymorph thereof, is formulated to be dosed orally or intravenously.

Embodiment 173: The pharmaceutical composition of either of Embodiments 171 and 172 wherein said IGF-1R inhibitor, or salt or polymorph thereof, is chosen from linsitinib, picropodophyllin, BMS-754807, BMS-536924, BMS-554417, GSK1838705A, GSK1904529A, NVP-AEW541, NVP-ADW742, GTx-134, AG1024, KW-2450, PL-2258, NVP-AEW541, NSM-18, AZD3463, AZD9362, BI885578, BI893923, TT-100, XL-228, and A-928605, or a salt or polymorph of any of the foregoing.

Embodiment 174: The pharmaceutical composition of Embodiment 173 wherein the IGF-1R inhibitor is linsitinib, or a salt or polymorph thereof.

Embodiment 175: The pharmaceutical composition of Embodiment 174, wherein the IGF-1R inhibitor is linsitinib, formulated for administration:
a) 10-750 mg orally once daily continuous dosing or 10-1500 mg/day for once daily intermittent dosing (for up to 7 days of every 14 days); or
b) 6-500 mg orally twice daily continuous dosing or 6-1000 mg for twice daily intermittent dosing (for up to 7 days of every 14 days); or
c) 3-250 mg orally three-times daily continuous dosing or 3-500 mg for three-times daily intermittent dosing (for up to 7 days of every 14 days).

Embodiment 176: The pharmaceutical composition of Embodiment 173, wherein the IGF-1R inhibitor is picropodophyllin, or a salt or polymorph thereof.

Embodiment 177: The pharmaceutical composition of Embodiment 176, wherein the IGF-1R inhibitor is picropodophyllin, formulated for administration:
a) orally once daily at 20-2000 mg; or
b) orally twice daily at 13-1400 mg; or
c) orally three times daily at 6-700 mg.

Embodiment 178: The pharmaceutical composition of Embodiment 173 wherein the IGF-1R inhibitor is BMS-754807, or a salt or polymorph thereof.

Embodiment 179: The pharmaceutical composition of Embodiment 179, wherein the IGF-1R inhibitor is BMS-754807, formulated for administration:
a) once daily at 5-600 mg orally; or
b) twice daily at 3-400 mg orally; or
c) three times daily at 1-200 mg.

Embodiment 180: The pharmaceutical composition of Embodiment 173 wherein the IGF-1R inhibitor is BMS-536924, or a salt or polymorph thereof.

Embodiment 181: The pharmaceutical composition of Embodiment 173 wherein the IGF-1R inhibitor is BMS-554417, or a salt or polymorph thereof.

Embodiment 182: The pharmaceutical composition of Embodiment 173 wherein the IGF-1R inhibitor is GSK1838705A, or a salt or polymorph thereof.

Embodiment 183: The pharmaceutical composition of Embodiment 173 wherein the IGF-1R inhibitor is GSK1904529A, or a salt or polymorph thereof.

Embodiment 184: The pharmaceutical composition of Embodiment 173 wherein the IGF-1R inhibitor is NVP-AEW541, or a salt or polymorph thereof.

Embodiment 185: The pharmaceutical composition of Embodiment 173 wherein the IGF-1R inhibitor is NVP-ADW742, or a salt or polymorph thereof.

Embodiment 186: The pharmaceutical composition of Embodiment 173 wherein the IGF-1R inhibitor is GTx-134, or a salt or polymorph thereof.

Embodiment 187: The pharmaceutical composition of Embodiment 173 wherein the IGF-1R inhibitor is AG1024, or a salt or polymorph thereof.

Embodiment 188: The pharmaceutical composition of Embodiment 173 wherein the IGF-1R inhibitor is PL-2258, or a salt or polymorph thereof.

Embodiment 189: The pharmaceutical composition of Embodiment 173 wherein the IGF-1R inhibitor is NVP-AEW541, or a salt or polymorph thereof.

Embodiment 190: The pharmaceutical composition of Embodiment 173 wherein the IGF-1R inhibitor is NVP-AEW541, or a salt or polymorph thereof.

Embodiment 191: The pharmaceutical composition of Embodiment 173 wherein the IGF-1R inhibitor is AZD3463, or a salt or polymorph thereof.

Embodiment 192: The pharmaceutical composition of Embodiment 173 wherein the IGF-1R inhibitor is AZD9362, or a salt or polymorph thereof.

Embodiment 193: The pharmaceutical composition of Embodiment 173 wherein the IGF-1R inhibitor is BI885578, or a salt or polymorph thereof.

Embodiment 194: The pharmaceutical composition of Embodiment 173 wherein the IGF-1R inhibitor is BI893923, or a salt or polymorph thereof.

Embodiment 195: The pharmaceutical composition of Embodiment 173 wherein the IGF-1R inhibitor is TT-100, or a salt or polymorph thereof.

Embodiment 196: The pharmaceutical composition of Embodiment 173 wherein the IGF-1R inhibitor is XL-228, or a salt or polymorph thereof.

Embodiment 197: The pharmaceutical composition of Embodiment 173 wherein the IGF-1R inhibitor is A-928605, or a salt or polymorph thereof.

Embodiment 198: The pharmaceutical composition of any of Embodiments 180-197, wherein the IGF-1R inhibitor is a small molecule or a salt or polymorph thereof, formulated for administration:
a) once daily at 1-2000 mg orally; or
b) twice daily at 0.6-1400 mg orally; or
c) three times daily at 0.3-700 mg orally.

Embodiment 199: The pharmaceutical composition of Embodiment 173 wherein the IGF-1R inhibitor is KW-2450, or a salt or polymorph thereof.

Embodiment 200: The pharmaceutical composition of Embodiment 199, wherein the IGF-1R inhibitor is KW-2450 or a salt or polymorph there, formulated for administration:
a) once daily at 1-100 mg orally; or
b) twice daily at 0.6-70 mg orally; or
c) three times daily at 0.3-30 mg orally.

Embodiment 201: The pharmaceutical composition of any of Embodiments 104-200, comprising the concurrent or proximal administration of an additional therapeutic agent for the treatment of idiopathic orbital inflammation (TOO.

Embodiment 202: The pharmaceutical composition of Embodiment 201, wherein the additional therapeutic agent is a compound chosen from: a corticosteroid; rituximab or another anti-CD20 antibody; tocilizumab of another anti-IL-6 antibody; or methotrexate.

Embodiment 203: The pharmaceutical composition of any of Embodiments 104-202, wherein the treatment is efficacious for a least 4 weeks beyond the last administered dose.

Embodiment 204: The pharmaceutical composition of Embodiment 203, wherein the treatment is efficacious for a least 6 weeks beyond the last administered dose.

Embodiment 205: The pharmaceutical composition of Embodiment 203, wherein the treatment is efficacious for a least 8 weeks beyond the last administered dose.

Embodiment 206: The pharmaceutical composition of Embodiment 203, wherein the treatment is efficacious for a least 20 weeks beyond the last administered dose.

Also provided is a pharmaceutical composition as recited in any of Embodiments 104-205 for use in treating or reducing one or more effects of idiopathic orbital inflammation (IOI), or one or more symptoms thereof, as set forth in any of Embodiments 1-103.

Also provided is the use of a pharmaceutical composition comprising an effective amount of an insulin-like growth factor 1 receptor (IGF-1R) inhibitor, as recited in any of Embodiments 104-206, for use in treating or reducing one or more effects of idiopathic orbital inflammation (IOI), or one or more symptoms thereof, as set forth in any of Embodiments 1-103.

Also provided is an insulin-like growth factor 1 receptor (IGF-1R) inhibitor for use in the manufacture of a pharmaceutical composition (a medicament) as recited in any of Embodiments 104-206, for treating or reducing one or more effects of idiopathic orbital inflammation (IOI), or one or more symptoms thereof, as set forth in any of Embodiments 1-103.

Also Provided are the Following Embodiments.

Embodiment P1: A method of treating idiopathic orbital inflammation (IOD or one or more symptoms thereof comprising administering to a subject in need thereof a therapeutically effective amount of an inhibitor of insulin like growth factor 1 receptor (IGF-R).

Embodiment P2: The method of Embodiment P1, wherein the administration is by intradermal injection, subcutaneous injection, intravenous injection, or by inhalation.

Embodiment P3: The method of any of Embodiments P1-P2, wherein the antibody, or antigen binding fragment thereof, is administered at a dosage of about 1 mg/kg to about 5 mg/kg as a first dose.(this dosage is tepro specific—put later or cut)

Embodiment P4: The method of any of Embodiments P1-P2, wherein the antibody, or antigen binding fragment thereof, is administered at a dosage of about 5 mg/kg to about 10 mg/kg as a first dose.

Embodiment P5: The method of Embodiment P4, wherein the antibody, or antigen binding fragment thereof, is administered at a dosage of about 5 mg/kg to about 20 mg/kg in subsequent doses.

Embodiment P6: The method of Embodiment P4, wherein the antibody, or antigen binding fragment thereof, is administered at a dosage of about 10 mg/kg as a first dose; and about 20 mg/kg in subsequent doses.

Embodiment P7: The method of Embodiment P6, wherein the subsequent doses are administered every three weeks for at least 21 weeks.

Embodiment P8: The method of any of Embodiments P1-P7, wherein the antibody, or an antigen binding fragment thereof, has a binding affinity (K_{D}) of 10⁻⁸ M or less for IGF-1R.

Embodiment P9: The method of Embodiment P8, wherein the antibody, or an antigen binding fragment thereof, has a binding affinity (K_{D}) of 10⁻¹³ to 10⁻⁹ M for the IGF-1R.

Embodiment P10: The method of any of Embodiments P1-P7, wherein the antibody, or an antigen binding fragment thereof, has an IC50 value for the binding of IGF-I and IGF-II to IGF-IR of no more than about 2 nM.

Embodiment P11: The method of Embodiment P10, wherein the antibody, or an antigen binding fragment thereof, comprises a heavy chain comprising CDR1, CDR2, and CDR3 and a light chain comprising CDR1, CDR2 and CDR3, wherein the heavy chain CDR1, CDR2, and CDR3 amino acid sequences and light chain CDR1, CDR2, and CDR3 amino acid sequences are at least 90% identical to (i) the amino acid sequences of SEQ ID NOs:84-89, respectively; or (ii) the amino acid sequences of SEQ ID NOs:84, 92, 86, 87, 93, and 89 respectively.

Embodiment P12: The method of Embodiment P11, wherein the antibody, or an antigen binding fragment thereof, comprises: (i) heavy chain CDR1, CDR2, and CDR3 amino acid sequences and light chain CDR1, CDR2, and CDR3 amino acid sequences as set forth in SEQ ID NOs:84-89, respectively; or (ii) heavy chain CDR1, CDR2, and CDR3 amino acid sequences and light chain CDR1, CDR2, and CDR3 amino acid sequences as set forth in SEQ ID NOs:84, 92, 86, 87, 93, and 89, respectively.

Embodiment P13: The method of Embodiment P11, wherein the antibody, or an antigen binding fragment thereof, comprises: (i) a heavy chain variable region having at least 80% sequence identity to the amino acid sequence of SEQ ID NO: 90 and a light chain variable region having at least 80% sequence identity to the amino acid sequence of SEQ ID NO: 91; or (ii) a heavy chain variable region having at least 80% sequence identity to the amino acid sequence of SEQ ID NO: 94 and a light chain variable region having at least 80% sequence identity to the amino acid sequence of SEQ ID NO: 95.

Embodiment P14: The method of Embodiment P11, wherein the antibody, or an antigen binding fragment thereof, comprises: (i) a heavy chain variable region comprising the amino acid sequence of SEQ ID NO: 90 and a light chain variable region comprising the amino acid sequence of SEQ ID NO: 91; or (ii) a heavy chain variable region comprising the amino acid sequence of SEQ ID NO: 94 and a light chain variable region comprising the amino acid sequence of SEQ ID NO: 95.

Embodiment P15: The method of any of Embodiments P1-P14, wherein the antibody is teprotumumab.

Embodiment P16: The method of any of Embodiments P1-P14, wherein the antibody, or an antigen binding fragment thereof, is a human antibody, a monoclonal antibody, a human monoclonal antibody, a purified antibody, a diabody, a single-chain antibody, a multi-specific antibody, Fab, Fab′, F(ab′)2, Fv or scFv.

Embodiment P17: The method of any of Embodiments P1-P16, wherein the antibody, or an antigen binding fragment thereof, is administered in a pharmaceutical composition that additionally comprises a pharmaceutically acceptable diluent or excipient or carrier.

Embodiment P18: The method of Embodiment P17, additionally comprising administering, as part of the pharmaceutical composition or separately, one or more other pharmaceutically active compounds for the treatment of IOI.

Embodiment P19: The method of Embodiment P17, wherein the additionally comprising administering, as part of the pharmaceutical composition or separately, a compound chosen from: a corticosteroid; rituximab or another anti-CD20 antibody; tocilizumab or another anti-IL-6 antibody; or methotrexate.

Embodiment P20: The method of Embodiment P17, wherein the treatment is efficacious for at least 4 weeks beyond the last administered dose.

Embodiment P21: The method of Embodiment P17, wherein the treatment is efficacious for at least 6 weeks beyond the last administered dose.

Embodiment P22: The method of Embodiment P17, wherein the treatment is efficacious for at least 8 weeks beyond the last administered dose.

Embodiment P23: The method of Embodiment P17, wherein the treatment is efficacious for at least 20 weeks beyond the last administered dose.

### Definitions

The following terms shall be understood to have the meanings ascribed herein.

The term “about,” as used herein in relation to a numerical value x means x±10%.

The term “and/or” when used in a list of two or more items, means that any one of the listed items can be employed by itself or in combination with any one or more of the listed items. For example, the expression “A and/or B” is intended to mean either or both of A and B, i.e., A alone, B alone or A and B in combination. The expression “A, B and/or C” is intended to mean A alone, B alone, C alone, A and B in combination, A and C in combination, B and C in combination or A, B, and C in combination.

As used herein, the term “antibody” encompasses the various forms of antibodies, including but not limited to whole antibodies, monoclonal antibodies, antibody fragments, human antibodies, humanized antibodies, chimeric antibodies, and genetically engineered antibodies, as long as the characteristic properties such as specificity and IGF-IR inhibitory are retained.

As used herein, the terms “antigen binding fragment,” “fragment,” and “antibody fragment” are used interchangeably to refer to any fragment that comprises a portion of a full length antibody, generally at least the antigen binding portion or the variable region thereof. Examples of antibody fragments include, but are not limited to, diabodies, single-chain antibody molecules, multispecific antibodies, Fab, Fab′, F(ab′)₂, Fv or scFv. Further, the term “antibody” as used herein includes both antibodies and antigen binding fragments thereof. In addition, antibody fragments comprise single chain polypeptides having the characteristics of a VH chain, namely being able to assemble together with a VL chain or of a VL chain binding to IGF-IR, namely being able to assemble together with a VH chain to a functional antigen binding pocket and thereby providing the property of inhibiting the binding of IGF-I and IGF-II to IGF-IR.

The terms “binding to IGF-IR” or “specific binding to IGF-IR” are used interchangeably herein and mean the binding of the antibody to IGF-IR in an in vitro assay, preferably in a binding assay in which the antibody is bound to a surface and binding of IGF-IR is measured by Surface Plasmon Resonance (SPR). Binding means a binding affinity (K_{D}) of 10⁻⁸ M or less, preferably 10⁻¹³ to 10⁻⁹ M. Binding to IGF-IR can be investigated by a BIAcore assay (Pharmacia Biosensor AB, Uppsala, Sweden). The affinity of the binding is defined by the terms ka (rate constant for the association of the antibody from the antibody/antigen complex), kd (dissociation constant), and K_{D} (kd/ka). The antibodies used in the methods disclosed herein show a K_{D} of about 10⁻⁹ M or less.

The term “combination therapy” means the administration of two or more therapeutic agents to treat a therapeutic condition or disorder described in the present disclosure. Such administration encompasses co-administration of these therapeutic agents in a substantially simultaneous manner, such as in a single capsule having a fixed ratio of active ingredients or in multiple, separate capsules for each active ingredient. In addition, such administration also encompasses use of each type of therapeutic agent in a sequential manner. In either case, the treatment regimen will provide beneficial effects of the drug combination in treating the conditions or disorders described herein.

The terms “complementarity determining region,” “CDR,” “hypervariable region,” or “antigen-binding portion of an antibody” are used interchangeably herein and refer to the amino acid residues of an antibody which are responsible for antigen-binding. The hypervariable region comprises amino acid residues from the complementarity determining regions or CDRs. “Framework” or “FR” regions are those variable domain regions other than the hypervariable region residues as herein defined. Therefore, the light and heavy chains of an antibody comprise from N- to C-terminus the domains FR1, CDR1, FR2, CDR2, FR3, CDR3, and FR4. Especially, CDR3 of the heavy chain is the region which contributes most to antigen binding. CDR and FR regions are determined according to the standard definition of Kabat et al., Sequences of Proteins of Immunological Interest, 5th Ed. Public Health Service, National Institutes of Health, Bethesda, Md. (1991)) and/or those residues from a “hypervariable loop.”

The term “comprising” encompasses “including” as well as “consisting” e.g., a composition “comprising” X may consist exclusively of X or may include something additional e.g., X+Y.

The term “disease” as used herein is intended to be generally synonymous, and is used interchangeably with, the terms “disorder,” “syndrome,” and “condition” (as in medical condition), in that all reflect an abnormal condition of the human or animal body or of one of its parts that impairs normal functioning, is typically manifested by distinguishing signs and symptoms, and causes the human or animal to have a reduced duration or quality of life.

The term “human antibody” as used herein, is intended to include antibodies having variable and constant regions derived from human germline immunoglobulin sequences. The term “humanized antibody” as used herein refers to antibodies in which the framework or “complementarity determining regions” (CDR) have been modified to comprise the CDR of an immunoglobulin of different specificity as compared to that of the parent immunoglobulin. In a preferred embodiment, a murine CDR is grafted into the framework region of a human antibody to prepare the “humanized antibody.”

The term “inhibiting the binding of IGF-I and IGF-II to IGF-IR” as used herein refers to inhibiting the binding of I¹²⁵-labeled IGF-I or IGF-II to IGF-IR presented on the surface of cells in an in vitro assay. Inhibiting means an IC₅₀ value of 2 nM or lower.

The terms “monoclonal antibody” or “monoclonal antibody composition,” as used herein refer to a preparation of antibody molecules of a single amino acid composition. Accordingly, the term “human monoclonal antibody” refers to antibodies displaying a single binding specificity which have variable and constant regions derived from human germline immunoglobulin sequences. In one embodiment, the human monoclonal antibodies are produced by a hybridoma which includes a B cell obtained from a transgenic non-human animal, e.g., a transgenic mouse, having a genome comprising a human heavy chain transgene and a light human chain transgene fused to an immortalized cell.

The term “human antibody” as used herein, is intended to include antibodies having variable and constant regions derived from human germline immunoglobulin sequences. The term “humanized antibody” as used herein refers to antibodies in which the framework or “complementarity determining regions” (CDR) have been modified to comprise the CDR of an immunoglobulin of different specificity as compared to that of the parent immunoglobulin. In a preferred embodiment, a murine CDR is grafted into the framework region of a human antibody to prepare the “humanized antibody.”

The term “recombinant human antibody,” as used herein, is intended to include all human antibodies that are prepared, expressed, created or isolated by recombinant means, such as antibodies isolated from a host cell such as an SP2-0, NS0 or CHO cell or from an animal (e.g., a mouse) that is transgenic for human immunoglobulin genes or antibodies expressed using a recombinant expression vector transfected into a host cell. Such recombinant human antibodies have variable and constant regions derived from human germline immunoglobulin sequences in a rearranged form.

The term “variable region” (variable region of a light chain (VL), variable region of a heavy chain (VH)) as used herein denotes each of the pair of light and heavy chains which is involved directly in binding the antibody to the antigen.

The terms “subject” and “patient” are used interchangeably herein to mean all mammals including humans. Examples of subjects include, but are not limited to, humans, monkeys, dogs, cats, horses, cows, goats, sheep, pigs, and rabbits. In one embodiment, the subject or patient is a human. The terms “affected with a disease or disorder,” “afflicted with a disease or disorder,” or “having a disease or disorder” are used interchangeably herein and refer to a subject or patient with any disease, disorder, syndrome, or condition. No increased or decreased level of severity of the disorder is implied by the use of one the terms as compared to the other.

The word “substantially” does not exclude “completely” e.g., a composition which is “substantially free” from Y may be completely free from Y. Where necessary, the word “substantially” may be omitted.

The term “teprotumumab,” also known as RV-001 and R-1507 and currently marketed under the trade name TEPEZZA™, is a human monoclonal antibody that binds to insulin-like growth factor-1 receptor (IGF-1R). It has CAS number 1036734-93-6 and comprises a SEQ ID NO.S 84-91 disclosed herein (see, e.g., Table A). It comprises and may be referred to in the alternative throughout this disclosure as “Antibody 1.”

The term “therapeutically acceptable” refers to those compounds (or salts, prodrugs, tautomers, zwitterionic forms, etc.) which are suitable for use in contact with the tissues of patients without undue toxicity, irritation, and allergic response, are commensurate with a reasonable benefit/risk ratio, and are effective for their intended use.

The phrase “therapeutically effective” is intended to qualify the amount of active ingredients used in the treatment of a disease or disorder or on the effecting of a clinical endpoint.

The terms “treating,” “treatment,” and the like, as used herein, mean ameliorating a disease, so as to reduce, ameliorate, or eliminate its cause, its progression, its severity, or one or more of its symptoms, or otherwise beneficially alter the disease in a subject. Reference to “treating,” or “treatment” of a patient is intended to include prophylaxis. Treatment may also be preemptive in nature, i.e., it may include prevention of disease in a subject exposed to or at risk for the disease. Prevention of a disease may involve complete protection from disease, for example as in the case of prevention of infection with a pathogen, or may involve prevention of disease progression, for example from prediabetes to diabetes. For example, prevention of a disease may not mean complete foreclosure of any effect related to the diseases at any level, but instead may mean prevention of the symptoms of a disease to a clinically significant or detectable level. Prevention of diseases may also mean prevention of progression of a disease to a later stage of the disease.

The domains of variable human light and heavy chains have the same general structure and each domain comprises four framework (FR) regions whose sequences are widely conserved, connected by three “hypervariable regions” (or complementarity determining regions, CDRs). The framework regions adopt a β-sheet conformation and the CDRs may form loops connecting the β-sheet structure. The CDRs in each chain are held in their three-dimensional structure by the framework regions and form together with the CDRs from the other chain the antigen binding site. The antibody heavy and light chain CDR3 regions play an important role in the binding specificity/affinity of antibodies.

The terms “complementarity determining region,” “CDR,” “hypervariable region,” or “antigen-binding portion of an antibody” are used interchangeably herein and refer to the amino acid residues of an antibody which are responsible for antigen-binding. The hypervariable region comprises amino acid residues from the complementarity determining regions or CDRs. “Framework” or “FR” regions are those variable domain regions other than the hypervariable region residues as herein defined. Therefore, the light and heavy chains of an antibody comprise from N- to C-terminus the domains FR1, CDR1, FR2, CDR2, FR3, CDR3, and FR4. Especially, CDR3 of the heavy chain is the region which contributes most to antigen binding. CDR and FR regions are determined according to the standard definition of Kabat et al., Sequences of Proteins of Immunological Interest, 5th Ed. Public Health Service, National Institutes of Health, Bethesda, Md. (1991)) and/or those residues from a “hypervariable loop.”

The terms “binding to IGF-IR” or “specific binding to IGF-IR” are used interchangeably herein and mean the binding of the antibody to IGF-IR in an in vitro assay, preferably in a binding assay in which the antibody is bound to a surface and binding of IGF-IR is measured by Surface Plasmon Resonance (SPR). Binding means a binding affinity (K_{D}) of 10⁻⁸ M or less, preferably 10⁻¹³ to 10⁻⁹ M. Binding to IGF-IR can be investigated by a BIAcore assay (Pharmacia Biosensor AB, Uppsala, Sweden). The affinity of the binding is defined by the terms ka (rate constant for the association of the antibody from the antibody/antigen complex), kd (dissociation constant), and K_{D} (kd/ka). The antibodies used in the methods disclose herein typically show a K_{D} of about 10⁻⁹ M or less.

The antibodies, or antigen binding fragments thereof, used in the methods disclosed herein inhibit the binding of IGF-I and IGF-II to IGF-IR. The inhibition is measured as IC₅₀ in an assay for binding of IGF-I/IGF-II to IGF-IR on cells. Such an assay is known to one of skill in the art and is described, for example, U.S. Pat. No. 7,579,157, which is incorporated herein in its entirety. The IC₅₀ values of the antibodies used in the methods disclosed herein for the binding of IGF-I and IGF-II to IGF-IR are no more than 2 nM. IC₅₀ values are measured as average or median values of at least three independent measurements. Single IC₅₀ values may be out of the scope.

The term “inhibiting the binding of IGF-I and IGF-II to IGF-IR” as used herein may refer to, e.g., inhibiting the binding of I¹²⁵-labeled IGF-I or IGF-II to IGF-IR presented on the surface of cells in an in vitro assay. Inhibiting means an IC₅₀ value of 10 nM, 5 nM, 2 nM, or lower.

The phrase “therapeutically effective” is intended to qualify the amount of active ingredients used in the treatment of a disease or disorder or on the effecting of a clinical endpoint.

The term “therapeutically acceptable” refers to those compounds (or salts, prodrugs, tautomers, zwitterionic forms, etc.) which are suitable for use in contact with the tissues of patients without undue toxicity, irritation, and allergic response, are commensurate with a reasonable benefit/risk ratio, and are effective for their intended use.

As used herein, reference to “treatment” of a subject or patient is intended to include prevention, prophylaxis, attenuation, amelioration, and therapy. Treatment may also include prevention of disease. Prevention of a disease may involve complete protection from disease, for example as in the case of prevention of infection with a pathogen, or may involve prevention of disease progression. For example, prevention of a disease may not mean complete foreclosure of any effect related to the diseases at any level, but instead may mean prevention of the symptoms of a disease to a clinically significant or detectable level. Prevention of diseases may also mean prevention of progression of a disease to a later stage of the disease.

The terms “subject” and “patient” are used interchangeably herein to mean all mammals including humans. Examples of subjects include, but are not limited to, humans, monkeys, dogs, cats, horses, cows, goats, sheep, pigs, and rabbits. In one embodiment, the subject or patient is a human.

The terms “affected with a disease or disorder,” “afflicted with a disease or disorder,” or “having a disease or disorder” are used interchangeably herein and refer to a subject or patient with any disease, disorder, syndrome, or condition. No increased or decreased level of severity of the disorder is implied by the use of one the terms as compared to the other.

The term “disease” as used herein is intended to be generally synonymous, and is used interchangeably with, the terms “disorder,” “syndrome,” and “condition” (as in medical condition), in that all reflect an abnormal condition of the human or animal body or of one of its parts that impairs normal functioning, is typically manifested by distinguishing signs and symptoms, and causes the human or animal to have a reduced duration or quality of life.

The term “combination therapy” means the administration of two or more therapeutic agents to treat a therapeutic condition or disorder described in the present disclosure. Such administration encompasses co-administration of these therapeutic agents in a substantially simultaneous manner, such as in a single capsule having a fixed ratio of active ingredients or in multiple, separate capsules for each active ingredient. In addition, such administration also encompasses use of each type of therapeutic agent in a sequential manner. In either case, the treatment regimen will provide beneficial effects of the drug combination in treating the conditions or disorders described herein.

When introducing elements of the present disclosure or the preferred embodiment(s) thereof, the articles “a,” “an,” “the” and “said” are intended to mean that there are one or more of the elements. The terms “comprising,” “including” and “having” are intended to be inclusive and mean that there may be additional elements other than the listed elements.

When ranges of values are disclosed, and the notation “from n1 . . . to n2” or “between n1 . . . and n2” is used, where n1 and n2 are the numbers, then unless otherwise specified, this notation is intended to include the numbers themselves and the range between them. This range may be integral or continuous between and including the end values. By way of example, the range “from 2 to 6 carbons” is intended to include two, three, four, five, and six carbons, since carbons come in integer units. Compare, by way of example, the range “from 1 to 3 μM (micromolar),” which is intended to include 1 μM, 3 μM, and everything in between to any number of significant figures (e.g., 1.255 μM, 2.1 μM, 2.9999 μM, etc.).

### Antibodies

The sequences of the heavy chains and light chains of examples of antibodies that may be used in the methods disclosed herein, each comprising three CDRs on the heavy chain and three CDRs on the light chain are provided below. The sequences of the CDRs, heavy chains, light chains as well as the sequences of the nucleic acid molecules encoding the CDRs, heavy chains and light chains of the antibodies are disclosed in the sequence listing. The CDRs of the antibody heavy chains are referred to as CDRH1 (or HCDR1), CDRH2 (or HCDR2) and CDRH3 (or HCDR3), respectively. Similarly, the CDRs of the antibody light chains are referred to as CDRL1 (or LCDR1), CDRL2 (or LCDR2) and CDRL3 (or LCDR3), respectively. Table 2 provides the SEQ ID numbers for the amino acid sequences of the six CDRs of the heavy and light chains, respectively, of the antibodies that may be used in the methods disclosed herein.

TABLE 2
SEQ ID Numbers for CDR Polypeptides
of Antibodies Disclosed Herein.

| | SEQ ID NOs. for CDR Polypeptides | | | | | |
|---|---|---|---|---|---|---|
| | CDRH1 | CDRH2 | CDRH3 | CDRL1 | CDRL2 | CDRL3 |
| Antibody 1 | 84 | 85 | 86 | 87 | 88 | 89 |
| Antibody 2 | 84 | 92 | 86 | 87 | 93 | 89 |

In one embodiment, an antibody or antibody fragment useful in the methods disclosed herein comprises at least one CDR with a sequence that has at least 95% sequence identity to any one of SEQ ID NOs: 84-89, 92, or 93 and specifically inhibits (or blocks) Insulin Like Growth Factor-I Receptor (IGF-IR).

In another embodiment, the antibody or antigen binding fragment that can be used in the methods comprising a heavy chain comprises one or more (i.e. one, two or all three) heavy chain CDRs from antibody 1 or antibody 2 and specifically inhibits or blocks IGF-IR.

In yet another embodiment, the antibody or antigen binding fragment useful in the methods disclosed herein comprises a heavy chain CDR1 with the amino acid sequence of SEQ ID NO: 84; a heavy chain CDR2 with the amino acid sequence of SEQ ID NO: 85, or SEQ ID NO: 92; and a heavy chain CDR3 with the amino acid sequence of SEQ ID NO: 86. In certain embodiments, the antibody or antibody fragment comprises a heavy chain comprising the amino acid sequence of (i) SEQ ID NO: 84 for CDRH1, SEQ ID NO: 85 for CDRH2 and SEQ ID NO: 86 for CDRH3; or (ii) SEQ ID NO: 84 for CDRH1, SEQ ID NO: 92 for CDRH2, and SEQ ID NO: 86 for CDRH3 and specifically inhibits IGF-IR.

In another embodiment, the antibody or antigen binding fragment that can be used in the methods disclosed herein comprising a light chain comprising one or more (i.e. one, two or all three) light chain CDRs from antibody 1 or antibody 2 and specifically inhibits IGF-IR.

In yet another embodiment, an antibody or antibody fragment useful in the methods disclosed herein comprises a light chain CDR1 with the amino acid sequence of SEQ ID NO: 87; a light chain CDR2 with the amino acid sequence of SEQ ID NO: 88, or SEQ ID NO: 93; and a light chain CDR3 with the amino acid sequence of SEQ ID NO: 89. In certain embodiments, the antibody or antibody fragment comprises a light chain comprising the amino acid sequence of (i) SEQ ID NO: 87 for CDRL1, SEQ ID NO: 88 for CDRL2, and SEQ ID NO: 89 for CDRL3; or (ii) SEQ ID NO: 87 for CDRL1, SEQ ID NO: 93 for CDRL2, and SEQ ID NO: 89 for CDRL3.

In one embodiment, the antibody, or antigen binding fragment thereof, comprises all of the CDRs of antibody 1 as listed in Table 2, and specifically inhibits (or blocks) Insulin Like Growth Factor-I Receptor (IGF-IR). In another embodiment, an antibody, or antigen binding fragment thereof, comprises all of the CDRs of antibody 2 as listed in Table 2, and specifically inhibits (or blocks) IGF-IR.

The SEQ ID numbers for the amino acid sequence for the heavy chain variable region (VH) and the light chain variable region (VL) of antibodies useful in the methods disclosed herein are listed in Table 3.

TABLE 3
SEQ ID Numbers for V_{H} and V_{L} amino acid
for Antibodies Disclosed Herein.

| | V_{H} amino acid | V_{L} amino acid |
|---|---|---|
| Antibody 1 | 90 | 91 |
| Antibody 2 | 94 | 95 |

In one embodiment, the antibody or antigen binding fragment that can be used in the methods disclosed herein comprises a heavy chain variable region having an amino acid sequence that is about 70%, 75%, 80%, 85%, 90%, 95%, 97%, 98%, 99% or 100% identical to the sequence recited in SEQ ID NOs: 90 or 94 wherein the antibody specifically inhibits IGF-IR.

In another embodiment, the antibody or antigen binding fragment that can be used in the methods disclosed herein specifically inhibits IGF-IR and comprises a heavy chain variable region comprising the amino acid sequence of SEQ ID NO: 90 and a light chain variable region comprising the amino acid sequence of SEQ ID NO: 91; or a heavy chain variable region comprising the amino acid sequence of SEQ ID NO: 94 and a light chain variable region comprising the amino acid sequence of SEQ ID NO: 95.

Examples of antibodies useful in the methods disclosed herein include, but are not limited to, antibody 1, and antibody 2. In some embodiments, antibody 1 is teprotumumab.

Variant antibodies are also included within the scope of the disclosure. Thus, variants of the sequences recited in the application are also included within the scope of the disclosure. Such variants include natural variants generated by somatic mutation in vivo during the immune response or in vitro upon culture of immortalized B cell clones. Alternatively, variants may arise due to the degeneracy of the genetic code or may be produced due to errors in transcription or translation.

Further variants of the antibody sequences having improved affinity and/or potency may be obtained using methods known in the art and are included within the scope of the disclosure. For example, amino acid substitutions may be used to obtain antibodies with further improved affinity. Alternatively, codon optimization of the nucleotide sequence may be used to improve the efficiency of translation in expression systems for the production of the antibody. Further, polynucleotides comprising a sequence optimized for antibody specificity or neutralizing activity by the application of a directed evolution method to any of the nucleic acid sequences disclosed herein are also within the scope of the disclosure.

In one embodiment variant antibody sequences may share 70% or more (i.e. 75%, 80%, 85%, 90%, 95%, 97%, 98%, 99% or more) amino acid sequence identity with the sequences recited in the application. In some embodiments such sequence identity is calculated with regard to the full length of the reference sequence (i.e. the sequence recited in the application). In some further embodiments, percentage identity, as referred to herein, is as determined using BLAST version 2.1.3 using the default parameters specified by the NCBI (the National Center for Biotechnology Information.

Antibodies used with the methods disclosed herein can be coupled to a drug for delivery to a treatment site or coupled to a detectable label to facilitate imaging of a site comprising cells of interest. Methods for coupling antibodies to drugs and detectable labels are well known in the art, as are methods for imaging using detectable labels. Labeled antibodies may be employed in a wide variety of assays, employing a wide variety of labels. Detection of the formation of an antibody-antigen complex between an antibody and an epitope of interest can be facilitated by attaching a detectable substance to the antibody and detecting the antibody-antigen complex by suitable detection means known to one of skill in the art.

Antibodies, or antigen binding fragments thereof, used with the methods disclosed herein can be of any isotype (e.g., IgA, IgG, IgM; i.e., an α, γ or β heavy chain). In one embodiment the antibody is IgG. Within the IgG isotype, antibodies may be IgG1, IgG2, IgG3 or IgG4 subclass. The antibodies may have a κ or a λ light chain.

The antibodies, or an antigen binding fragments thereof, used with the methods disclosed herein can be administered by any route known to one of skill in the art. Non-exhaustive examples of routes that can be used are provided below.

### Antibody Fc Variants and Half-Life

In immunoglobulins, such as IgG, a site in the Fc region of the heavy chain mediates interaction with the neonatal receptor (FcRn). Binding to FcRn recycles endocytosed antibody from the endosome back to the bloodstream and plays a key role in antibody transport. This process, coupled with preclusion of kidney filtration due to the large size of the full-length molecule, results in favorable antibody serum half-lives ranging from one to three weeks in vivo. Thus, the fidelity of this region on Fc is important for the clinical properties of antibodies.

Other properties of the antibody may determine its clearance rate (e.g. stability and half-life) in vivo. In addition to antibody binding to the FcRn receptor, other factors that contribute to clearance and half-life are serum aggregation, enzymatic degradation in the serum, inherent immunogenicity of the antibody leading to clearing by the immune system, antigen-mediated uptake, FcR (non-FcRn) mediated uptake and non-serum distribution (e.g. in different tissue compartments).

Accordingly, one means by which the pharmacokinetics (PK) and pharmacodynamics (PD) of a therapeutic antibody can by changed is by increasing the serum half-life of the antibody by altering the heavy constant domains within the Fc. In addition, due to the methodologies outlined herein, the possibility of immunogenicity resulting from the FcRn variants is significantly reduced by importing variants from different IgG isotypes such that serum half-life is increased without introducing significant immunogenicity.

The substitutions in the Fc domains are chosen such that the resultant proteins show improved serum half-life in vivo as compared to the wild type protein. In order to increase the retention of the Fc proteins in vivo, the increase in binding affinity must be at around pH 6 while maintaining lower affinity at around pH 7.4. Without being limited to theory, Fc regions are believed to have longer half-lives in vivo because binding to FcRn at pH 6 in an endosome sequesters the Fc. The endosomal compartment then recycles the Fc to the cell surface. Once the compartment opens to the extracellular space, the higher pH (˜7.4) induces the release of Fc back into the blood. The increased affinity of Fc for FcRn at pH 7.4 is thought to forbid the release of the Fc back into the blood. As a result, Fc mutations that increase Fc's half-life in vivo generally increase FcRn binding at the lower pH while still allowing release of Fc at higher pH. The amino acid histidine changes its charge state in the pH range of 6.0 to 7.4. Therefore, it is not surprising to find histidine residues at important positions in the Fc/FcRn complex.

In some embodiments, the increase in FcRn binding over wild type specifically at lower pH (˜6.0) facilitates Fc/FcRn binding in the endosome. In some embodiments, Fc variants with altered FcRn binding can have altered binding to another class of Fc receptors, the FcγRs (FcgammaRs) as differential binding to FcγR5, particularly increased binding to FcγRIIIb and decreased binding to FcγRIIb, has been shown to result in increased efficacy.

In some embodiments, importation of substitutions at particular positions from one IgG isotype into another can be achieved, thus reducing or eliminating the possibility of unwanted immunogenicity being introduced into the variants. That is, IgG1 is a common isotype for therapeutic antibodies for a variety of reasons, including high effector function. IgG2 residues at particular positions can be introduced into the IgG1 backbone to result in a protein that exhibits longer serum half-life.

In some embodiments, non-isotypic amino acid changes are made, to improve binding to FcRn and/or to increase in vivo serum half-life, and/or to allow accommodations in structure for stability, etc.

As will be appreciated by those in the art and described below, a number of factors contribute to the in vivo clearance, and thus the half-life, of antibodies in serum. One factor involves the antigen to which the antibody binds; that is, antibodies with identical constant regions but different variable regions (e.g., Fv domains), may have different half-lives due to differential ligand binding effects. However, the present disclosure demonstrates that while the absolute half-life of two different antibodies may differ due to these antigen specificity effects, the FcRn variants described herein, can transfer to different ligands to give the same trends of increasing half-life. That is, in general, the relative “order” of the FcRn binding/half-life increases will track to antibodies with the same variants of antibodies with different Fvs as is discussed herein.

Fc variants within a therapeutic antibody are made by introducing amino acid mutations into the parent molecule. “Mutations” in this context are usually amino acid substitutions, although as shown herein, deletions and insertions of amino acids can also be done and thus are defined as mutations.

The Fc variant antibodies of the disclosure show increased binding to FcRn and/or increased in vivo serum half-life. By “FcRn” or “neonatal Fc Receptor” as used herein is meant a protein that binds the IgG antibody Fc region and is encoded at least in part by an FcRn gene. The FcRn may be from any organism, including but not limited to humans, mice, rats, rabbits, and monkeys. As is known in the art, the functional FcRn protein comprises two polypeptides, often referred to as the heavy chain and light chain. The light chain is beta-2-microglobulin and the heavy chain is encoded by the FcRn gene. Unless otherwise noted herein, FcRn or an FcRn protein refers to the complex of FcRn heavy chain with beta-2-microglobulin. In some cases, the FcRn variants bind to the human FcRn receptor, or it may be desirable to design variants that bind to rodent or primate receptors in addition, to facilitate clinical trials.

In some embodiments, the present disclosure provides compositions and methods of administering an antibody to a subject, where the antibody comprises a variant Fc region as compared to a parent Fc region, wherein the variant Fc region comprises a first mutation that is a leucine at position 428 and a second mutation that is a serine at position 434, where the antibody has increased serum half-life as compared to an antibody comprising the parent Fc region, and wherein numbering is according to the EU index. In some embodiments, the antibody disclosed herein comprises a variant Fc region comprising mutations that substitute a methionine at position 428 with a leucine (Met428Leu) and substitutes an asparagine at position 434 with a serine (Asn434Ser). Numbering is EU as in Kabat, and it is understood that the substitution is non-native to the starting molecule. As has been shown previously, these FcRn substitutions work in IgG1, IgG2 and IgG1/G2 hybrid backbones, and are specifically included for IgG3 and IgG4 backbones and derivatives of any IgG isoform as well.

The present disclosure includes variants of Fc domains, including those found in antibodies, Fc fusions, and immuno-adhesions, which have an increased binding to the FcRn receptor. As noted herein, binding to FcRn results in longer serum retention in vivo. A variety of such substitutions—including those related to M428L/N434S—are known and described in U.S. Pat. Nos. 7,317,091; 8,084,582; and 8,101,720; 8,188,231; 8,367,805; and 8,546,543, each of which is incorporated herein by reference in their entirety.

### Dosing and Administration

The compound, antibody, or an antigen binding fragment thereof, can be administered in a single dose or in multiple doses. In some embodiments, the therapeutic antibody is administered to the subject in a single dose. In some embodiments, the therapeutic antibody is administered to the subject in multiple doses, spread out over the course of a few days, weeks, or months. In some embodiments the antibody, or an antigen binding fragment thereof, is administered every week or every 2 weeks or every 3 weeks or every 4 weeks or every 5 weeks or every 6 weeks or every 7 weeks or every 8 weeks or every month or every 2 months or every 3 months.

In some embodiments the antibody, or an antigen binding fragment thereof, is administered in multiple doses and the dosage is the same each time. In some embodiments the antibody, or an antigen binding fragment thereof, is administered in multiple doses and the dosage at the time of first administration is different (could be higher or lower) from those at subsequent times. In some embodiments the antibody, or an antigen binding fragment thereof, is administered in multiple doses and the dosage is adjusted at each administration based on the subject's response to the therapy.

The dosage may further vary between patients, based on different factors such as the age, gender, race, and body weight of each patient. In some embodiments, the dosage varies by body weight of the patient. The dosage could range from about 1 mg of the antibody, or an antigen binding fragment thereof, per kilogram of body weight to about 100 mg of the antibody, or an antigen binding fragment thereof, per kilogram of body weight. The dosage, could for example, be 1 mg, 2 mg, 3 mg, 5 mg, 7 mg, 10 mg, 12 mg, 15 mg, 17 mg, 20 mg, 22 mg, 25 mg, 30 mg, 35 mg, 40 mg, 45 mg, 50 mg, 55 mg, 60 mg, 65 mg, 70 mg, 75 mg, 80 mg, 85 mg, 90 mg, 95 mg or 100 mg, of the antibody, or an antigen binding fragment thereof, per kilogram of body weight.

In some embodiments, the dose is about 0.3 mg/kg to about 10 mg/kg of the antibody, or an antigen binding fragment thereof. In some embodiments, the dosage is about 0.3 mg/kg to about 5 mg/kg of the antibody, or an antigen binding fragment thereof. In some embodiments, the dosage is about 0.3 mg/kg to about 1 mg/kg of the antibody, or an antigen binding fragment thereof. The dosage, could for example, be about 0.3 mg/kg, about 0.4 mg/kg, about 0.5 mg/kg, about 0.6 mg/kg, about 0.7 mg/kg, about 0.8 mg/kg, about 0.9 mg/kg, about 1 mg/kg, about 1.1 mg/kg, about 1.2 mg/kg, about 1.3 mg/kg, about 1.4 mg/kg, about 1.5 mg/kg, 1.6 mg/kg, 1.7 mg/kg, 1.8 mg/kg, 1.9 mg/kg, about 2 mg/kg, about 2.5 mg/kg, about 3 mg/kg, about 3.5 mg/kg, about 4 mg/kg, about 4.5 mg/kg, about 5 mg/kg, about 5.5 mg/kg, about 6 mg/kg, about 6.5 mg/kg, about 7 mg/kg, about 7.5 mg/kg, about 8 mg/kg, about 8.5 mg/kg, about 9 mg/kg, about 9.5 mg/kg, or about 10 mg/kg, or any number of tenths of a mg/kg in between the foregoing, of the antibody, or an antigen binding fragment thereof. In some embodiments, the dose is administered every week.

In some embodiments, the dose is about 0.6 mg/kg to about 20 mg/kg of the antibody, or an antigen binding fragment thereof. In some embodiments, the dosage is about 0.6 mg/kg to about 5 mg/kg of the antibody, or an antigen binding fragment thereof. In some embodiments, the dosage is about 0.6 mg/kg to about 10 mg/kg of the antibody, or an antigen binding fragment thereof. The dosage, could for example, be about 0.6 mg/kg, about 0.7 mg/kg, about 0.8 mg/kg, about 0.9 mg/kg, about 1 mg/kg, about 1.1 mg/kg, about 1.2 mg/kg, about 1.3 mg/kg, about 1.4 mg/kg, about 1.5 mg/kg, 1.6 mg/kg, 1.7 mg/kg, 1.8 mg/kg, 1.9 mg/kg, about 2 mg/kg, about 2.5 mg/kg, about 3 mg/kg, about 3.5 mg/kg, about 4 mg/kg, about 4.5 mg/kg, about 5 mg/kg, about 5.5 mg/kg, about 6 mg/kg, about 6.5 mg/kg, about 7 mg/kg, about 7.5 mg/kg, about 8 mg/kg, about 8.5 mg/kg, about 9 mg/kg, about 9.5 mg/kg, about 10 mg/kg, about 11 mg/kg, about 12 mg/kg, about 13 mg/kg, about 14 mg/kg, about 15 mg/kg, about 16 mg/kg, about 17 mg/kg, about 18 mg/kg, about 19 mg/kg, or about 20 mg/kg, or any number of tenths of a mg/kg in between the foregoing, of the antibody, or an antigen binding fragment thereof. In some embodiments, the dose is administered every two weeks.

In some embodiments, the dose is about 1 mg/kg to about 30 mg/kg of the antibody, or an antigen binding fragment thereof. In some embodiments, the dose is about 5 mg/kg to about 30 mg/kg of the antibody, or an antigen binding fragment thereof. In some embodiments, the dose is about 10 mg/kg to about 30 mg/kg of the antibody, or an antigen binding fragment thereof. The dosage, could for example, be about 1 mg/kg, about 2 mg/kg, about 3 mg/kg, about 5 mg/kg, about 7 mg/kg, about 10 mg/kg, about 12 mg/kg, about 15 mg/kg, about 17 mg/kg, about 20 mg/kg, about 22 mg/kg, about 25 mg/kg, or about 30 mg/kg, or any integer and/or number of tenths of a mg/kg in between the foregoing, of the antibody, or an antigen binding fragment thereof. In some embodiments, the dose is administered every three weeks.

In some embodiments, the dose is about 1.2 mg/kg to about 40 mg/kg of the antibody, or an antigen binding fragment thereof. In some embodiments, the dose is about 5 mg/kg to about 40 mg/kg of the antibody, or an antigen binding fragment thereof. In some embodiments, the dose is about 10 mg/kg to about 40 mg/kg of the antibody, or an antigen binding fragment thereof. In some embodiments, the dose is about 20 mg/kg to about 40 mg/kg of the antibody, or an antigen binding fragment thereof. In some embodiments, the dose is about 25 mg/kg to about 40 mg/kg of the antibody, or an antigen binding fragment thereof. The dosage, could for example, be about 1 mg/kg, about 2 mg/kg, about 3 mg/kg, about 5 mg/kg, about 7 mg/kg, about 10 mg/kg, about 12 mg/kg, about 15 mg/kg, about 17 mg/kg, about 20 mg/kg, about 22 mg/kg, about 25 mg/kg, about 27 mg/kg, about 30 mg/kg, about 32 mg/kg, about 35 mg/kg, about 37 mg/kg, or about 40 mg/kg, or any integer and/or number of tenths of a mg/kg in between the foregoing, of the antibody, or an antigen binding fragment thereof. In some embodiments, the dose is administered every four weeks.

In some embodiments, the dosage is about 1 mg/kg to about 5 mg/kg of the antibody, or an antigen binding fragment thereof. In some embodiments, the dosage is about 5 mg/kg to about 10 mg/kg of the antibody, or an antigen binding fragment thereof. In some embodiments, the dosage is about 10 mg/kg to about 15 mg/kg of the antibody, or an antigen binding fragment thereof. In some embodiments, the dosage is about 15 mg/kg to about 20 mg/kg of the antibody, or an antigen binding fragment thereof.

In some embodiments, the dosage is about 1 mg/kg to about 5 mg/kg of the antibody, or an antigen binding fragment thereof. In some embodiments, the dosage is about 5 mg/kg to about 10 mg/kg of the antibody, or an antigen binding fragment thereof. In some embodiments, the dosage is about 10 mg/kg to about 15 mg/kg of the antibody, or an antigen binding fragment thereof. In some embodiments, the dosage is about 15 mg/kg to about 20 mg/kg of the antibody, or an antigen binding fragment thereof.

In some embodiments the antibody, or an antigen binding fragment thereof, is administered in multiple doses and the dosage at the time of first administration is different from those at subsequent times, the dosage at the time of first administration is about 1 mg/kg to about 5 mg/kg of the antibody, or an antigen binding fragment thereof; or about 5 mg/kg to about 10 mg/kg of the antibody, or an antigen binding fragment thereof; or about 10 mg/kg to about 15 mg/kg of the antibody, or an antigen binding fragment thereof; or about 15 mg/kg to about 20 mg/kg of the antibody, or an antigen binding fragment thereof; or about 20 mg/kg to about 25 mg/kg of the antibody, or an antigen binding fragment thereof. The subsequent dose(s) could be higher or lower than the first dose. In some embodiments, the subsequent dose is about 1 mg/kg to about 5 mg/kg of the antibody, or an antigen binding fragment thereof; or about 5 mg/kg to about 10 mg/kg of the antibody, or an antigen binding fragment thereof; or about 10 mg/kg to about 15 mg/kg of the antibody, or an antigen binding fragment thereof; or about 15 mg/kg to about 20 mg/kg of the antibody, or an antigen binding fragment thereof; or about 20 mg/kg to about 25 mg/kg of the antibody, or an antigen binding fragment thereof.

Small molecule compounds may be administered orally, via injection, etc. at a dose of from 0.01 to 500 mg/kg per day and/or from 0.1 mg to 5 g per day. The dose range for adult humans is generally from 5 mg to 2 g/day. Tablets or other forms of presentation provided in discrete units may conveniently contain an amount of one or more compounds which is effective at such dosage or as a multiple of the same, for instance, units containing 5 mg to 500 mg, for example around 10 mg to 200 mg.

The amount of active ingredient that may be combined with the carrier materials to produce a single dosage form will vary depending upon the host treated and the particular mode of administration. The precise amount of compound administered to a patient will be the responsibility of the attendant physician. The specific dose level for any particular patient will depend upon a variety of factors including the activity of the specific compound employed, the age, body weight, general health, sex, diets, time of administration, route of administration, rate of excretion, drug combination, the precise disorder being treated, and the severity of the indication or condition being treated. Also, the route of administration may vary depending on the condition and its severity.

Additional dosage ranges are provided throughout this disclosure.

The duration of the treatment depends on the subject's response to the therapy and can range from about one month or 4 weeks to about 2 years or 100 weeks. In some embodiments, the treatment may be provided over a total duration of about 1 month, 2 months, 3 months, 4 months, 5 months, 6 months, 7 months, 8 months, 9 months, 10 months, 11 months, 1 year, 14 months, 16 months, 18 months, 20 months, 22 months, or 2 years. In some embodiments, the treatment may be provided over a total duration of 4, 6, 8, 10, 12, 14, 16, 20, 22, 24, 26, 28, 30, 32, 34, 36, 38, 40, 42, 44, 46, 48, 50, 52 weeks, or extended to 56, 64, 72, 80, 88, 96 or 104 weeks.

In some embodiments, the antibody, or an antigen binding fragment thereof, is administered for a duration of 24 weeks at intervals of 3 weeks starting with an initial dose of 10 mg per kilogram of body weight, followed by 20 mg per kilogram for seven additional treatments. In some embodiments, the slam molecule compound is administered daily (QD), twice daily, (BID) or thrice daily (TID) for an appropriate duration, e.g., 24 weeks.

The compound, salt, or polymorph thereof, antibody, or antigen binding fragment thereof, may be administered by any suitable route including, but not limited to, oral, intravenous, intraorbitally, periorbitally, intramuscular, intra-arterial, intramedullary, intraperitoneal, intrathecal, intraventricular, transdermal, transcutaneous, topical, subcutaneous, intranasal, enteral, sublingual, intravaginal or rectal routes. Hyposprays may also be used to administer the pharmaceutical compositions disclosed herein. Typically, the therapeutic antibody may be prepared as a freeze-dried (lyophilized) powder or as an injectable, either as a liquid solution or suspension. Solid forms suitable for solution in, or suspension in, liquid vehicles prior to injection may also be used.

### Pharmaceutical Compositions

The pharmaceutical compositions used in the methods disclosed herein comprise one or more of: the antibodies or antibody fragments described above and a pharmaceutically acceptable carrier or excipient. Although the carrier or excipient may facilitate administration, it should not itself induce the production of antibodies harmful to the subject or individual receiving the composition; nor should it be toxic. Suitable carriers may be large, slowly metabolized macromolecules such as proteins, polypeptides, liposomes, polysaccharides, polylactic acids, polyglycolic acids, polymeric amino acids, amino acid copolymers and inactive virus particles, and are known to one of skill in the art.

The antibodies, or an antigen binding fragments thereof, or pharmaceutical compositions used with the methods disclosed herein may be administered by any number of routes including, but not limited to, oral, intravenous, intramuscular, intra-arterial, intramedullary, intraperitoneal, intrathecal, intraventricular, transdermal, transcutaneous, topical, subcutaneous, intranasal, enteral, sublingual, intravaginal or rectal routes. Hyposprays may also be used to administer the pharmaceutical compositions disclosed herein. Typically, the therapeutic compositions may be prepared as injectables, either as liquid solutions or suspensions. Solid forms suitable for solution in, or suspension in, liquid vehicles prior to injection may also be prepared.

In one embodiment, the antibody, or an antigen binding fragment thereof, or pharmaceutical composition is administered intravenously. In another embodiment, the antibody, or an antigen binding fragment thereof, or pharmaceutical composition is administered by intravenous infusion.

Direct delivery of the compositions will generally be accomplished by injection, subcutaneously, intraperitoneally, intravenously, or intramuscularly, or delivered to the interstitial space of a tissue. The compositions can also be administered into a lesion. Dosage treatment may be a single dose schedule or a multiple dose schedule. Known antibody-based pharmaceuticals provide guidance relating to frequency of administration e.g., whether a pharmaceutical should be delivered daily, weekly, monthly, etc. Frequency and dosage may also depend on the severity of symptoms.

It will be appreciated that the active ingredient in the composition will be an antibody molecule, an antibody fragment or variants and derivatives thereof. As such, it will be susceptible to degradation in the gastrointestinal tract. Thus, if the composition is to be administered by a route using the gastrointestinal tract, the composition will need to contain agents which protect the antibody from degradation, but which release the antibody once it has been absorbed from the gastrointestinal tract.

For larger molecular weight moieties such as mAbs (˜150 kDa), the SC capillaries have low passive permeability; absorption of mAbs into systemic circulation occurs via lymphatic uptake from the interstitial space, as well as via active transport by the neonatal Fc receptor (FcRn) across the capillary endothelia. The extracellular matrix of the subcutaneous tissue also limits the injection of larger volumes (>1-2 mL) SC generally; coformulation with a recombinant hyaluronidase or soluble fragment thereof such as rHuPH20 can permit higher bioavailability. Additionally, physiochemical properties of mAbs, including charge, hydrophobicity, and stability, affect the rate and extent of their SC absorption; for example, the combination of high positive charge and hydrophobic interaction can reduce the rate absorption.

In some embodiments, pharmaceutical compositions suitable for use in the methods disclosed herein are formulated for subcutaneous administration. Examples of formulations suitable for subcutaneous administration include, but are not limited to, solutions, suspensions, emulsions, and dry products that can be dissolved or suspended in a pharmaceutically acceptable carrier for injection. Antibodies have been, and may be, formulated for subcutaneous administration using methods known in the art.

Pharmaceutical compositions suitable for use in the methods disclose herein comprise one or more pharmaceutically acceptable carriers, such as those widely employed in the art of drug manufacturing, and particularly antibody drug manufacturing. Pharmaceutically acceptable carriers in particular are non-toxic and should not interfere with the efficacy of the active ingredient. The carrier may be a diluent, adjuvant, excipient, or vehicle with which the antibodies are administered. Such vehicles may be liquids, such as aqueous fluids, oils, and emulsions. For example, 0.4% saline and 0.3% glycine may be used. The solutions are sterile and generally free of particulate matter. They may be sterilized by conventional, well-known sterilization techniques (e.g., filtration). The compositions may contain pharmaceutically acceptable auxiliary substances as required to approximate physiological conditions such as pH adjusting and buffering agents, stabilizing, thickening, lubricating, and coloring agents, etc. The concentration of the antibodies in such pharmaceutical formulation may vary and will be selected primarily based on required dose, fluid volumes, viscosities, etc., according to the particular mode of administration selected, and other concerns, such as protein aggregation.

Examples of pharmaceutically acceptable carriers are solvents, dispersion media, coatings, antibacterial and antifungal agents, isotonic and absorption delaying agents, and the like that are physiologically compatible, such as salts, buffers, antioxidants, saccharides, aqueous or non-aqueous carriers, preservatives, wetting agents, surfactants or emulsifying agents, permeation enhancers, or combinations thereof.

Examples of buffers that may be used are acetic acid, citric acid, formic acid, succinic acid, phosphoric acid, carbonic acid, malic acid, aspartic acid, histidine, boric acid, Tris buffers, HEPPSO and HEPES.

Examples of antioxidants that may be used are ascorbic acid, methionine, cysteine hydrochloride, sodium bisulfate, sodium metabisulfite, sodium sulfite, lecithin, citric acid, ethylenediamine tetraacetic acid (EDTA), sorbitol and tartaric acid.

Examples of amino acids that may be used are histidine, isoleucine, methionine, glycine, arginine, lysine, L-leucine, tri-leucine, alanine, glutamic acid, L-threonine, and 2-phenylamine.

Examples of surfactants that may be used are polysorbates (e.g., polysorbate-20 or polysorbate-80); polyoxamers (e.g, poloxamer 188); Triton; sodium octyl glycoside; lauryl-, myristyl-, linoleyl-, or stearyl-sulfobetaine; lauryl-, myristyl-, linoleyl- or stearyl-sarcosine; linoleyl-, myristyl-, or cetyl-betaine; lauroamidopropyl-, cocamidopropyl-, linoleamidopropyl-, myristamidopropyl-, palmidopropyl-, or isostearamidopropyl-betaine (e.g, lauroamidopropyl); myristamidopropyl-, palmidopropyl-, or isostearamidopropyl-dimethylamine; sodium methyl cocoyl-, or disodium methyl oleyl-taurate; and the MONAQUA™ series (Mona Industries, Inc., Paterson, N. J.), polyethyl glycol, polypropyl glycol, and copolymers of ethylene and propylene glycol (e.g PLURONICS™, PF68, etc.).

Examples of preservatives that may be used are phenol, m-cresol, p-cresol, o-cresol, chlorocresol, benzyl alcohol, phenylmercuric nitrite, phenoxyethanol, formaldehyde, chlorobutanol, magnesium chloride, alkylparaben (methyl, ethyl, propyl, butyl and the like), benzalkonium chloride, benzethonium chloride, sodium dehydroacetate and thimerosal, or mixtures thereof.

Examples of saccharides that may be used are monosaccharides, disaccharides, trisaccharides, polysaccharides, sugar alcohols, reducing sugars, nonreducing sugars such as glucose, sucrose, trehalose, lactose, fructose, maltose, dextran, glycerin, dextran, erythritol, glycerol, arabitol, sylitol, sorbitol, mannitol, mellibiose, melezitose, raffmose, mannotriose, stachyose, maltose, lactulose, maltulose, glucitol, maltitol, lactitol or iso-maltulose.

Examples of permeation enhancers that may be used include recombinant hyaluronidase or soluble fragment thereof such as rHuPH20 (Halozyme). Liquid formulations for subcutaneous administration may comprise rHuPH20 or another soluble human hyaluronidase enzyme. rHuPH20 may be present in an amount sufficient to result in an increase in the dispersion of the antibodies contained in the same liquid formulation during subcutaneous administration.

The amounts of pharmaceutically acceptable carrier(s) in the pharmaceutical compositions may be determined experimentally based on the activities of the carrier(s) and the desired characteristics of the formulation, such as stability, bioavailability, and/or minimal oxidation.

The methods of the present disclosure can use an antibody, or an antigen binding fragment thereof, as described above, alone or in combination with other pharmaceutically active compounds, to treat conditions such as those disclosed hereinabove. The additional pharmaceutically active compound(s) can be administered simultaneously (either in the same dosage form or in separate dosage forms) or sequentially. Accordingly, in one embodiment, the present disclosure comprises methods for treating a condition by administering to the subject a therapeutically-effective amount of an antibody, or an antigen binding fragment thereof, of the present disclosure and one or more additional pharmaceutically active compounds.

In some embodiments, the antibody, or an antigen binding fragment thereof, of the present disclosure is used in combination with existing therapies, including, but not limited to, corticosteroids; rituximab and other anti-CD20 antibodies; tocilizumab and other anti-IL-6 antibodies; or selenium, infliximab and other anti-TNF-α antibodies. In some embodiments, the antibody, or an antigen binding fragment thereof, of the present disclosure is used in combination with TSHR inhibitors.

### Salts, Polymorphs, and Solvates

The small molecule compounds disclosed herein can exist as therapeutically acceptable salts. The present invention includes compounds in the form of salts, including acid addition salts. Suitable salts include those formed with both organic and inorganic acids. Such acid addition salts will normally be pharmaceutically acceptable. However, salts of non-pharmaceutically acceptable salts may be of utility in the preparation and purification of the compound in question. Basic addition salts may also be formed and be pharmaceutically acceptable.

The term “therapeutically acceptable salt,” as used herein, represents salts or zwitterionic forms of the compounds disclosed herein which are water or oil-soluble or dispersible and therapeutically acceptable as defined herein. The salts can be prepared during the final isolation and purification of the compounds or separately by reacting the appropriate compound in the form of the free base with a suitable acid. Representative acid addition salts include acetate, adipate, alginate, L-ascorbate, aspartate, benzoate, benzenesulfonate (besylate), bisulfate, butyrate, camphorate, camphorsulfonate, citrate, digluconate, formate, fumarate, gentisate, glutarate, glycerophosphate, glycolate, hemisulfate, heptanoate, hexanoate, hippurate, hydrochloride, hydrobromide, hydroiodide, 2-hydroxyethansulfonate (isethionate), lactate, maleate, malonate, DL-mandelate, mesitylenesulfonate, methanesulfonate, naphthylenesulfonate, nicotinate, 2-naphthalenesulfonate, oxalate, pamoate, pectinate, persulfate, 3-phenylproprionate, phosphonate, picrate, pivalate, propionate, pyroglutamate, succinate, sulfonate, tartrate, L-tartrate, trichloroacetate, trifluoroacetate, phosphate, glutamate, bicarbonate, para-toluenesulfonate (p-tosylate), and undecanoate. Also, basic groups in the compounds disclosed herein can be quaternized with methyl, ethyl, propyl, and butyl chlorides, bromides, and iodides; dimethyl, diethyl, dibutyl, and diamyl sulfates; decyl, lauryl, myristyl, and steryl chlorides, bromides, and iodides; and benzyl and phenethyl bromides. Examples of acids which can be employed to form therapeutically acceptable addition salts include inorganic acids such as hydrochloric, hydrobromic, sulfuric, and phosphoric, and organic acids such as oxalic, maleic, succinic, and citric. Salts can also be formed by coordination of the compounds with an alkali metal or alkaline earth ion. Hence, the present invention contemplates sodium, potassium, magnesium, and calcium salts of the compounds disclosed herein, and the like.

Basic addition salts can be prepared during the final isolation and purification of the compounds by reacting a carboxy group with a suitable base such as the hydroxide, carbonate, or bicarbonate of a metal cation or with ammonia or an organic primary, secondary, or tertiary amine. The cations of therapeutically acceptable salts include lithium, sodium, potassium, calcium, magnesium, and aluminum, as well as nontoxic quaternary amine cations such as ammonium, tetramethylammonium, tetraethylammonium, methylamine, dimethylamine, trimethylamine, triethylamine, diethylamine, ethylamine, tributylamine, pyridine, N,N-dimethylaniline, N-methylpiperidine, N-methylmorpholine, dicyclohexylamine, procaine, dibenzylamine, N,N-dibenzylphenethylamine, 1-ephenamine, and N,N-dibenzylethylenediamine. Other representative organic amines useful for the formation of base addition salts include ethylenediamine, ethanolamine, diethanolamine, piperidine, and piperazine.

A salt of a compound can be made by reacting the appropriate compound in the form of the free base with the appropriate acid (or a free acid with a base).

The compounds disclosed herein, and their salts, can exist in one or more isomeric forms, for example in stereoisomeric (including diastereomeric, enantiomeric, and epimeric forms, as well as d-isomers and 1-isomers, and appropriate mixtures thereof), geometric (including cis, trans, syn, anti, entgegen (E), and zusammen (Z) isomers, and appropriate mixtures thereof), tautomeric, and polymorphic forms. Additionally, the compounds disclosed herein can exist in unsolvated as well as solvated forms with pharmaceutically acceptable solvents such as water, ethanol, and the like.

### EXAMPLES

Exemplary embodiments are provided in the following Examples 1-X. The following examples are presented only by way of illustration and to assist one of ordinary skill in using the invention. The examples are not intended in any way to otherwise limit the scope of the invention. In some embodiments, said IGF-1R inhibitor is an antibody or a subset of antibodies chosen from amongst the Examples below. In some embodiments, said IGF-1R inhibitor is a small molecule or a subset of small molecules chosen from amongst the Examples below.

### Example A

Teprotumumab

Provided first is teprotumumab (TEPEZZA), an IGF-1R inhibitor approved for the treatment of TED. Teprotumumab and other related IGF-1R inhibitor antibodies and their methods of preparation can be found in U.S. Pat. No. 7,572,897, US20190225696, and US20190270820, which are hereby incorporated by reference in their entireties. In certain embodiments, teprotumumab may be used as an active control in clinical trials of other IGF-1R inhibitors, e.g. as in Example 31.

TABLE A
Teprotumumab Sequences and SEQ ID Numbers

| SEQ | | |
|---|---|---|
| ID NO | Description | Sequence |
| | Antibody 1 | |
| 84 | CDRH1 aa | Ser Tyr Gly Met His |
| 85 | CDRH2 aa | Ile Ile Trp Phe Asp Gly Ser Ser Thr Tyr Tyr Ala Asp Ser Val |
| | | Arg Gly |
| 86 | CDRH3 aa | Glu Leu Gly Arg Arg Tyr Phe Asp Leu |
| 87 | CDRL1 aa | Arg Ala Ser Gln Ser Val Ser Ser Tyr Leu Ala |
| 88 | CDRL2 aa | Asp Ala Ser Lys Arg Ala Thr |
| 89 | CDRL3 aa | Gln Gln Arg Ser Lys Trp Pro Pro Trp Thr |
| 90 | VH aa | Gln Val Glu Leu Val Glu Ser Gly Gly Gly Val Val Gln Pro Gly |
| | | Arg Ser Gln Arg Leu Ser Cys Ala Ala Ser Gly Phe Thr Phe Ser |
| | | Ser Tyr Gly Met His Trp Val Arg Gln Ala Pro Gly Lys Gly Leu |
| | | Glu Trp Val Ala Ile Ile Trp Phe Asp Gly Ser Ser Thr Tyr Tyr |
| | | Ala Asp Ser Val Arg Gly Arg Phe Thr Ile Ser Arg Asp Asn Ser |
| | | Lys Asn Thr Leu Tyr Leu Gln Met Asn Ser Leu Arg Ala Glu |
| | | Asp Thr Ala Val Tyr Phe Cys Ala Arg Glu Leu Gly Arg Arg |
| | | Tyr Phe Asp Leu Trp Gly Arg Gly Thr Leu Val Ser Val Ser Ser |
| 91 | VL aa | Glu Ile Val Leu Thr Gln Ser Pro Ala Thr Leu Ser Leu Ser Pro |
| | | Gly Glu Arg Ala Thr Leu Ser Cys Arg Ala Ser Gln Ser Val Ser |
| | | Ser Tyr Leu Ala Trp Tyr Gln Gln Lys Pro Gly Gln Ala Pro Arg |
| | | Leu Leu Ile Tyr Asp Ala Ser Lys Arg Ala Thr Gly Ile Pro Ala |
| | | Arg Phe Ser Gly Ser Gly Ser Gly Thr Asp Phe Thr Leu Thr Ile |
| | | Ser Ser Leu Glu Pro Glu Asp Phe Ala Val Tyr Tyr Cys Gln Gln |
| | | Arg Ser Lys Trp Pro Pro Trp Thr Phe Gly Gln Gly Thr Lys Val |
| | | Glu Ser Lys |
| | Antibody 2 | |
| 84 | CDRH1 aa | Ser Tyr Gly Met His |
| 92 | CDRH2 aa | Ile Ile Trp Phe Asp Gly Ser Ser Lys Tyr Tyr Gly Asp Ser Val |
| | | Lys Gly |
| 86 | CDRH3 aa | Glu Leu Gly Arg Arg Tyr Phe Asp Leu |
| 87 | CDRL1 aa | Arg Ala Ser Gln Ser Val Ser Ser Tyr Leu Ala |
| 93 | CDRL2 aa | Asp Ala Ser Asn Arg Ala Thr |
| 89 | CDRL3 aa | Gln Gln Arg Ser Lys Trp Pro Pro Trp Thr |
| 94 | VH aa | Gin Val Gin Leu Val Glu Ser Gly Gly Gly Val Val Gin Pro Gly |
| | | Arg Ser Leu Arg Leu Ser Cys Ala Ala Ser Gly Phe Thr Phe Ser |
| | | Ser Tyr Gly Met His Trp Val Arg Gin Ala Pro Gly Lvs Gly Leu |
| | | Glu Trp Met Ala Ile Ile Trp Phe Asp Gly Ser Ser Lvs Tyr Tyr |
| | | Gly Asp Ser Val Lvs Gly Arg Phe Thr Ile Ser Arg Asp Asn Ser |
| | | Lys Asn Thr Leu Tyr Leu Gin Met Asn Ser Leu Arg Ala Asp |
| | | Asp Thr Ala Val Tyr Tyr Cys Ala Arg Glu Leu Gly Arg Arg |
| | | Tyr Phe Asp Leu Trp Gly Arg Gly Thr Leu Val Thr Val Ser Ser |
| 95 | VL aa | Glu Ile Val Leu Thr Gin Ser Pro Ala Thr Leu Ser Leu Ser Pro |
| | | Gly Glu Arg Ala Thr Leu Ser Cys Arg Ala Ser Gin Ser Val Ser |
| | | Ser Tyr Leu Ala Trp Tyr Gin Gin Lvs Pro Gly Gin Ala Pro Arg |
| | | Leu Leu Ile Tyr Asp Ala Ser Asn Arg Ala Thr Gly Ile Pro Ala |
| | | Arg Phe Ser Gly Ser Gly Ser Gly Thr Asp Phe Thr Leu Thr Ile |
| | | Ser Ser Leu Glu Pro Glu Asp Phe Ala Val Tyr Tyr Cys Gin Gin |
| | | Arg Ser Lys Trp Pro Pro Trp Thr Phe Gly Gin Gly Thr Lys Val |
| | | Glu Ile Lys |

### Example 1

Dalotuzumab

Dalotuzumab and other related IGF-1R inhibitor antibodies and their methods of preparation can be found in WO 2005/058967, which is hereby incorporated by reference in its entirety.

Heavy Chain CDRs—Dalotuzumab

| HCDR1 | HCDR2 | HCDR3 |
|---|---|---|
| GGYLWN | YISYDGTN | YGRVFFDY |
| (SEQ ID | NYKPSLKD | (SEQ ID |
| NO: 1) | (SEQ ID | NO: 3) |
| | NO: 2) | |

Light Chain CDRs—Dalotuzumab

| LCDR1 | LCDR2 | LCDR3 |
|---|---|---|
| RSSQSIVHS | KVSNRLY | FQGSHVPWT |
| NGNTYLQ | (SEQ ID | (SEQ ID |
| (SEQ ID | NO: 5) | NO: 6) |
| NO: 4) | | |

| | |
|---|---|
| Heavy Chain | QVQLQESGPGLVKPSETLSLTCTVS |
| (HC) | GYSITGGYLWNWIRQPPGKGLEWIG |
| | YISYDGTNNYKPSLKDRVTISRDTS |
| | KNQFSLKLSSVTAADTAVYYCARYG |
| | RVFFDYWGQGTLVTVSSASTKGPSV |
| | FPLAPSSKSTSGGTAALGCLVKDYF |
| | PEPVTVSWNSGALTSGVHTFPAVLQ |
| | SSGLYSLSSVVTVPSSSLGTQTYIC |
| | NVNHKPSNTKVDKRVEPKSCDKTHT |
| | CPPCPAPELLGGPSVFLFPPKPKDT |
| | LMISRTPEVTCVVVDVSHEDPEVKF |
| | NWYVDGVEVHNAKTKPREEQYNSTY |
| | RVVSVLTVLHQDWLNGKEYKCKVSN |
| | KALPAPIEKTISKAKGQPREPQVYT |
| | LPPSREEMTKNQVSLTCLVKGFYPS |
| | DIAVEWESNGQPENNYKTTPPVLDS |
| | DGSFFLYSKLTVDKSRWQQGNVFSC |
| | SVMHEALHNHYTQKSLSLSPGK |
| | (SEQ ID NO: 7) |
| Light Chain | DIVMTQSPLSLPVTPGEPASISCRS |
| (LC) | SQSIVHSNGNTYLQWYLQKPGQSPQ |
| | LLIYKVSNRLYGVPDRFSGSGSGTD |
| | FTLKISRVEAEDVGVYYCFQGSHVP |
| | WTFGQGTKVEIKRTVAAPSVFIFPP |
| | SDEQLKSGTASVVCLLNNFYPREAK |
| | VQWKVDNALQSGNSQESVTEQDSKD |
| | STYSLSSTLTLSKADYEKHKVYACE |
| | VTHQGLSSPVTKSFNRGEC |
| | (SEQ ID NO: 8) |

Some embodiments of the disclosure are anti-IGF-1R inhibitor mAbs or antigen binding fragments thereof, comprising a heavy chain comprising a variable heavy chain CDR1, a variable heavy chain CDR2, and a variable heavy chain CDR3, wherein the variable heavy chain CDR1 comprises an amino acid sequence SEQ ID NO:1, the variable heavy chain CDR2 comprises an amino acid sequence SEQ ID NO:2; and the variable heavy chain CDR3 comprises an amino acid sequence SEQ ID NO:3 or at least a CDR with at least 80% of sequence identity after optimal alignment with SEQ ID NO:1, SEQ ID NO:2, and SEQ ID NO:3.

The anti-IGF-1R inhibitor mAbs or antibody or antigen binding fragment thereof may additionally comprise a light chain which is paired with the heavy chain to form an antigen binding domain. In some embodiments, the light chain comprises a variable light chain CDR1, a variable light chain CDR2, and a variable light chain CDR3, wherein the variable light chain CDR1 comprises an amino acid sequence SEQ ID NO:4, the variable light chain CDR2 comprises an amino acid sequence SEQ ID NO:5; and the variable light chain CDR3 comprises an amino acid sequence SEQ ID NO:6 or at least a CDR with at least 80% of homology after optimal alignment with SEQ ID NO:4, SEQ ID NO:5, and SEQ ID NO:6.

In some embodiments, the anti-IGF-1R inhibitor mAbs or antigen binding fragment thereof comprises a heavy chain amino acid sequence of SEQ ID NO:7 or at least a heavy chain with at least 85%, 90%, 95%, 97%, 98%, or 99% of sequence identity after optimal alignment with SEQ ID NO:7. Alternatively, or in addition, the anti-IGF-1R inhibitor mAbs or antigen binding fragment thereof may comprise a light chain having an amino acid sequence of SEQ ID NO:8 or at least a heavy chain with at least 85%, 90%, 95%, 97%, 98%, or 99% of sequence identity after optimal alignment with SEQ ID NO:8.

### Example 2

Ganitumab

Ganitumab and other related IGF-1R inhibitor antibodies and their methods of preparation can be found in WO 2006/069202, which is hereby incorporated by reference in its entirety.

Heavy Chain CDRs—Ganitumab

| HCDR1 | HCDR2 | HCDR3 |
|---|---|---|
| SSNWWS | EIYHSGST | WTGRT |
| (SEQ ID | NYNPSLKS | DAFDI |
| NO: 9) | (SEQ ID | (SEQ ID |
| | NO: 10) | NO: 11) |

Light Chain CDRs—Ganitumab

| LCDR1 | LCDR2 | LCDR3 |
|---|---|---|
| ISCRSSQSLL | LGSNRAS | MQGTHWPLT |
| HSNGYNYLD | (SEQ ID | (SEQ ID |
| (SEQ ID | NO: 13) | NO: 14) |
| NO: 12) | | |

| | |
|---|---|
| Heavy | QVQLQESGPGLVKPSGTLSLTCAVSGGSISSSNWWSWVRQPP |
| Chain | GKGLEWIGEIYHSGSTNYNPSLKSRVTSVDKSKNQFSLKLSS |
| (HC) | VTAADTAVYYCARWTGRTDAFDIWGQGTMVTVSSASTKGPSV |
| | FPLAPSSKSTSGGTAALGCLVKDYFPEPVTVSWNSGALTSGV |
| | HTFPAVLQSSGLYSLSSVVTVPSSSLGTQTYICNVNHKPSN |
| | TKVDKKVEPKSCDKTHTCPPCPAPELLGGPSVFLFPPKPK |
| | DTLMISRTPE VTCVVVDVSH EDPEVKFNWYVDGVEVHNA |
| | KTKPREEQYNSTYRVVSVLTVLHQDWLNGKEYKCKVSNKAL |
| | PAPIEKTISKAKGQPREPQVYTLPPSRDELTKNQVSLTCL |
| | VKGFYPSDIAVEWESNGQPE NNYKTTPPVLDSDGSFFLYS |
| | KLTVDKSRWQQGNVFSCSVM HEALHNHYTQ KSLSLSPGK |
| | (SEQ ID NO: 15) |
| Light | DVVMTQSPLS LPVTPGEPASISCRSSQSLLHSNGYNYLDW |
| Chain | YLQKPGQSPQLLIYLGSNRASGVPDRFSGSGSGTDFTLKI |
| (LC) | SRVEAEDVGVYYCMQGTHWPLTFGQGTKVE IKRTVAAPSV |
| | FIFPPSDEQL KSGTASVVCL LNNFYPREAKVQWKVDNAL |
| | QSGNSQESVTE QDSKDSTYSLSSTLTLSKADYEKHKVYAC |
| | EVTHQGLSSPVTKSFNRGEC (SEQ ID NO: 16) |

Some embodiments of the disclosure are anti-IGF-1R inhibitor mAbs or antigen binding fragments thereof, comprising a heavy chain comprising a variable heavy chain CDR1, a variable heavy chain CDR2, and a variable heavy chain CDR3, wherein the variable heavy chain CDR1 comprises an amino acid sequence SEQ ID NO:9, the variable heavy chain CDR2 comprises an amino acid sequence SEQ ID NO:10; and the variable heavy chain CDR3 comprises an amino acid sequence SEQ ID NO:11 or at least a CDR with at least 80% of sequence identity after optimal alignment with SEQ ID NO:9, SEQ ID NO:10, and SEQ ID NO:11.

The anti-IGF-1R inhibitor mAbs or antibody or antigen binding fragment thereof may additionally comprise a light chain which is paired with the heavy chain to form an antigen binding domain. In some embodiments, the light chain comprises a variable light chain CDR1, a variable light chain CDR2, and a variable light chain CDR3, wherein the variable light chain CDR1 comprises an amino acid sequence SEQ ID NO:12, the variable light chain CDR2 comprises an amino acid sequence SEQ ID NO:13; and the variable light chain CDR3 comprises an amino acid sequence SEQ ID NO:14 or at least a CDR with at least 80% of homology after optimal alignment with SEQ ID NO:12, SEQ ID NO:13, and SEQ ID NO:14.

In some embodiments, the anti-IGF-1R inhibitor mAbs or antigen binding fragment thereof comprises a heavy chain amino acid sequence of SEQ ID NO:15 or at least a heavy chain with at least 85%, 90%, 95%, 97%, 98%, or 99% of sequence identity after optimal alignment with SEQ ID NO:15. Alternatively, or in addition, the anti-IGF-1R inhibitor mAbs or antigen binding fragment thereof may comprise a light chain having an amino acid sequence of SEQ ID NO:16 or at least a heavy chain with at least 85%, 90%, 95%, 97%, 98%, or 99% of sequence identity after optimal alignment with SEQ ID NO:16.

### Example 3

Xentuzumab

Xentuzumab and other related IGF-1R inhibitor antibodies and their methods of preparation can be found in WO 2014/135611, which is hereby incorporated by reference in its entirety.

Heavy Chain CDRs—Xentuzumab

| HCDR1 | HCDR2 | HCDR3 |
|---|---|---|
| SYWMS | SITSYGSF | NMYTHFDS |
| (SEQ ID | TYADSVK | (SEQ ID |
| NO: 17) | (SEQ ID | NO: 19) |
| | NO: 18) | |

Light Chain CDRs—Xentuzumab

| LCDR1 | LCDR2 | LCDR3 |
|---|---|---|
| SGSSSN | DNSKRPS | QSRDTY |
| IGSNSVS | (SEQ ID | GYYWV |
| (SEQ ID | NO: 21) | (SEQ ID |
| NO: 20) | | NO: 22) |
| QSRDTY | | |
| GYYWV | | |

| | |
|---|---|
| Heavy | QVELVESGGGLVQPGGSLRLSCAASGFTFTSYWMSWVRQA |
| Chain | PGKGLELVSSITSYGSFTYYADSVKGRFTISRDNSKNTLY |
| (HC) | LQMNSLRAEDTAVYYCARNMYTHFDSWGQGTLVTVSSAST |
| | KGPSVFPLAPSSKSTSGGTAALGCLVKDYFPEPVTVSWNS |
| | GALTSGVHTF PAVLQSSGLYSLSSVVTVPS SSLGTQTY |
| | ICNVNHKPSNTKVDKKVEPKSCDKTHTCPPCPAPELLGGPS |
| | VFLFPPKPKDTLMISRTPEVTCVVVDVSHEDPEVKFNWYVD |
| | GVEVHNAKTK PREEQYNSTYRVVSVLTVLHQDWLNGKEYK |
| | CKVSNKALPAPIEKTISKAKGQPREPQVYTLPPSRDELTK |
| | NQVSLTCLVK GFYPSDIAVEWESNGQPENNYKTTPPVLDS |
| | (SEQ ID NO: 23) |
| Light | DIVLTQPPSVSGAPGQRVTISCSGSSSNIGSNSVSWYQQL |
| Chain | PGTAPKLLIYDNSKRPSGVPDRFSGSKSGTSASLAITGLQ |
| (LC) | SEDEADYYCQSRDTYGYYWVFGGGTKLTVLGQPKAAPSVT |
| | LFPPSSEELQANKATLVCLI SDFYPGAVTVAWKGDSSPV |
| | KAGVETTTPSKQSNNKYAASSYLSLTPEQWKSHRSYSCQV |
| | THEGSTVEKTVAPTECS (SEQ ID NO: 24) |

Some embodiments of the disclosure are anti-IGF-1R inhibitor mAbs or antigen binding fragments thereof, comprising a heavy chain comprising a variable heavy chain CDR1, a variable heavy chain CDR2, and a variable heavy chain CDR3, wherein the variable heavy chain CDR1 comprises an amino acid sequence SEQ ID NO:17, the variable heavy chain CDR2 comprises an amino acid sequence SEQ ID NO:18; and the variable heavy chain CDR3 comprises an amino acid sequence SEQ ID NO:19 or at least a CDR with at least 80% of sequence identity after optimal alignment with SEQ ID NO:17, SEQ ID NO:18, and SEQ ID NO:19.

The anti-IGF-1R inhibitor mAbs or antibody or antigen binding fragment thereof may additionally comprise a light chain which is paired with the heavy chain to form an antigen binding domain. In some embodiments, the light chain comprises a variable light chain CDR1, a variable light chain CDR2, and a variable light chain CDR3, wherein the variable light chain CDR1 comprises an amino acid sequence SEQ ID NO:20, the variable light chain CDR2 comprises an amino acid sequence SEQ ID NO:21; and the variable light chain CDR3 comprises an amino acid sequence SEQ ID NO:22 or at least a CDR with at least 80% of homology after optimal alignment with SEQ ID NO:20, SEQ ID NO:21, and SEQ ID NO:22.

In some embodiments, the anti-IGF-1R inhibitor mAbs or antigen binding fragment thereof comprises a heavy chain amino acid sequence of SEQ ID NO:23 or at least a heavy chain with at least 85%, 90%, 95%, 97%, 98%, or 99% of sequence identity after optimal alignment with SEQ ID NO:23. Alternatively, or in addition, the anti-IGF-1R inhibitor mAbs or antigen binding fragment thereof may comprise a light chain having an amino acid sequence of SEQ ID NO:24 or at least a heavy chain with at least 85%, 90%, 95%, 97%, 98%, or 99% of sequence identity after optimal alignment with SEQ ID NO:24.

### Example 4

AVE1642

AVE1642 and other related IGF-1R inhibitor antibodies and their methods of preparation can be found in WO 2003/106621, which is hereby incorporated by reference in its entirety.

Heavy Chain CDRs—AVE1642

| HCDR1 | HCDR2 | HCDR3 |
|---|---|---|
| SYWMH | EINPSNGRT | GRPDYYGS |
| (SEQ ID | NYNEKFKR | SKWYFDV |
| NO: 25) | (SEQ ID | (SEQ ID |
| | NO: 26) | NO: 27) |
| GYTFTSYWMH | EINPSNGRTN | GRPDYYGS |
| (SEQ ID | (SEQ ID | SKWYFDV |
| NO: 75) | NO: 76) | (SEQ ID |
| | | NO: 27) |
| SYWMH | EINPSNGRTN | GRPDYYGS |
| (SEQ ID | (SEQ ID | SKWYFDV |
| NO: 25) | NO: 76) | (SEQ ID |
| | | NO: 27) |
| SYWMH | EINPSNGRTN | GRPDYYGS |
| (SEQ ID | YNQKFQG | SKWYFDV |
| NO: 25) | (SEQ ID | (SEQ ID |
| | NO: 77) | NO: 27) |
| GYTFTSYWMH | EINPSNGRTN | GRPDYYGS |
| (SEQ ID | YNQKFQG | SKWYFDV |
| NO: 75) | (SEQ ID | (SEQ ID |
| | NO: 77) | NO: 27) |

Light Chain CDRs—AVE1642

| LCDR1 | LCDR2 | LCDR3 |
|---|---|---|
| RSSQSIVHS | KVSNRFS | FQGSHVPPT |
| NVNTYLE | (SEQ ID | (SEQ ID |
| (SEQ ID | NO: 29) | NO: 30) |
| NO: 28) | | |

Variable Domains—AVE1642

| | |
|---|---|
| Heavy Chain | QVQLQQSGAELVKPGASVKL |
| (VH1) | SCKASGYTFTSYWMHWVKQR |
| | PGQGLEWIGEINPSNGRTNY |
| | NEKFKRKATLTVDKSSSTAY |
| | MQLSSLTSEDSAVYYFARGR |
| | PDYYGSSKWYFDVWGAGTTV |
| | TVSS |
| | (SEQ ID NO: 31) |
| Heavy Chain | QVQLVQSGAEVVKPGASVKL |
| (VH2) | SCKASGYTFTSYWMHWVKQR |
| | PGQGLEWIGEINPSNGRTNY |
| | NQKFQGKATLTVDKSSSTAY |
| | MQLSSLTSEDSAVYYFARGR |
| | PDYYGSSKWYFDVWGQGTTV |
| | TVSS |
| | (SEQ ID NO: 78) |
| Heavy Chain | QVQLVQSGAEVVKPGASVKL |
| (VH3) | SCKASGYTFTSYWMHWVKQR |
| | PGQGLEWIGEINPSNGRTNY |
| | NQKFQGKATLTVDKSSSTAY |
| | MQLSSLTSEDSAVYYFARGR |
| | PDYYGSSKWYFDVWGQGTTV |
| | TVS |
| | (SEQ ID NO: 79) |
| Light Chain | DVLMTQTPLSLPVSLGDQAS |
| (VL1) | ISCRSSQSIVHSNVNTYLEW |
| | YLQKPGQSPKLLIYKVSNRF |
| | SGVPDRFSGSGSGTDFTLRI |
| | SRVEAEDLGIYYCFQGSHVP |
| | PTFGGGTKLEIKR |
| | (SEQ ID NO: 32) |
| Light Chain | DVVMTQTPLSLPVSLGDPAS |
| (VL2) | ISCRSSQSIVHSNVNTYLEW |
| | YLQKPGQSPRLLIYKVSNRF |
| | SGVPDRFSGSGAGTDFTLRI |
| | SRVEAEDLGIYYCFQGSHVP |
| | PTFGGGTKLEIKR |
| | (SEQ ID NO: 80) |
| Light Chain | DVLMTQTPLSLPVSLGDPAS |
| (VL3) | ISCRSSQSIVHSNVNTYLEW |
| | YLQKPGQSPKLLIYKVSNRF |
| | SGVPDRFSGSGAGTDFTLRI |
| | SRVEAEDLGIYYCFQGSHVP |
| | PTFGGGTKLEIKR |
| | (SEQ ID NO: 81) |
| Light Chain | DVLMTQTPLSLPVSLGDPAS |
| (VL4) | ISCRSSQSIVHSNVNTYLEW |
| | YLQKPGQSPRLLIYKVSNRF |
| | SGVPDRFSGSGAGTDFTLRI |
| | SRVEAEDLGIYYCFQGSHVP |
| | PTFGGGTKLEIKR |
| | (SEQ ID NO: 82) |
| Light Chain | DVVMTQTPLSLPVSLGDPAS |
| (VL5) | ISCRSSQSIVHSNVNTYLEW |
| | YLQKPGQSPKLLIYKVSNRF |
| | SGVPDRFSGSGAGTDFTLRI |
| | SRVEAEDLGIYYCFQGSHVP |
| | PTFGGGTKLEIKR |
| | (SEQ ID NO: 83) |

Some embodiments of the disclosure are anti-IGF-1R inhibitor mAbs or antigen binding fragments thereof, comprising a heavy chain comprising a variable heavy chain CDR1, a variable heavy chain CDR2, and a variable heavy chain CDR3, wherein the variable heavy chain CDR1 comprises an amino acid sequence SEQ ID NO:25, the variable heavy chain CDR2 comprises an amino acid sequence SEQ ID NO:26; and the variable heavy chain CDR3 comprises an amino acid sequence SEQ ID NO:27 or at least a CDR with at least 80% of sequence identity after optimal alignment with SEQ ID NO:25, SEQ ID NO:26, and SEQ ID NO:27.

The anti-IGF-1R inhibitor mAbs or antibody or antigen binding fragment thereof may additionally comprise a light chain which is paired with the heavy chain to form an antigen binding domain. In some embodiments, the light chain comprises a variable light chain CDR1, a variable light chain CDR2, and a variable light chain CDR3, wherein the variable light chain CDR1 comprises an amino acid sequence SEQ ID NO:28, the variable light chain CDR2 comprises an amino acid sequence SEQ ID NO:29; and the variable light chain CDR3 comprises an amino acid sequence SEQ ID NO:30 or at least a CDR with at least 80% of homology after optimal alignment with SEQ ID NO:28, SEQ ID NO:29, and SEQ ID NO:30.

In some embodiments, the anti-IGF-1R inhibitor mAbs or antigen binding fragment thereof comprises a heavy chain amino acid sequence of SEQ ID NO:31 or at least a heavy chain with at least 85%, 90%, 95%, 97%, 98%, or 99% of sequence identity after optimal alignment with SEQ ID NOs:31, 78, or 79. Alternatively, or in addition, the anti-IGF-1R inhibitor mAbs or antigen binding fragment thereof may comprise a light chain having an amino acid sequence of SEQ ID NO:32 or at least a heavy chain with at least 85%, 90%, 95%, 97%, 98%, or 99% of sequence identity after optimal alignment with SEQ ID NOs:32, 80, 81, 82, or 83.

### Example 5

Figitumumab

Figitumumab and other related IGF-1R inhibitor antibodies and their methods of preparation can be found in U.S. Pat. No. 7,037,498 which is hereby incorporated by reference in its entirety.

Heavy Chain CDRs—Figitumumab

| HCDR1 | HCDR2 | HCDR3 |
|---|---|---|
| GFTFSSYAMN | AISGSGGTT | DLGWSDSYY |
| (SEQ ID | FYADSVKG | YYYGMDV |
| NO: 33) | (SEQ ID | (SEQ ID |
| | NO: 34) | NO: 35) |

Light Chain CDRs—Figitumumab

| LCDR1 | LCDR2 | LCDR3 |
|---|---|---|
| RASQGIRNDLG | AASRLHR | LQHNSYPCS |
| SEQ ID NO: 36) | (SEQ ID NO: 37) | (SEQ ID NO: 38) |

| | |
|---|---|
| Heavy Chain (HC) | EVQLLESGGGLVQPGGSLRL |
| | SCTASGFTFSSYAMNWVRQA |
| | PGKGLEWVSAISGSGGTTFY |
| | ADSVKGRFTISRDNSRTTLY |
| | LQMNSLRAEDTAVYYCAKDL |
| | GWSDSYYYYYGMDVWGQGTT |
| | VTVSSASTKGPSVFPLAPCS |
| | RSTSESTAALGCLVKDYFP |
| | EPVTVSWNSGALTSGVHTFP |
| | AVLQSSGLYSLSSVVTVPSS |
| | NFGTQTYTCNVDHKPSNTKVD |
| | KTVERKCCVECPPCPAPP |
| | VAGPSVFLFPPKPKDTLMIS |
| | RTPEVTCVVVDVSHEDPEVQ |
| | FNWYVDGVEVHNAKTKPREE |
| | QFNSTFRVVSVLTVVHQDWL |
| | NGKEYKCKVSNKGLPAPIEKTI |
| | SKTKGQPREPQVYTLPP |
| | SREEMTKNQVSLTCLVKGFY |
| | PSDIAVEWESNGQPENNYKT |
| | TPPMLDSDGSFFLYSKLTVD |
| | KSRWQQGNVFSCSVMHEALH |
| | NHYTQKSLSLSPGK |
| | (SEQ ID NO: 39) |
| Light Chain (LC) | DIQMTQFPSSLSASVGDRVT |
| | ITCRASQGIRNDLGWYQQKP |
| | GKAP |
| | KRLIYAASRLHRGVPSRFSG |
| | SGSGTEFTLTISSLQPEDFA |
| | TYYCLQHNSYPCSFGQGTKL |
| | EIKRTVAAPSVFIFPPSDEQ |
| | LKSGTASVVCLLNNFYPREA |
| | KVQWKVDNALQSGNSQESVT |
| | EQDSKDSTYSLSSTLTLSKA |
| | DYEKHKVYACEVTHQGLSSP |
| | VTKSFNRGEC |
| | (SEQ ID NO: 40) |

Some embodiments of the disclosure are anti-IGF-1R inhibitor mAbs or antigen binding fragments thereof, comprising a heavy chain comprising a variable heavy chain CDR1, a variable heavy chain CDR2, and a variable heavy chain CDR3, wherein the variable heavy chain CDR1 comprises an amino acid sequence SEQ ID NO:33, the variable heavy chain CDR2 comprises an amino acid sequence SEQ ID NO:34; and the variable heavy chain CDR3 comprises an amino acid sequence SEQ ID NO:35 or at least a CDR with at least 80% of sequence identity after optimal alignment with SEQ ID NO:33, SEQ ID NO:34, and SEQ ID NO:35.

The anti-IGF-1R inhibitor mAbs or antibody or antigen binding fragment thereof may additionally comprise a light chain which is paired with the heavy chain to form an antigen binding domain. In some embodiments, the light chain comprises a variable light chain CDR1, a variable light chain CDR2, and a variable light chain CDR3, wherein the variable light chain CDR1 comprises an amino acid sequence SEQ ID NO:36, the variable light chain CDR2 comprises an amino acid sequence SEQ ID NO:37; and the variable light chain CDR3 comprises an amino acid sequence SEQ ID NO:38 or at least a CDR with at least 80% of homology after optimal alignment with SEQ ID NO:36, SEQ ID NO:37, and SEQ ID NO:38.

In some embodiments, the anti-IGF-1R inhibitor mAbs or antigen binding fragment thereof comprises a heavy chain amino acid sequence of SEQ ID NO:39 or at least a heavy chain with at least 85%, 90%, 95%, 97%, 98%, or 99% of sequence identity after optimal alignment with SEQ ID NO:39. Alternatively, or in addition, the anti-IGF-1R inhibitor mAbs or antigen binding fragment thereof may comprise a light chain having an amino acid sequence of SEQ ID NO:40 or at least a heavy chain with at least 85%, 90%, 95%, 97%, 98%, or 99% of sequence identity after optimal alignment with SEQ ID NO:40.

### Example 6

Dusigitumab

Dusigitumab (MEDI-573) and other related IGF-1R inhibitor antibodies and their methods of preparation can be found in U.S. Pat. No. 7,939,637 which is hereby incorporated by reference in its entirety.

Heavy Chain CDRs—Dusigitumab

| HCDR1 | HCDR2 | HCDR3 |
|---|---|---|
| SYDIN | WMNPNSGNT | DPYYY |
| (SEQ ID | GYAQKFQG | YYGMDV |
| NO: 41) | (SEQ ID | (SEQ ID |
| | NO: 42) | NO: 43) |

Light Chain CDRs—Dusigitumab

| | | |
|---|---|---|
| LCDR1 | LCDR2 | LCDR3 |
| SGSSSNI | DNNKRPS | ETWDT |
| ENNHVS | (SEQ ID | SLSAGRV |
| (SEQ ID | NO: 45) | (SEQ ID |
| NO: 44) | | NO: 46) |

| | |
|---|---|
| Heavy Chain (HC) | QVQLVQSGAEVKKPGASVKV |
| | SCKASGYTFTSYDINWVRQA |
| | TGQGLEWMGWMNPNSGNTGY |
| | AQKFQGRVTMTRNTSISTAY |
| | MELSSLRSEDTAVYYCARDP |
| | YYYYYGMDVWGQGTTVTVSS |
| | ASTKGPSVFPLAPCSRSTSE |
| | STAALGCLVKDYFPEPVTVS |
| | WNSGALTSGVHTFPAVLQSS |
| | GLYSLSSVVTVPSSNFGTQT |
| | YTCNVDHKPSNTKVDKTVER |
| | KCCVECPPCPAPPVAGPSVF |
| | LFPPKPKDTLMISRTPEVTC |
| | VVVDVSHEDPEVQFNWYVDG |
| | VEVHNAKTKPREEQFNSTFR |
| | VVSVLTVVHQDWLNGKEYKC |
| | KVSNKGLPAPIEKTISKTKG |
| | QPREPQVYTLPPSREEMTKN |
| | QVSLTCLVKGFYPSDIAVEW |
| | ESNGQPENNYKTTPPMLDSD |
| | GSFFLYSKLTVDKSRWQQGN |
| | VFSCSVMHEALHNHYTQKSL |
| | SLSPGK |
| | (SEQ ID NO: 47) |
| Light Chain (LC) | QSVLTQPPSVSAAPGQKVTI |
| | SCSGSSSNIENNHVSWYQQL |
| | PGTAPKLLIYDNNKRPSGIP |
| | DRFSGSKSGTSATLGITGLQ |
| | TGDEADYYCETWDTSLSAGR |
| | VFGGGTKLTVLGQPKAAPSV |
| | TLFPPSSEELQANKATLVCL |
| | ISDFYPGAVTVAWKADSSPV |
| | KAGVETTTPSKQSNNKYAAS |
| | SYLSLTPEQWKSHRSYSCQV |
| | THEGSTVEKTVAPTECS |
| | (SEQ ID NO: 48) |

Some embodiments of the disclosure are anti-IGF-1R inhibitor mAbs or antigen binding fragments thereof, comprising a heavy chain comprising a variable heavy chain CDR1, a variable heavy chain CDR2, and a variable heavy chain CDR3, wherein the variable heavy chain CDR1 comprises an amino acid sequence SEQ ID NO:41, the variable heavy chain CDR2 comprises an amino acid sequence SEQ ID NO:42; and the variable heavy chain CDR3 comprises an amino acid sequence SEQ ID NO:43 or at least a CDR with at least 80% of sequence identity after optimal alignment with SEQ ID NO:41, SEQ ID NO:42, and SEQ ID NO:43.

The anti-IGF-1R inhibitor mAbs or antibody or antigen binding fragment thereof may additionally comprise a light chain which is paired with the heavy chain to form an antigen binding domain. In some embodiments, the light chain comprises a variable light chain CDR1, a variable light chain CDR2, and a variable light chain CDR3, wherein the variable light chain CDR1 comprises an amino acid sequence SEQ ID NO:44, the variable light chain CDR2 comprises an amino acid sequence SEQ ID NO:45; and the variable light chain CDR3 comprises an amino acid sequence SEQ ID NO:46 or at least a CDR with at least 80% of homology after optimal alignment with SEQ ID NO:44, SEQ ID NO:45, and SEQ ID NO:46.

In some embodiments, the anti-IGF-1R inhibitor mAbs or antigen binding fragment thereof comprises a heavy chain amino acid sequence of SEQ ID NO:39 or at least a heavy chain with at least 85%, 90%, 95%, 97%, 98%, or 99% of sequence identity after optimal alignment with SEQ ID NO:47. Alternatively, or in addition, the anti-IGF-1R inhibitor mAbs or antigen binding fragment thereof may comprise a light chain having an amino acid sequence of SEQ ID NO:40 or at least a heavy chain with at least 85%, 90%, 95%, 97%, 98%, or 99% of sequence identity after optimal alignment with SEQ ID NO:48.

### Example 7

Cixutumumab

Cixutumumab and other related IGF-1R inhibitor antibodies and their methods of preparation can be found in U.S. Pat. No. 7,638,605 which is hereby incorporated by reference in its entirety.

Heavy Chain CDRs—Cixutumumab

| HCDR1 | HCDR2 | HCDR3 |
|---|---|---|
| SYAIS | GIIPIFGTA | APLRFLEWSTQ |
| (SEQ ID | NYAQKFQ | DHYYYYYMDV |
| NO: 49) | (SEQ ID | (SEQ ID |
| | NO: 50) | NO: 51) |

Light Chain CDRs—Cixutumumab

| LCDR1 | LCDR2 | LCDR3 |
|---|---|---|
| QGDSLRSYYAT | GENKRPS | KSRDGSGQHLV |
| (SEQ ID | (SEQ ID | (SEQ ID |
| NO: 52) | NO: 53) | NO: 54) |

| | |
|---|---|
| Heavy Chain (HC) | EVQLVQSGAEVKKPGSSVKV |
| | SCKASGGTFSSYAISWVRQA |
| | PGQGLEWMGGIIPIFGTANY |
| | AQKFQGRVTITADKSTSTAY |
| | MELSSLRSEDTAVYYCARAP |
| | LRFLEWSTQDHYYYYYMDVW |
| | GKGTTVTVSSASTKGPSVFP |
| | LAPSSKSTSGGTAALGCLVK |
| | DYFPEPVTVSWNSGALTSGV |
| | HTFPAVLQSSGLYSLSSVVT |
| | VPSSSLGTQTYICNVNHKPS |
| | NTKVDKKVEPKSCDKTHTCP |
| | PCPAPELLGGPSVFLFPPKP |
| | KDTLMISRTPEVTCVVVDVS |
| | HEDPEVKFNWYVDGVEVHNA |
| | KTKPREEQYNSTYRVVSVLT |
| | VLHQDWLNGKEYKCKVSNKA |
| | LPAPIEKTISKAKGQPREPQ |
| | VYTLPPSREEMTKNQVSLTC |
| | LVKGFYPSDIAVEWESNGQP |
| | ENNYKTTPPVLDSDGSFFLY |
| | SKLTVDKSRWQQGNVFSCSV |
| | MHEALHNHYTQKSLSLSPGK |
| | (SEQ ID NO: 55) |
| Light Chain (LC) | SSELTQDPAVSVALGQTVRI |
| | TCQGDSLRSYYATWYQQKPG |
| | QAPILVIYGENKRPSGIPDR |
| | FSGSSSGNTASLTITGAQAE |
| | DEADYYCKSRDGSGQHLVFG |
| | GGTKLTVLGQ |
| | PKAAPSVTLF |
| | PPSSEELQANKATLVCLISDF |
| | YPGAVTVAWKADSSPVKAGV |
| | ETTTPSKQSNNKYAASSY |
| | LSLTPEQWKSHRSYSCQVTH |
| | EGSTVEKTVAP AECS |
| | (SEQ ID NO: 56) |

Some embodiments of the disclosure are anti-IGF-1R inhibitor mAbs or antigen binding fragments thereof, comprising a heavy chain comprising a variable heavy chain CDR1, a variable heavy chain CDR2, and a variable heavy chain CDR3, wherein the variable heavy chain CDR1 comprises an amino acid sequence SEQ ID NO:49, the variable heavy chain CDR2 comprises an amino acid sequence SEQ ID NO:50; and the variable heavy chain CDR3 comprises an amino acid sequence SEQ ID NO:51 or at least a CDR with at least 80% of sequence identity after optimal alignment with SEQ ID NO:49, SEQ ID NO:50, and SEQ ID NO:51.

The anti-IGF-1R inhibitor mAbs or antibody or antigen binding fragment thereof may additionally comprise a light chain which is paired with the heavy chain to form an antigen binding domain. In some embodiments, the light chain comprises a variable light chain CDR1, a variable light chain CDR2, and a variable light chain CDR3, wherein the variable light chain CDR1 comprises an amino acid sequence SEQ ID NO:52, the variable light chain CDR2 comprises an amino acid sequence SEQ ID NO:53; and the variable light chain CDR3 comprises an amino acid sequence SEQ ID NO:54 or at least a CDR with at least 80% of homology after optimal alignment with SEQ ID NO:52, SEQ ID NO:53, and SEQ ID NO:54.

In some embodiments, the anti-IGF-1R inhibitor mAbs or antigen binding fragment thereof comprises a heavy chain amino acid sequence of SEQ ID NO:55 or at least a heavy chain with at least 85%, 90%, 95%, 97%, 98%, or 99% of sequence identity after optimal alignment with SEQ ID NO:55. Alternatively, or in addition, the anti-IGF-1R inhibitor mAbs or antigen binding fragment thereof may comprise a light chain having an amino acid sequence of SEQ ID NO:56 or at least a heavy chain with at least 85%, 90%, 95%, 97%, 98%, or 99% of sequence identity after optimal alignment with SEQ ID NO:56.

### Example 8

BIIB022

BIIB022 and other related IGF-1R inhibitor antibodies and their methods of preparation can be found in U.S. Pat. No. 7,612,178 which is hereby incorporated by reference in its entirety.

Heavy Chain CDRs—BIIB022

| HCDR1 | HCDR2 | HCDR3 |
|---|---|---|
| IYRMQ | GISPSGGTT | WSGGSG |
| (SEQ ID | WYADSVKG | YAFDI |
| NO: 57) | (SEQ ID | (SEQ ID |
| | NO: 58) | NO: 59) |

Light Chain CDRs—BIIB022

| LCDR1 | LCDR2 | LCDR3 |
|---|---|---|
| QASRDIRNYN | DASSLQT | QQFDSLPHT |
| (SEQ ID | (SEQ ID | (SEQ ID |
| NO: 60) | NO: 61) | NO: 62) |

| | |
|---|---|
| Heavy Chain (HC) | EVQLLESGGGLVQPGGSLRLSC |
| | AASGFTFSIYRMQWVRQAPGKG |
| | LEWVSGISPSGGTTWYADSVKG |
| | RFTISRDNSKNTLYLQMNSLRA |
| | EDTAVYYCARWSGGSGYAFDIW |
| | GQGTMVTVSS |
| | (SEQ ID NO: 63) |
| Light Chain (LC) | DIQMTQSPLSLSASVGDRVTIT |
| | CQASRDIRNYLNWYQQKPGKAP |
| | KLLIYDASSLQTGVPSRFGGSG |
| | SGTDFSFTIGSLQPEDIATYYC |
| | QQFDSLPHTFGQGTKLEIK |
| | (SEQ ID NO: 64) |

Some embodiments of the disclosure are anti-IGF-1R inhibitor mAbs or antigen binding fragments thereof, comprising a heavy chain comprising a variable heavy chain CDR1, a variable heavy chain CDR2, and a variable heavy chain CDR3, wherein the variable heavy chain CDR1 comprises an amino acid sequence SEQ ID NO:57, the variable heavy chain CDR2 comprises an amino acid sequence SEQ ID NO:58; and the variable heavy chain CDR3 comprises an amino acid sequence SEQ ID NO:59 or at least a CDR with at least 80% of sequence identity after optimal alignment with SEQ ID NO:57, SEQ ID NO:58, and SEQ ID NO:59.

The anti-IGF-1R inhibitor mAbs or antibody or antigen binding fragment thereof may additionally comprise a light chain which is paired with the heavy chain to form an antigen binding domain. In some embodiments, the light chain comprises a variable light chain CDR1, a variable light chain CDR2, and a variable light chain CDR3, wherein the variable light chain CDR1 comprises an amino acid sequence SEQ ID NO:60, the variable light chain CDR2 comprises an amino acid sequence SEQ ID NO:61; and the variable light chain CDR3 comprises an amino acid sequence SEQ ID NO:62 or at least a CDR with at least 80% of homology after optimal alignment with SEQ ID NO:60, SEQ ID NO:61, and SEQ ID NO:62.

In some embodiments, the anti-IGF-1R inhibitor mAbs or antigen binding fragment thereof comprises a heavy chain amino acid sequence of SEQ ID NO:63 or at least a heavy chain with at least 85%, 90%, 95%, 97%, 98%, or 99% of sequence identity after optimal alignment with SEQ ID NO:63. Alternatively, or in addition, the anti-IGF-1R inhibitor mAbs or antigen binding fragment thereof may comprise a light chain having an amino acid sequence of SEQ ID NO:64 or at least a heavy chain with at least 85%, 90%, 95%, 97%, 98%, or 99% of sequence identity after optimal alignment with SEQ ID NO:64.

### Example 9

Robatumumab

Heavy Chain (HC) and Light Chain (LC) for Robatumumab

| | |
|---|---|
| Heavy Chain | EVQLVQSGGG LVKPGGSLRL SCAASGFTFS SFAMHWVRQA |
| (HC) | PGKGLEWISV IDTRGATYY ADSVKGRFTIS RDNAKNSLYL |
| | QMNSLRAEDT AVYYCARLGN FYYGMDVWGQ GTTVTVSSAS |
| | TKGPSVFPLA PSSKSTSGGT AALGCLVKDY FPEPVTVSWN |
| | SGALTSGVHT FPAVLQSSGLYSLSSVVTVP SSSLGTQTYI |
| | CNVNHKPSNT KVDKKVEPKS CDKTHTCPPC PAPELLGGPS |
| | VFLFPPKPKD TLMISRTPEV TCVVVDVSHE DPEVKFNWYV |
| | DGVEVHNAKT KPREEQYNSTYRVVSVLTVL HQDWLNGKEY |
| | KCKVSNKALP APIEKTISKA KGQPREPQVY TLPPSRDELT |
| | KNQVSLTCLV KGFYPSDIAV EWESNGQPEN NYKTTPPVLD |
| | SDGSFFLYSK LTVDKSRWQQGNVFSCSVMH EALHNHYTQK |
| | SLSLSPGK (SEQ ID NO: 65) |
| Light Chain | EIVLTQSPGTLSVSPGERATLSCRASQSIGSSLHWYQQKPGQAPR |
| (LC) | LLIKYASQSLSGIPDRFSGSGSGTDFTLTISRLEPEDFAVYYCHQS |
| | SRLPHTFGQGTKVEIKRTVAAPSVFIFPPSDEQLKSGTASVVCLL |
| | NNFYPREAKVQWKVDNALQSGNSQESVTEQDSKDSTYSLSSTL |
| | TLSKADYEKHKVYACEVTHQGLSSPVTKSFNRGEC |
| | (SEQ ID NO: 66) |

In some embodiments, the anti-IGF-1R inhibitor mAbs or antigen binding fragment thereof comprises a heavy chain amino acid sequence of SEQ ID NO:65 or at least a heavy chain with at least 85%, 90%, 95%, 97%, 98%, or 99% of sequence identity after optimal alignment with SEQ ID NO:65. Alternatively, or in addition, the anti-IGF-1R inhibitor mAbs or antigen binding fragment thereof may comprise a light chain having an amino acid sequence of SEQ ID NO:66 or at least a heavy chain with at least 85%, 90%, 95%, 97%, 98%, or 99% of sequence identity after optimal alignment with SEQ ID NO:66.

In some embodiments, said IGF-1R inhibitor is a small molecule.

### Example 10

Linsitinib

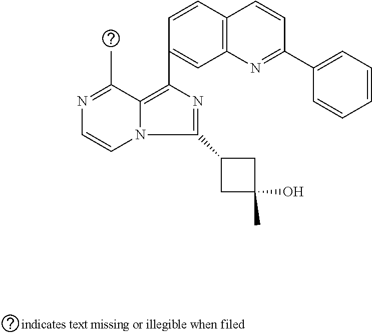 [SMILES: Cc1nccn2c1c(-c1ccc3ccc(-c4ccccc4)nc3c1)nc2[C@H]1C[C@@](C)(O)C1]

Linsitinib and other related IGF-1R inhibitor small molecules and their methods of preparation can be found in U.S. Pat. No. 8,101,613, which is hereby incorporated by reference in its entirety. Linsitinib and the other IGF-1R inhibitors described therein are predicted to have activity in the activity measures or assessments for the treatment of TED described herein.

### Example 11

Picropodophyllin

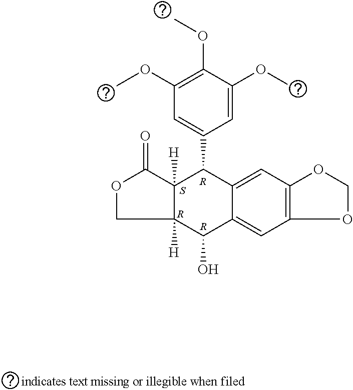

Picropodophyllin (AXL1717) and other related IGF-1R inhibitor small molecules and their methods of preparation can be found in US U.S. Pat. No. 4,567,253, which is hereby incorporated by reference in its entirety. Picropodophyllin and the other IGF-1R inhibitors described therein are predicted to have activity in the activity measures or assessments for the treatment of TED described herein.

### Example 12

GTX-134

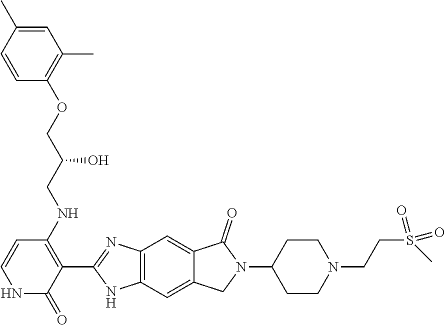 [SMILES: Cc1ccc(OC[C@H](O)CNc2cc[nH]c(=O)c2-c2nc3cc4c(cc3[nH]2)CN(C2CCN(CCS(C)(=O)=O)CC2)C4=O)c(C)c1]

GTX-134 and other related IGF-1R inhibitor small molecules and their methods of preparation can be found in U.S. Pat. No. 8,063,225, which is hereby incorporated by reference in its entirety. GTX-134 and the other IGF-1R inhibitors described therein are predicted to have activity in the activity measures or assessments for the treatment of TED described herein.

### Example 13

AG1024

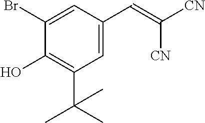

AG1024 and other related IGF-1R inhibitor small molecules and their methods of preparation can be found in WO1995024190, which is hereby incorporated by reference in its entirety. AG1024 and the other IGF-1R inhibitors described therein are predicted to have activity in the activity measures or assessments for the treatment of TED described herein.

### Example 14

BMS-536924

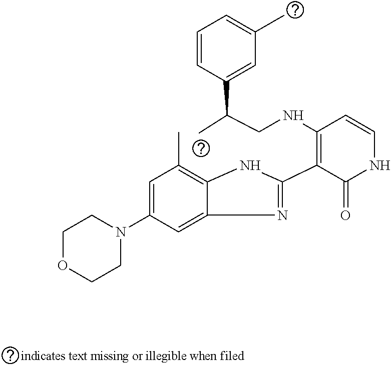 [SMILES: Cc1cccc([C@H](C)CNc2cc[nH]c(=O)c2-c2nc3cc(N4CCOCC4)cc(C)c3[nH]2)c1]

BMS-536924 and other related IGF-1R inhibitor small molecules and their methods of preparation can be found in U.S. Pat. No. 7,081,454, which is hereby incorporated by reference in its entirety. BMS-536924 and the other IGF-1R inhibitors described therein are predicted to have activity in the activity measures or assessments for the treatment of TED described herein.

### Example 15

NVP-AEW541

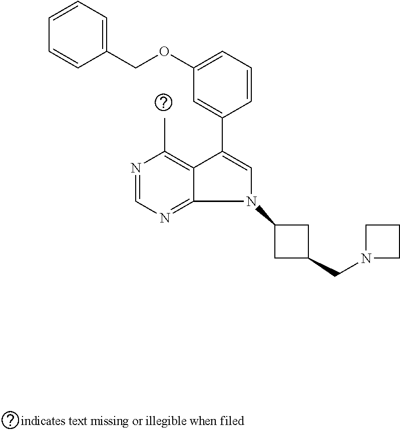 [SMILES: Cc1ncnc2c1c(-c1cccc(OCc3ccccc3)c1)cn2[C@H]1C[C@@H](CN2CCC2)C1]

NVP-AEW541 and other related IGF-1R inhibitor small molecules and their methods of preparation can be found in U.S. Pat. No. 7,326,699, which is hereby incorporated by reference in its entirety. NVP-AEW541 and the other IGF-1R inhibitors described therein are predicted to have activity in the activity measures or assessments for the treatment of TED described herein.

### Example 16

BMS-754807

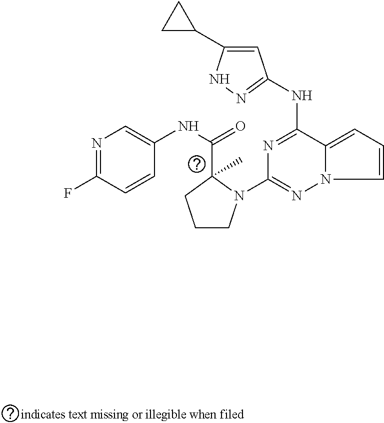 [SMILES: C[C@@]1(C(=O)Nc2ccc(F)nc2)CCCN1c1nc(Nc2cc(C3CC3)[nH]n2)c2cccn2n1]

BMS-754807 and other related IGF-1R inhibitor small molecules and their methods of preparation can be found in U.S. Pat. No. 7,534,792, which is hereby incorporated by reference in its entirety. BMS-754807 and the other IGF-1R inhibitors described therein are predicted to have activity in the activity measures or assessments for the treatment of TED described herein.

### Example 17

GSK1838705A

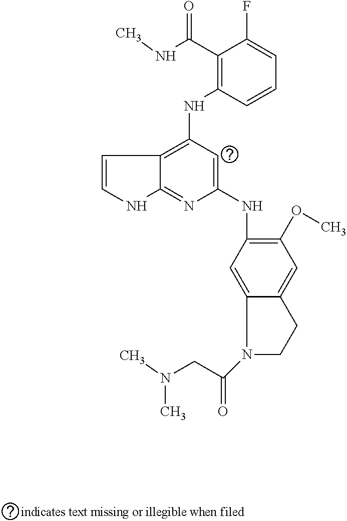 [SMILES: CNC(=O)c1c(F)cccc1Nc1cc(Nc2cc3c(cc2OC)CCN3C(=O)CN(C)C)nc2[nH]ccc12]

GSK1838705A and other related IGF-1R inhibitor small molecules and their methods of preparation can be found in U.S. Pat. No. 7,981,903, which is hereby incorporated by reference in its entirety. GSK1838705A and the other IGF-1R inhibitors described therein are predicted to have activity in the activity measures or assessments for the treatment of TED described herein.

### Example 18

BMS-554417

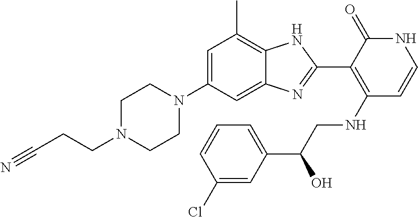 [SMILES: Cc1cc(N2CCN(CCC#N)CC2)cc2nc(-c3c(NC[C@@H](O)c4cccc(Cl)c4)cc[nH]c3=O)[nH]c12]

BMS-554417 and other related IGF-1R inhibitor small molecules and their methods of preparation can be found in U.S. Pat. No. 7,081,454, which is hereby incorporated by reference in its entirety. BMS-554417 and the other IGF-1R inhibitors described therein are predicted to have activity in the activity measures or assessments for the treatment of TED described herein.

### Example 19

NVP-ADW742

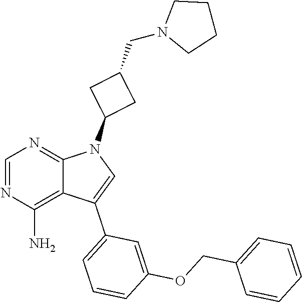 [SMILES: Nc1ncnc2c1c(-c1cccc(OCc3ccccc3)c1)cn2[C@H]1C[C@H](CN2CCCC2)C1]

NVP-ADW742 and other related IGF-1R inhibitor small molecules and their methods of preparation can be found in U.S. Pat. No. 7,326,699, which is hereby incorporated by reference in its entirety. NVP-ADW742 and the other IGF-1R inhibitors described therein are predicted to have activity in the activity measures or assessments for the treatment of TED described herein.

### Example 20

GSK1904529A

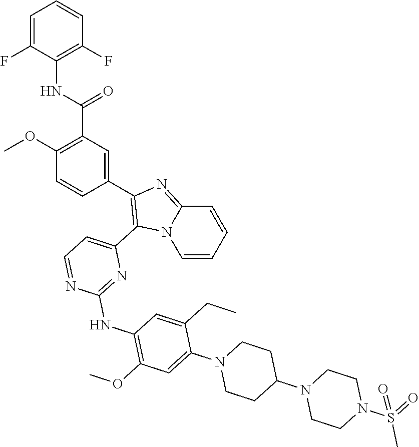 [SMILES: CCc1cc(Nc2nccc(-c3c(-c4ccc(OC)c(C(=O)Nc5c(F)cccc5F)c4)nc4ccccn34)n2)c(OC)cc1N1CCC(N2CCN(S(C)(=O)=O)CC2)CC1]

GSK1904529A and other related IGF-1R inhibitor small molecules and their methods of preparation can be found in U.S. Pat. No. 8,093,239, which is hereby incorporated by reference in its entirety. GSK1904529A and the other IGF-1R inhibitors described therein are predicted to have activity in the activity measures or assessments for the treatment of TED described herein.

### Example 21

KW-2450

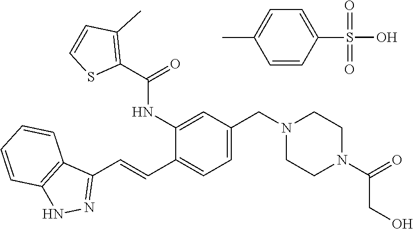 [SMILES: Cc1ccsc1C(=O)Nc1cc(CN2CCN(C(=O)CO)CC2)ccc1/C=C/c1n[nH]c2ccccc12 ; Cc1ccc(S(=O)(=O)O)cc1]

KW-2450, shown above as the tosylate salt but not limited thereto, and other related IGF-1R inhibitor small molecules and their methods of preparation can be found in WO2006080450, U.S. Pat. No. 7,605,272, and WO2011158931, which are hereby incorporated by reference in their entireties. KW-2450 and the other IGF-1R inhibitors described herein are predicted to have activity in the activity measures or assessments for the treatment of TED described herein.

### Example 22

PL-225B

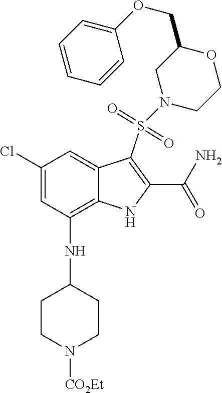 [SMILES: CCOC(=O)N1CCC(Nc2cc(Cl)cc3c(S(=O)(=O)N4CCO[C@H](COc5ccccc5)C4)c(C(N)=O)[nH]c23)CC1]

PL-225B and other related IGF-1R inhibitor small molecules and their methods of preparation can be found in WO2012145471 and WO2012007926, which is hereby incorporated by reference in its entirety. PL225B selectively inhibits IGF-1 R, resulting in inhibition of tumor cell proliferation and the induction of tumor cell apoptosis in IGF-1 R-overexpressing tumor cells. PL-225B and the other IGF-1R inhibitors described herein are predicted to have activity in the activity measures or assessments for the treatment of TED described herein.

### Example 23

I-18, nordihydroguaiaretic acid (NDGA)/Masoprocol, Actinex

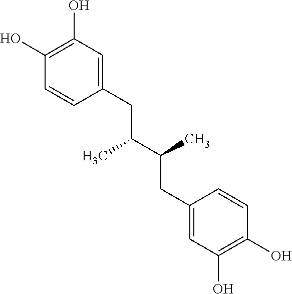 [SMILES: C[C@H](Cc1ccc(O)c(O)c1)[C@@H](C)Cc1ccc(O)c(O)c1]

INSM-18, nordihydroguaiaretic acid (NDGA) (shown above with relative stereochemistry, in which case it is also referred to as Masoprocol or Actinex, but not limited thereto) referred to in this Example as INSM-18, and other related IGF-1R inhibitor small molecules and their methods of preparation can be found at least in U.S. Pat. No. 2,373,192, which is hereby incorporated by reference in its entirety. INSM-18 directly inhibits activation of IGF-1 R and the c-erbB2/HER2/neu receptor, resulting in decreased proliferation of susceptible tumor cell populations. INSM-18 and the other IGF-1R inhibitors described herein are predicted to have activity in the activity measures or assessments for the treatment of TED described herein.

### Example 24

AZD3463

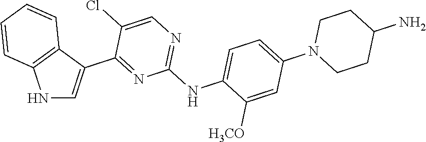 [SMILES: COc1cc(N2CCC(N)CC2)ccc1Nc1ncc(Cl)c(-c2c[nH]c3ccccc23)n1]

AZD3463 and other related IGF-1R inhibitor small molecules and their methods of preparation can be found in U.S. Pat. No. 8,461,170, which is hereby incorporated by reference in its entirety. AZD3463 is a potent ALK/IGF-1 R inhibitor, resulting in inhibition of neuroblastoma growth by overcoming crizotinib resistance and inducing apoptosis. AZD3463 and the other IGF-1R inhibitors described herein are predicted to have activity in the activity measures or assessments for the treatment of TED described herein.

### Example 25

AZD9362

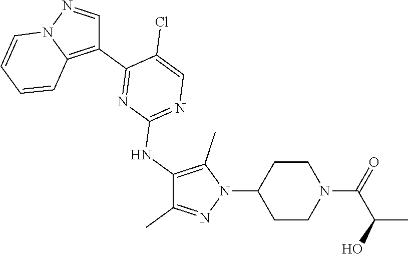 [SMILES: Cc1nn(C2CCN(C(=O)[C@@H](C)O)CC2)c(C)c1Nc1ncc(Cl)c(-c2cnn3ccccc23)n1]

AZD9362 and other related IGF-1R inhibitor small molecules and their methods of preparation can be found in Degorce, S L et al., “Discovery of a Potent, Selective, Orally Bioavailable, and Efficacious Novel 2-(Pyrazol-4-ylamino)-pyrimidine Inhibitor of the Insulin-like Growth Factor-1 Receptor (IGF-1R),” J Med Chem (2016), 59(10), 4859-4866., which is hereby incorporated by reference in its entirety. AZD9362 is a dual inhibitor of IGF-1R/InsR. AZD9362 and the other IGF-1R inhibitors described herein are predicted to have activity in the activity measures or assessments for the treatment of TED described herein.

### Example 26

B1885578

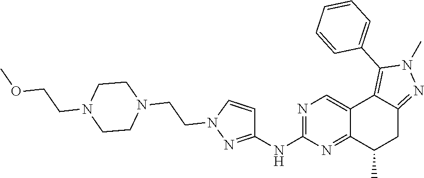

BI885578 and other related IGF-1R inhibitor small molecules and their methods of preparation can be found in U.S. Ser. No. 10/414,769, U.S. Pat. No. 9,150,578, and Sanderson M P et al., “BI 885578, a Novel IGF1R/INSR Tyrosine Kinase Inhibitor with Pharmacokinetic Properties That Dissociate Antitumor Efficacy and Perturbation of Glucose Homeostasis,” Mol Cancer Ther 2015 Dec.; 14(12):2762-72, which are hereby incorporated by reference in its entirety. BI885578 is an IGF1R/INSR tyrosine kinase inhibitor distinguished by rapid intestinal absorption and a short in vivo half-life as a result of rapid metabolic clearance, resulting in inhibition of cell proliferation and induction of apoptosis in tumors. BI885578 and the other IGF-1R inhibitors described herein are predicted to have activity in the activity measures or assessments for the treatment of TED described herein.

### Example 27

B1893923

BI893923 and other related IGF-1R inhibitor small molecules and their methods of preparation can be found in U.S. Pat. No. 8,546,443 and Titze IOI et al., “An allometric pharmacokinetic/pharmacodynamics model for BI893923, a novel IGF-1 receptor inhibitor,” Cancer Chemother Pharmacol 2017 Mar.; 79(3):545-558, which is hereby incorporated by reference in its entirety. BI893923 is an IGF1R/INSR tyrosine kinase inhibitor demonstrating anti-tumor efficacy and good tolerability. BI893923 and the other IGF-1R inhibitors described herein are predicted to have activity in the activity measures or assessments for the treatment of TED described herein.

### Example 28

XL-228

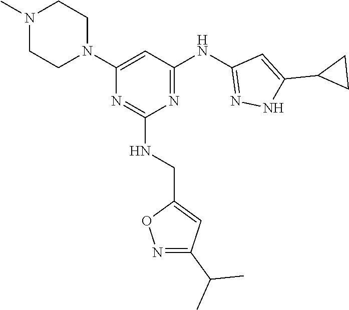 [SMILES: CC(C)c1cc(CNc2nc(Nc3cc(C4CC4)[nH]n3)cc(N3CCN(C)CC3)n2)on1]

XL-228 and other related IGF-1R inhibitor small molecules and their methods of preparation can be found in US20090232828, which is hereby incorporated by reference in its entirety. XL-228 is a broad protein kinase inhibitor that contributes to cell proliferation, cell survival, and resistance to cytotoxic agents. XL-228 and the other IGF-1R inhibitors described herein are predicted to have activity in the activity measures or assessments for the treatment of TED described herein.

### Example 29

A-928605

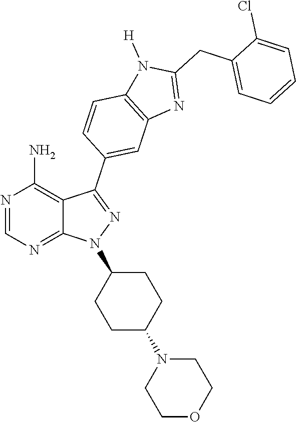 [SMILES: Nc1ncnc2c1c(-c1ccc3[nH]c(Cc4ccccc4Cl)nc3c1)nn2[C@H]1CC[C@H](N2CCOCC2)CC1]

A-928605 and other related IGF-1R inhibitor small molecules and their methods of preparation can be found in U.S. Pat. No. 7,772,231 and WO2007079164, which are hereby incorporated by reference in its entirety. A-928605 is a potent inhibitor of IGF-IR both on the purified enzyme and intracellular IGF-IR phosphorylation. A-928605 and the other IGF-1R inhibitors described herein are predicted to have activity in the activity measures or assessments for the treatment of TED described herein.

### Example 30

Istiratumab (MM-141)

Istiratumab and other related IGF-1R inhibitor antibodies and their methods of preparation can be found in U.S. Pat. No. 8,476,409, which is hereby incorporated by reference in its entirety.

Heavy Chain CDRs—Istiratumab

| HCDR1 | HCDR2 | HCDR3 |
|---|---|---|
| GFMFSR | ISGSGGAT | DFYQILT |
| YPMH | PYADSVKG | GNAFDY |
| (SEQ ID | (SEQ ID | (SEQ ID |
| NO: 67) | NO: 68) | NO: 69) |

Light Chain CDRs—Istiratumab

| LCDR1 | LCDR2 | LCDR3 |
|---|---|---|
| RASQGISSYLA | AKSTLQS | QQYWTFPLT |
| (SEQ ID | (SEQ ID | (SEQ ID |
| NO: 70) | NO: 71) | NO: 72) |

| | |
|---|---|
| Heavy Chain (HC) | EVQLLQSGGGLVQPGGSLRL |
| | SCAASGFMFSRYPMHWVRQA |
| | PGKGLEWVGSISGSGGATPY |
| | ADSVKGRFTISRDNSKNTLY |
| | LQMNSLRAEDTAVYYCAKDF |
| | YQILTGNAFDYWGQGTTVTV |
| | SSASTKGPSVFPLAPSSKST |
| | SGGTAALGCLVKDYFPEPVT |
| | VSWNSGALTSGVHTFPAVLQ |
| | SSGLYSLSSVVTVPSSSLGT |
| | QTYICNVNHKPSNTKVDKKV |
| | EPKSCDKTHTCPPCPAPELL |
| | GGPSVFLFPPKPKDTLMISR |
| | TPEVTCVVVDVSHEDPEVKF |
| | NWYVDGVEVHNAKTKPREEQ |
| | YNSTYRVVSVLTVLHQDWLN |
| | GKEYKCKVSNKALPAPIEKT |
| | ISKAKGQPREPQVYTLPPSR |
| | EEMTKNQVSLTCLVKGFYPS |
| | DIAVEWESNGQPENNYKTTP |
| | PVLDSDGSFFLYSKLTVDKS |
| | RWQQGNVFSCSVMHEALHNH |
| | YTQKSLSLSPGGGGGSGGGG |
| | SGGGGSQVQLVQSGGGLVQP |
| | GGSLRLSCAASGFTFDDYAM |
| | HWVRQAPGKGLEWVAGISWD |
| | SGSTGYADSVKGRFTISRDN |
| | AKNSLYLQMNSLRAEDTALY |
| | YCARDLGAYQWVEGFDYWGQ |
| | GTLVTVSSASTGGGGSGGGG |
| | SGGGGSGGGGSSYELTQDPA |
| | VSVALGQTVRITCQGDSLRS |
| | YYASWYQQKPGQAPVLVIYG |
| | KNNRPSGIPDRFSGSTSGNS |
| | ASLTITGAQAEDEADYYCNS |
| | RDSPGNQWVFGGGTKVTVLG |
| | (SEQ ID NO: 73) |
| Light Chain (LC) | DIQMTQSPSSLSASLGDRVT |
| | ITCRASQGISSYLAWYQQKP |
| | GKAPKLLIYAKSTLQSGVPS |
| | RFSGSGSGTDFTLTISSLQP |
| | EDSATYYCQQYWTFPLTFGG |
| | GTKVEIKRTVAAPSVFIFPP |
| | SDEQLKSGTASVVCLLNNFY |
| | PREAKVQWKVDNALQSGNSQ |
| | ESVTEQDSKDSTYSLSSTLT |
| | LSKADYEKHKVYACEVTHQG |
| | LSSPVTKSFNRGEC |
| | (SEQ ID NO: 74) |

Some embodiments of the disclosure are anti-IGF-1R inhibitor mAbs or antigen binding fragments thereof, comprising a heavy chain comprising a variable heavy chain CDR1, a variable heavy chain CDR2, and a variable heavy chain CDR3, wherein the variable heavy chain CDR1 comprises an amino acid sequence SEQ ID NO:67, the variable heavy chain CDR2 comprises an amino acid sequence SEQ ID NO:68; and the variable heavy chain CDR3 comprises an amino acid sequence SEQ ID NO:69 or at least a CDR with at least 80% of sequence identity after optimal alignment with SEQ ID NO:67, SEQ ID NO:68, and SEQ ID NO:69.

The anti-IGF-1R inhibitor mAbs or antibody or antigen binding fragment thereof may additionally comprise a light chain which is paired with the heavy chain to form an antigen binding domain. In some embodiments, the light chain comprises a variable light chain CDR1, a variable light chain CDR2, and a variable light chain CDR3, wherein the variable light chain CDR1 comprises an amino acid sequence SEQ ID NO:70, the variable light chain CDR2 comprises an amino acid sequence SEQ ID NO:71; and the variable light chain CDR3 comprises an amino acid sequence SEQ ID NO:72 or at least a CDR with at least 80% of homology after optimal alignment with SEQ ID NO:70, SEQ ID NO:71, and SEQ ID NO:72.

In some embodiments, the anti-IGF-1R inhibitor mAbs or antigen binding fragment thereof comprises a heavy chain amino acid sequence of SEQ ID NO:73 or at least a heavy chain with at least 85%, 90%, 95%, 97%, 98%, or 99% of sequence identity after optimal alignment with SEQ ID NO:73. Alternatively, or in addition, the anti-IGF-1R inhibitor mAbs or antigen binding fragment thereof may comprise a light chain having an amino acid sequence of SEQ ID NO:74 or at least a heavy chain with at least 85%, 90%, 95%, 97%, 98%, or 99% of sequence identity after optimal alignment with SEQ ID NO:74.

Measures of Idiopathic Orbital Inflammation (101)

### Example 31

Proposed Phase 1 clinical trial of IGF-1R inhibitors for the treatment of idiopathic orbital inflammation.

Idiopathic orbital inflammation (IOI), also known as orbital pseudotumor, orbital inflammatory pseudotumors (OIP), orbital inflammatory syndrome (OIS), idiopathic orbital inflammatory pseudotumor (IOIP), or nonspecific orbital inflammation (NSOI) is a benign, space-occupying, and non-infectious inflammatory condition of the orbit but may extend in the pen-orbital area. There is no identifiable infectious, systemic, or neoplastic disorder. It is the third most common orbital disease in adults, after thyroid orbitopathy and lymphoproliferative diseases. A significant number of orbital inflammations may be associated with systemic conditions or remote organ dysfunction. Categories of orbital pseudotumor according to location include anterior, diffuse, posterior, or apical. Other classifications include myositis, dacryoadenitis, periscleritis, perineuritis, and focal mass.

The most common ophthalmic findings are periorbital edema and blepharoptosis. A palpable mass may be present. On orbital radiography, common findings are dacryoadenitis, orbital mass, or myositis. Headache, emesis, anorexia, lethargy, and fever are the most common systemic signs. Additionally, there are likely associations with iritis, uveitis, disc edema, and peripheral eosinophilia.

See, e.g., Ronquillo Y and Patel B C, “Nonspecific Orbital Inflammation (NSOI),” StatPearls, updated Feb. 19, 2020; accessed May 28, 2020.

Insulin-like Growth Factor-1 Receptor (IGF-1R):

The insulin-like growth factor-1 receptor (IGF-1R) is a tyrosine kinase cell surface receptor that shares approximately 60% overall homology with the insulin receptor (IR) (Schumacher, 1991). When activated by its ligands, insulin-like growth factor (IGF)-1 and IGF-2, IGF-1R regulates important cellular activities involving cell proliferation, differentiation, and inflammation (Khandwala, 2000, Li, 2018, Ullrich, 1986).

IGF-1R Inhibitors, e.g., Teprotumumab:

In vitro, IGF-1R inhibitors (antagonists) have shown the ability to block signaling through multiple signal transduction pathways (Akt, MAPK, ERK, etc.), decrease the expression of cytokines and reduce secretion of disease related GAGs (Pritchard, 2003; Tsui, 2008; Smith and Hoa, 2004; Chen, 2014). TED and IOI have common disease features. By blocking signaling and down-regulating IGF-1R in fibroblasts, myofibroblasts, fibrocytes and cells of the immune system, teprotumumab has the potential to specifically resolve the key underlying pathophysiology of IOI, thereby reducing the severity and progression of the disease. The following clinical protocol uses teprotumumab, a fully human monoclonal antibody (mAb) IGF-1Ri developed for the treatment of thyroid eye disease (TED), as an example, but other IGF-1Ris may be used; examples of such inhibitors, and suitable dose ranges and modes of administration are set forth herein, and yet other IGF-1Ris are known in the art.

Objectives/Endpoints:

Objectives/endpoints of this study may include:
to investigate the safety, tolerability, and the effect on IGF-1, inflammatory and fibrotic biomarkers of IGF-1R inhibitors (IGF-1Ri), for example any of those disclosed herein, for example teprotumumab administered once every 3 weeks (q3W) for 15 to 24 weeks in the treatment of subjects with IOI.
to evaluate the safety of IGF-1Ri, e.g., teprotumumab, versus placebo on the proportion of subjects who experience a Treatment Emergent Adverse Event (TEAE) through e.g. Week 36 in subjects with IOI.
to evaluate recurrence number [e.g., up to 12 months]: frequency of occurrence of clinical condition showing signs of inflammation recurrence, during or after cessation of therapy.
to evaluate recurrence time [e.g., up to 12 months]: mean time of occurrence of clinical condition showing signs of inflammation recurrence, during or after cessation of therapy.
to evaluate the change from baseline over time in secreted proteins associated with the IGF-1 pathway, inflammation, and fibrosis in serum.
to evaluate change from baseline over time in patient-reported pain using a visual analog scale.
to evaluate change from baseline over time in periorbital edema/swelling.
to evaluate change from baseline over time in any one of the following measures:
   proptosis
   diplopia
   EOM restriction
   red eye
   chemosis
   ptosis
   periorbital edema
   blepharoptosis
   dacryoadenitis
   orbital mass
   myositis
   systemic signs such as headache, emesis, anorexia, lethargy, and/or fever.
   iritis, uveitis, disc edema, and/or peripheral eosinophilia.
to evaluate the change from baseline over time through Week 24 in IGF-1R protein expression on Peripheral Blood Mononuclear Cells (PBMC).
to evaluate the effect of IGF-1Ri, e.g., teprotumumab, on the mean change from baseline to Week 15 in erythrocyte sedimentation rate (ESR).
To evaluate the effect of IGF-1Ri, e.g., teprotumumab, on the mean change from baseline to Week 15 in high sensitivity C-Reactive Protein (hsCRP).

Optionally, the pharmacokinetics (PK) and immunogenicity of IGF-1Ri will also be evaluated. To assess safety and tolerability of IGF-1Ri including adverse events (AEs), adverse events of special interest (AESI) (including hyperglycemia, hearing impairment, infusion reaction and muscle spasms), concomitant medication use, vital signs, clinical safety laboratory evaluations, inflammatory laboratory evaluations, and immunogenicity may be evaluated.

Study Design:

In this embodiment, a randomized, double-blind, placebo controlled, repeat-dose, proof of mechanism, multicenter study is envisioned. Again, teprotumumab is disclosed as an exemplary test article, but other IGF-1Ris are disclosed herein and known in the art, and may be used. Furthermore, variations on the following protocol may be implemented by those skilled in the art.

Subjects will be screened for the study within 4 weeks prior to the Baseline (Day 1) Visit. Approximately e.g. 20 subjects who meet the study eligibility criteria will be randomized on Day 1 in a 3:1 ratio to receive 5-8 infusions of test article teprotumumab (10 mg/kg for the first infusion and 20 mg/kg for the remaining infusions) or placebo q3W. During the double-blind Treatment Period, study drug will be infused on Day 1 (Baseline), and Weeks 3, 6, 9, and 12 (and optionally more every 3 weeks) with a comprehensive visit at Week 15 or the final week. All study drug dosing will be performed at the clinic under the supervision of clinic staff, and at any scheduled infusion, the infusion rate may be reduced or the dose may be interrupted or held based on tolerability. On each dosing day, scheduled assessments (except for adverse event [AE] and concomitant medication use monitoring, which will be monitored throughout the clinic visit) will be completed prior to study drug infusions. After each of the first 2 infusions, subjects will be contacted by phone/email the following day. Additional phone/email contacts and clinic visits may also be conducted for any subject experiencing an infusion-associated event.

At the end of the Treatment Period (e.g. Week 15 or week 24), subjects will enter a Follow-Up Period, during which study drug will not be administered, and one or more clinic visits will be scheduled for subsequent weeks.

Subjects who prematurely discontinue study drug dosing prior to the final week of the Treatment Period will return to the clinic and undergo the scheduled assessments and will be encouraged to remain in the study and participate in the Follow-Up Period. An overview of the study design is presented in the schematic below.

### Example of a 15—Week Treatment Period Protocol

| | | Double-Blind Treatment Period | | | | | | Follow-Up | | |
|---|---|---|---|---|---|---|---|---|---|---|
| | | Test Article or Placebo^{1, 2} | | | | | | Period | | |
| Screen | Baseline | 15 Weeks | | | | | | 21 Weeks^{3, 4} | | |
| | * | * | * | * | * | | | | | |
| 4 weeks | Day 1 ⁵ | W 3 ⁵ | W 6 | W 9 | W 12 | W 15⁶ | W 18³ | W 24⁴ | W 30^{3, 4} | W 36^{4, 7} |
| predose | | | | | M 3 | | | M 6 | | M 9 |

| | |
|---|---|
| Randomization¹ | -Study Week/Month- |

* Infusion of study drug.
¹Subjects may be randomized in, e.g., a 1:1 or 3:1 ratio
a. Teprotumumab 20 mg/kg (10 mg/kg on Day 1 followed by 20 mg/kg q3W for the remaining 4 infusions); or
b. Placebo (placebo q3W for all 5 infusions).
²Visit windows are ±3 days for Weeks 3, 6, 9, 12, 15, and 18.
³All subjects will be contacted via phone or email at Week 18 and 30 to inquire how they subject is doing and to ask WOCBP about their menstrual cycle.
⁴Visit windows of ±7 days at Weeks 24, 30, and 36.
⁵ All subjects will be contacted by phone/email the day following the first (Day 1) and second (Week 3) infusions for safety and tolerability assessments; additional phone/email contacts will occur the day after any clinic visit where a subject experiences an infusion-related adverse event.
⁶If a subject prematurely discontinues prior to completing the Treatment Period, they will return to the clinic visit and undergo the Week 15 assessments. Subjects will be encouraged to continue in the 21-week follow-up.
⁷If a subject prematurely discontinues prior to completing the follow-up period, they will return to the clinic visit and undergo the Week 36 assessments.

Subject Population:

Male and non-pregnant female subjects between the ages of 18 and 80 years, inclusive, with IOI will be enrolled.

Inclusion Criteria:

Eligible subjects must meet/provide all of the following criteria:
Written informed consent.
Male or female subject between the ages of 15 and 80 years, inclusive, at Screening. Diagnosis of IOI;
Diabetic subjects must have HbA1c≤8.0%, with no new diabetic medication [oral or insulin] or more than a 10% change in the dose of a currently prescribed diabetic medication within 60 days prior to Screening.
Women of childbearing potential (including those with an onset of menopause <2 years prior to Screening, non-therapy-induced amenorrhea for <12 months prior to Screening, or not surgically sterile [absence of ovaries and/or uterus]) must have a negative serum pregnancy test at Screening and negative urine pregnancy tests at all protocol-specified timepoints (i.e., prior to each dose and throughout subject's participation in Follow Up Period); subjects who are sexually active with a non-vasectomized male partner must agree to use 2 reliable forms of contraception during the trial, one of which is recommended to be hormonal, such as an oral contraceptive. Hormonal contraception must be started at least one full cycle prior to Baseline and continue for 180 days after the last dose of study drug. Highly effective contraceptive methods (with a failure rate less than 1% per year), when used consistently and correctly, includes implants, injectables, combined oral contraceptives, some intrauterine devices (IUDs), sexual abstinence or vasectomized partner.
Male subjects must be surgically sterile or, if sexually active with a female partner of childbearing potential, must agree to use a barrier contraceptive method from Screening through 180 days after the last dose of study drug.
Subject is willing and able to comply with the prescribed treatment protocol and evaluations for the duration of the study.

Exclusion Criteria:

Subjects may be ineligible for study participation if they meet any of the following criteria:
Age <15 years
Diagnosis of diabetes mellitus, collagen vascular diseases, vasculitides, or glaucoma;
Biopsy of lacrimal gland denoting a specific diagnosis (i.e. the term “idiopathic” does not apply)
Patients who have lost one eye
Bilateral disease
Abnormal thyroid-stimulating hormone
Corticosteroid use for conditions other than IOI within 4 weeks prior to Screening (topical steroids for dermatological conditions and inhaled steroids are allowed).
Previous treatment with rituximab (Rituxan® or MabThera®) within 12 months prior to the first infusion.
Use of any other non-steroid immunosuppressive agent, cytotoxic or anti-fibrotic drug within 4 weeks of screening other than anti-malarial. This includes cyclophosphamide, azathioprine (Imuran), methotrexate or other immunosuppressive or cytotoxic medication except mycophenolate mofetil or mycophenolic acid [Myfortic].
Use of an investigational agent for any condition within 180 days prior to Screening or anticipated use during the course of the trial.
Subjects with medical conditions associated with increased risk of complications from bleeding, including history of hematological diseases such as anemia, acquired platelet disorders, coagulation disorders, including drug-induced thrombocytopenia, idiopathic thrombocytopenia, or von Willebrand's Disease.
Malignant condition in the past 5 years (except successfully treated basal/squamous cell carcinoma of the skin or cervical cancer in situ).
Pregnant or lactating women.
Current drug or alcohol abuse, or history of either within the previous 2 years, in the opinion of the Investigator or as reported by the subject.
Biopsy-proven or clinically suspected inflammatory bowel disease (e.g., diarrhea with or without blood or rectal bleeding associated with abdominal pain or cramping/colic, urgency, tenesmus, or incontinence for more than 4 weeks without a confirmed alternative diagnosis OR endoscopic or radiologic evidence of enteritis/colitis without a confirmed alternative diagnosis).
Known hypersensitivity to any of the components of teprotumumab or prior hypersensitivity reactions to mAbs.
Previous enrollment in this study or participation in a prior teprotumumab clinical trial.
HIV, hepatitis C or hepatitis B infections.
Subjects with organ transplant (including allogeneic and autologous marrow transplant).
Alanine aminotransferase (ALT) or aspartate aminotransferase (AST)>2.5 times the upper limit of normal (ULN) or eGFR of <30 ml/min/1.73 m2 at screening.
Platelets <120 K/μL.
Hemoglobin less than 10 mg/dl.
Any other condition that, in the opinion of the Investigator, would preclude inclusion in the study.

Dose Regimen/Route of Administration:

All study drug dosing will be performed at the clinic under the supervision of clinic staff. On Day 1 of the double-blind Treatment Period, subjects will be randomized to in a 3:1 ratio to receive infusions of either: Teprotumumab 20 mg/kg (10 mg/kg on Day 1 followed by 20 mg/kg q3W for the remaining infusions), or Placebo (q3W for all infusions).

The infusion rate may be reduced and the dose may be interrupted or held based on tolerability. The first and second infusions will be administered over approximately 90 minutes (but not less than 80 minutes). Subsequent infusions will be administered over approximately 60 minutes (but not less than 50 minutes), providing there are no significant infusion-associated events.

Dosage Form and Strength Formulation:

Teprotumumab 500 mg will be provided in single-dose 20 mL glass vials as a freeze-dried powder. Each vial of teprotumumab will be reconstituted with 10 mL of sterile water for injection. The resulting solution will have an approximate concentration of 50 mg/mL teprotumumab. Reconstituted teprotumumab solution will be further diluted in 0.9% (w/v) sodium chloride (NaCl) solution prior to administration.

Doses up to 1800 mg will be administered in a total infusion volume of 100 mL, and those above 1800 mg will be administered in a total infusion volume of 250 mL. To maintain a constant volume in the infusion bags, a volume equal to the volume of teprotumumab to be placed into the infusion bag will be first removed from the infusion bag using a sterile syringe and needle. The appropriate volume of reconstituted drug product solution based on the subject's dose and body weight will be withdrawn and the teprotumumab reconstituted drug product solution will be diluted with normal saline (0.9% NaCl) in the infusion bag.

Placebo will consist of a normal saline (0.9% NaCl) solution and will be administered in 100 mL or 250 mL infusion bags, as appropriate, per weight-based dosing volumes.

Duration of Treatment:

The planned duration of the Treatment Period may be 15 weeks (3 months) to 24 weeks. At Week 15 or Week 24, all subjects will enter a Follow-Up Period of, e.g. 21-weeks.

Criteria for Evaluation:

At the Baseline (Day 1) Visit, the IOI will be assessed. The steps to the process include assigning a probability of improvement for the subject and ranges from 0 (no improvement) to 1 (marked improvement).

Blood samples for teprotumumab PK assessment will be collected prior to, and at the end of, the infusion on Day 1 and Weeks 3 and 12, and a single sample at Week 15 during the Treatment Period; in addition, a single sample will be collected at Week 24. Additional samples may be taken at later time points.

Blood samples will be collected prior to the infusion to analyze for biomarkers of the IGF-1 pathway, inflammation and fibrosis e.g., on Day 1 and Weeks 3, 15 and 24. Additional samples may be taken at later time points.

Blood samples will be collected prior to the infusion to analyze for Anti-Drug Antibodies (ADA) at Baseline, Weeks 3, 12, 15, and 24. Additional samples may be taken at later time points.

Safety will be assessed via AE and concomitant medication use monitoring, immunogenicity testing, physical examinations, vital signs, clinical safety laboratory evaluations (complete blood count and chemistry (including HbA1c), clinical inflammatory laboratory evaluations (hsCRP and ESR), pregnancy testing (if applicable), and a screening electrocardiograms (ECG).

Specific efficacy measures (e.g., proptosis, diplopia, extraocular muscle (EOM) restriction, red eye, chemosis, ptosis, periorbital edema, blepharoptosis, dacryoadenitis, orbital mass, myositis, and systemic signs such as headache, emesis, anorexia, lethargy, and/or fever) will be assessed using scales and criteria known in the art. Imaging will be performed to detect involvement of lacrimal glad, superior rectus muscle, lateral rectus muscle, or the inferomedial orbit. Histologic analysis will be performed to detect whether the type of inflammation is nonspecific nongranulomatous, chronic, sclerosing or nonspecific granulomatous.

Statistical Analyses:

The following endpoints, and others, may be statistically analyzed:
1. The proportion of subjects who experience a TEAE (as defined by Common Terminology Criteria for Adverse Events (CTCAE) 5.0) through Week 36 in subjects with IOI.
2. Mean change from Baseline to Week 12 in any of the relevant measures.
3. The change from baseline in biomarkers of the IGF-1 pathway, inflammation, and fibrosis.
4. The change from baseline in transcriptomics associated with IGF-1R pathway, inflammation, and fibrosis.
5. The change from baseline in IGF-1R protein expression in skin biopsies and PBMCs.
6. Pharmacokinetics of test article and, optionally, metabolites.
7. The incidence of ADA and titer levels (if test article is an antibody or fragment).
8. Incidence of AEs, and AESI (e.g., hyperglycemia, hearing impairment, infusion reaction and muscle spasms).
9. Concomitant medication use.
10. Vital signs.
11. Clinical safety laboratory evaluations.

Statistical Analysis on Efficacy and Safety Parameters:

The analysis on (1) may be conducted using safety analysis set, consisting of all subjects who receive at least one dose of study drug (full or partial dose). The number and percentage of subjects who experience a Grade 3 or higher AE may be summarized by treatment group.

Efficacy analyses may be conducted using ITT analysis set, consisting of all subjects randomized to treatment, with select endpoints also summarized by more restrictive analysis sets, including subjects who received a minimum amount of treatment and have available data. Descriptive summaries for observed and change from baseline for efficacy measures may be summarized by treatment group and visit.

Analysis of efficacy data will calculate the probability of a subject improving, using appropriate statistical methods, for example the formula in FIG. 3 of Khanna et al (2016). Additionally, probability as a continuous measure may also be summarized with mean at each scheduled assessment.

Descriptive summaries for observed and change from baseline in IGF-1R pathway, inflammatory and fibrotic biomarkers may be summarized by treatment group at each scheduled visit.

Descriptive summaries for observed and change from baseline in transcriptomic associated with IGF-1R inhibition may be summarized by treatment group at each scheduled visit.

Safety analyses may be performed using the safety analysis set.

The number and percentage of patients in each treatment group reporting at least one occurrence of a Treatment Emergent AE (TEAE), a TEAE of grade 3 or higher, a serious adverse event (SAE), a TEAE related to study drug, an AESI and a TEAE resulting in discontinuation of treatment may be summarized by treatment group. TEAEs may additionally be summarized by system organ class (SOC) and preferred term (PT).

Concomitant medications may be summarized by Anatomical Therapeutic Chemical (ATC) Level 4 term and preferred term (PT) using counts and percentage of subjects for each treatment group.

Descriptive summaries of observed and change from Baseline values may be presented for each vital sign parameter by treatment group and visit. A shift table for vital signs by Common Terminology Criteria for Adverse Events (CTCAE) grade and visit may be summarized by treatment group.

Safety laboratory assessments (hematology and chemistry (including HbA1c)), and change from baseline may be summarized by visit and treatment group using descriptive statistics. The laboratory assessment may be categorized as low, normal, and high based on their normal ranges. Shift tables using categories of low, normal, and high from baseline to each visit may be summarized by treatment group. Additionally, a shift table for glucose by CTCAE grade and visit may be summarized by treatment group. Summaries may be provided separately for hyperglycemia.

Alternatives:

The clinical trial described in Example 31 is provided to allow those skilled in the art to test the efficacy of any IGF-1R inhibitor. This clinical trial is described using a specific IGF-1R inhibitor, viz. Teprotumumab, a humanized monoclonal antibody. Those skilled in the art can understand that the scope of this trial extends to the testing of any IGF-1R inhibitor which may be antibodies, antibody fragments, or small molecules. Dosing of other IGF-1R inhibitors can be modified based on the type of inhibitor being used. Other IGF-1R inhibitors are expected to have qualitatively similar efficacy in treating or reducing the symptoms associated with IOI. Such modifications of this clinical trial are also intended to fall within the scope of the appended claims.

Results:

IGF-1R inhibitors, e.g., Teprotumumab, are expected to have efficacy in the treatment of IOI and related conditions. IGF-1R inhibitors are expected to have efficacy in IOI and its various symptoms and measures, including: frequency of recurrence, time to recurrence, proptosis, diplopia, extraocular muscle (EOM) restriction, red eye, chemosis, ptosis, periorbital edema, blepharoptosis, dacryoadenitis, orbital mass, myositis, and systemic signs such as headache, emesis, anorexia, lethargy, and/or fever. Treatment with IGF-1R inhibitor is expected to result in an altered transcriptome associated with the IGF-1 pathway, e.g., more closely resembling or associated with a non-diseases phenotype; altered secreted proteins associated with the IGF-1 pathway, e.g., more closely resembling or associated with a non-diseases phenotype; reduced inflammation and/or fibrosis in serum; altered IGF-1R protein expression on Peripheral Blood Mononuclear Cells (PBMC); reduced erythrocyte sedimentation rate (ESR); and reduced high sensitivity C-Reactive Protein (hsCRP).

### Example 32

### Animal Model to Test Efficacy of Treatment of Idiopathic Orbital Inflammation

Alternatively or in addition, a murine model may be used to study orbital inflammation; see Amarnani et al., “Characterization of a Murine Model of Oxazolone-Induced Orbital Inflammation,” Transl Vis Sci Technol. 2020 Jul. 16; 9(8):26. Inflammation is induced by injecting oxazolone into the mouse orbit. Resulting pathology has radiographic congruity between the murine model and human disease. After 72 hours, sensitized mice exhibit periorbital dermatitis and inflammation in the eyelids of the injected side. By one week, increased proptosis in the injected eye with significant eyelid edema is seen. By four weeks, inflammation and proptosis are decreased. At all three time points, mice demonstrate exophthalmos and periorbital edema. Histopathologically, populations of inflammatory cells including T cells, macrophages, and neutrophils share similarities with human disease. Proteomic changes in the levels of inflammatory and angiogenic markers correlate to the expected angiogenic, inflammatory, and fibrotic responses observed in humans. The murine model recapitulates some of the clinical features of idiopathic orbital inflammation, typified by inflammatory cell infiltration, orbital tissue expansion and remodeling, and subsequent fibrosis. Accordingly, this mouse model may serve as a viable preclinical model of acute orbital inflammation and diseases, such as IOI, which involve orbital inflammation, and to investigate potential treatments.

Objectives and Endpoints of Study Include:

Investigate effectiveness of various IGF1R inhibitors for treatment of IOI in a mouse model based on radiographical, histopathological, immunofluorescence, and cytokine array data.

Experimental Design:

Eight-week-old female BALB/c mice (Jackson Laboratory, Bar Harbor, Me., USA) is sensitized by a topical application of 2% oxazolone. Five days after sensitization, the right orbit is challenged with 5 microliters of 2% oxazolone solution. Uninjected left eyes serve as a control group for this study. Mice receiving oxazolone injections are expected to develop orbital inflammation at 72 hours and one week time points. Orbital inflammation is documented with observable clinical symptoms as well as magnetic resonance imaging (MRI), histopathology, immunofluorescence, and cytokine levels as described in Amarnani et al. Between 72 hour and one week time point, a subset of mice from each group may be euthanized for providing baseline data using the methods above. Mice are then treated with one or more appropriate doses of IGF1R inhibitor, for example as disclosed herein. After treatment, test and control mice are euthanized at three time points: 72 hours, one week, and four weeks, and a subset (e.g., 1 mouse) at each time point may undergo MRI just before euthanasia. MRI, histopathologic, immunofluorescence and cytokine array analysis may be performed. MRI, histopathology, immunofluorescence, and cytokine array analysis may provide a measure for effective treatment of orbital inflammation.

It is expected that treatment with an IGF1R inhibitor will decrease oxazolone-induced orbit inflammation in the mouse model, e.g., as shown by MRI, histopathology, immunofluorescence, and cytokine array analysis. This mouse model is a useful tool in preclinical testing of various IGF1R inhibitors for their safety and efficacy in reducing orbital inflammation.

### Example 33

### Perturbation of IGF-1R Signalling Pathways in IOI and TED

Messenger RNA (mRNA) signatures (gene expression profiling) had been previously shown to differentiate TED from other orbital inflammatory diseases, including idiopathic orbital inflammation. However, more recent studies have shown that four orbital inflammatory disease groups, including TED and IOI, demonstrated perturbation of the IGF-1R, PPARγ, adipocytokine and AMPK signalling pathways. This reinforces the notion that targeting IGF-1R is a viable therapeutic strategy in IOI. See, Verma R et al., “Enrichment of IGF-1R and PPARγ signalling pathways in orbital inflammatory diseases: steps toward understanding pathogenesis,” Br J Ophthalmol 0:1-6 (2021).

### OTHER EMBODIMENTS

The detailed description set-forth above is provided to aid those skilled in the art in practicing the present disclosure. However, the disclosure described and claimed herein is not to be limited in scope by the specific embodiments herein disclosed because these embodiments are intended as illustration of several aspects of the disclosure. Any equivalent embodiments are intended to be within the scope of this disclosure. Indeed, various modifications of the disclosure in addition to those shown and described herein will become apparent to those skilled in the art from the foregoing description, which do not depart from the spirit or scope of the present inventive discovery. Such modifications are also intended to fall within the scope of the appended claims.

What is claimed is:

1. A method of treating or reducing one or more effects of idiopathic orbital inflammation (IOI), or one or more symptoms thereof, in a subject with IOI, comprising administering to the subject an effective amount of an insulin-like growth factor 1 receptor (IGF-1R) inhibitor.

2. The method of claim 1, wherein the IOI is classified histopathologically as one of classic, granulomatous, sclerosing, and not otherwise specified (NOS), and/or is classified based on localization as one or more of diffuse, extraocular muscle, lacrimal gland, optic nerve, sclera and not otherwise specified (NOS).

3. The method of claim 1, wherein the one or more effects of IOI that is/are reduced is/are chosen from: eyelid edema, ptosis, proptosis, globe displacement, impairment of eye motility, disease progression, nongranulomatous chronic inflammation, sclerosing inflammation, and nonspecific granulomatous inflammation.

4. The method of claim 1, wherein the IGF-1R inhibitor is an antibody, or an antigen binding fragment thereof, or a small molecule or a salt or polymorph thereof.

5. The method of claim 4, wherein said IGF-1R inhibitor is chosen from teprotumumab, ganitumab, figitumumab, MEDI-573, cixutumumab, dalotuzumab, robatumumab, AVE1642, BII13022, xentuzumab, istiratumab, linsitinib, picropodophyllin, BMS-754807, BMS-536924, BMS-554417, GSK1838705A, GSK1904529A, NVP-AEW541, NVP-ADW742, GTx-134, AG1024, KW-2450, PL-2258, INSM-18, AZD3463, AZD9362, BI885578, BI893923, TT-100, XL-228, and A-928605.

6. The method of claim 4, wherein said IGF-1R inhibitor is an antibody, or an antigen binding fragment thereof.

7. The method of claim 6, wherein said IGF-1R inhibitor is a human, chimeric human, or humanized monoclonal antibody, or an antigen binding fragment thereof, suitable for human therapy.

8. The method of claim 6, wherein the antibody or antigen binding fragment thereof is administered intradermally, subcutaneously (SC), intravenously (IV), or by inhalation.

9. The method of claim 8, wherein the antibody or antigen binding fragment thereof is administered intravenously (IV) or subcutaneously (SC).

10. The method of claim 9, wherein the antibody or antigen binding fragment thereof is administered IV.

11. The method of claim 10, wherein the IGF-1R inhibitor is administered by IV infusion to the subject.

12. The method of claim 6, wherein said antibody is chosen from teprotumumab, ganitumab, figitumumab, MEDI-573, cixutumumab, dalotuzumab, robatumumab, AVE1642, BIIB022, xentuzumab, and istiratumab, or an antigen binding fragment of any of the foregoing.

13. The method of claim 1, wherein the IGF-1R inhibitor is an antibody, or antigen binding fragment thereof, that has a binding affinity (K_{D}) of 10⁻⁸ M or less for the IGF-1R.

14. The method of claim 1, wherein the antibody, or antigen binding fragment thereof, has an IC50 values for the IGF1 and IGF2 to IGF-1R of no more than 2 nM.

15. The method of claim 1, wherein said IGF-1R inhibitor is a small molecule, or a salt or polymorph thereof.

16. The method of claim 15, wherein said IGF-1R inhibitor, or salt or polymorph thereof, is dosed orally or intravenously.

17. The method of claim 15, wherein said IGF-1R inhibitor, or salt or polymorph thereof, is chosen from linsitinib, picropodophyllin, BMS-754807, BMS-536924, BMS-554417, GSK1838705A, GSK1904529A, NVP-AEW541, NVP-ADW742, GTx-134, AG1024, KW-2450, PL-2258, NVP-AEW541, NSM-18, AZD3463, AZD9362, BI885578, BI893923, TT-100, XL-228, and A-928605, or a salt or polymorph of any of the foregoing.

18. A pharmaceutical composition comprising an amount of an insulin-like growth factor 1 receptor (IGF-1R) inhibitor effective for treating or reducing one or more effects of idiopathic orbital inflammation (IOI), or one or more symptoms thereof, in a subject with IOI.

19. The pharmaceutical composition of claim 18, wherein said IGF-1R inhibitor is an antibody, or an antigen binding fragment thereof.

20. The pharmaceutical composition of claim 18, wherein said antibody is chosen from teprotumumab, ganitumab, figitumumab, MEDI-573, cixutumumab, dalotuzumab, robatumumab, AVE1642, BIIB022, xentuzumab, and istiratumab, or an antigen binding fragment of any of the foregoing.
